(19) United States
(12) Patent Application Publication
(10) Pub. No.: US 2023/0078007 A1
(43) Pub. Date: Mar. 16, 2023

(54) IN-SERIES SYNTHETIC RECEPTOR AND-GATE CIRCUITS FOR EXPRESSION OF A THERAPEUTIC PAYLOAD BY ENGINEERED CELLS

(71) Applicant: The Regents of the University of California, Oakland, CA (US)

(72) Inventors: Jasper Z. WILLIAMS, San Franisco, CA (US); Devan SHAH, San Franisco, CA (US); Wendell A. LIM, San Francisco, CA (US)

(21) Appl. No.: 17/800,872

(22) PCT Filed: Feb. 19, 2021
(86) PCT No.: PCT/US21/18780

§ 371 Date: Aug. 18, 2022

Related U.S. Application Data
(60) Provisional application No. 62/980,889, filed on Feb. 24, 2020.

(51) Int. Cl. A61K 35/17; C12N 5/0783; C12N 15/11; C12N 9/22; C12N 15/62; C12N 9/02; A61P 35/00; A61K 35/15; C07K 16/28; C07K 14/725; A61K 38/19; A61K 38/44; A61K 38/48; C07K 16/32

(52) U.S. Cl. CPC A61K 35/17; C12N 5/0636; C12N 15/11; C12N 9/22; C12N 15/625; C12N 9/0089; C12Y 115/01001; A61P 35/00; A61K 35/15; C07K 16/2818; C07K 16/2827; C07K 16/2803; C07K 14/7051; A61K 38/19; A61K 38/446; A61K 38/48; C07K 16/32; C12N 2310/20; C12N 2800/80; C12N 2840/00; C07K 2319/03; C07K 2317/622

(57) ABSTRACT

Provided herein is an in-series synthetic receptor circuit for dual-antigen AND-gate control over expression of a therapeutic payload by engineered cells. In some embodiments, the circuit may be composed of a first binding-triggered transcriptional switch, a second binding-triggered transcriptional switch and a therapeutic payload (e.g., a chimeric antigen receptor), where binding of the first binding-triggered transcriptional switch to a first antigen activates expression of the second binding-triggered transcriptional switch, and binding of the second binding-triggered transcriptional switch to a second antigen activates expression of the therapeutic payload. If the cell is an immune cell and the therapeutic payload is a chimeric antigen receptor, then the immune cell may be activated by binding of the chimeric antigen receptor to a third antigen. Methods of treatment using the cell also provided.

### CROSS-REFERENCING

This application claims the benefit of U.S. provisional application Ser. No. 62/980,889, filed on Feb. 24, 2020, which application is incorporated by reference in its entirety.

### GOVERNMENT RIGHTS

This invention was made with government support under grant no. U54 CA244438 awarded by The National Institutes of Health. The government has certain rights in the invention.

### INTRODUCTION

With the recent success of chimeric antigen receptor (CAR) T cell therapies, there has been an increased interest in using synthetic biology and cell engineering techniques to develop improved cellular therapies. Techniques used to engineer therapeutic cells have been based on introducing antigen-specific receptors into cells to impart them with antigen-dependent therapeutic responses. Specifically, T cells engineered to express CARs target and kill cancer cells that express their target antigen. However, the broader clinical application of therapeutic T cells to many cancer types is somewhat limited by the lack of targetable single antigens whose expression cleanly distinguishes cancer cells from healthy cells. This lack of antigen specificity is particularly true when targeting solid tumors—many of the potential CAR tumor antigen targets are also found in normal epithelial tissues, leading to observed toxic on-target off-tumor cross-reactions. Cancer cells differ only slightly from normal cells in their molecular features, thus it is not surprising that targeting a single antigen is often insufficient to discriminate against normal tissues. It is appreciated that combinatorial antigen pattern recognition could enable far more specific cancer (and other complex disease) cell targeting; however, there are limited options for engineering mammalian cells with these combinatorial sensing capabilities.

It has previously been shown that T cells can be engineered with a constitutively expressed synNotch receptor targeting a first antigen that controls expression of a CAR targeting a second antigen, and that these T cells function as robust AND-gates that only kill target cells that express both of the antigens (Roybal et al, Cell 2016 164: 770-779). Additionally, it has been shown that T cells can be engineered with a synNotch receptor targeting a cancer-associated antigen that controls expression of any genetically-encodable therapeutic payload and that these T cells are capable of delivering therapeutically-relevant levels of these biologic agents specifically to tumors to avoid side effects from systemic delivery (Roybal et al, Cell 2016 164: 770-779). However, both of these circuits, while they improve the precision of the therapy, may be insufficient for cancers that can only be targeted with any specificity using two or three antigens.

Thus, in some cases, it may be desirable to a deliver therapeutic payload with higher specificity. This disclosure addresses this issue.

### SUMMARY

Provided herein is an in-series synthetic receptor circuit for dual-antigen AND-gate control over expression of a therapeutic payload by engineered cells. In some embodiments, the circuit may be composed of a first binding-triggered transcriptional switch (BTTS), a second BTTS and a therapeutic payload (e.g., a chimeric antigen receptor), where binding of the first BTTS to a first antigen activates expression of the second BTTS, and binding of the second BTTS to a second antigen activates expression of the therapeutic payload. If the cell is an immune cell and the therapeutic payload is a chimeric antigen receptor, then the immune cell may be activated by binding of the chimeric antigen receptor to a third antigen. In these embodiments, the first, second and third antigens may be expressed on the surface of the same cell (e.g., a cancer cell) or in the same tumor and, as such, some embodiments of the circuit may require a minimum of three antigens for activation.

Methods of treatment are also provided.

### BRIEF DESCRIPTION OF THE DRAWINGS

FIG. 1A-C depict the two synNotch receptors that use orthogonal transcriptional domains to avoid cross-talk. In this example, it was first shown that T cells engineered with an anti-EGFR Gal4-VP64 synNotch In-Series with an anti-MET LexA-VP64 controlling mCherry expression only activated the fluorescent reporter in response to EGFR+/MET+ K562 or HCC827 target cells, but not cells expressing either single antigen.

FIG. 2A-E depict a circuit in which expression of an anti-Her2 CAR was put under control of a EGFR→MET In-Series synNotch circuit. In this example, it was found that T cells engineered with this circuit selectively were activated by and killed MET+/EGFR+/Her2+ K562 target cells, while sparing cells expressing Her2 alone or with only one of the two other antigens in the circuit.

FIG. 3A depicts the difference in results between the highly specific killing by a 3-input in-series circuit T cells and the indiscriminate killing of all Her2+ cells by constitutive anti-Her2 CAR T cells (FIG. 3).

FIG. 4A-B depict an in-Parallel 3 input AND gate, using the same antigen targets, that was also constructed. In this example, the EGFR and MET synNotch receptors were used to drive expression of separate components of a split a-Her2 CAR (the CAR requires a Her2 scFv-peptide fusion as an adapter).

FIG. 5A depicts results showing that the arrangement of the various genetic elements that make up the circuit on two separate plasmids can make a large difference in the ability to express the circuit in primary human T cells.

FIG. 6A-B depict circuits and results from circuits having different transcription factors. In this example, having the synNotch with the stronger transcription factor (Gal4-VP64) upstream of the synNotch with the weaker transcription factor (LexA-VP64) was important for inducing a strong mCherry induction in the presence of both synNotch antigens (FIG. 6).

FIG. 7A-G depict computationally enumerating combinatorial antigen pairs predicted to improve T cell discrimination of cancer vs normal cells. A. Single antigen targets for CAR T cells often show cross reactivity with subset of normal tissues. Combinatorial recognition circuits (AND, NOT, etc) could improve discrimination. B. Computational pipeline for identifying antigen pairs with improved tumor discrimination. For each cancer type (N=33), normalized RNAseq expression data is combined with RNAseq data for 34 normal tissues. All potential transmembrane antigen pairs are then evaluated for their potential to separate samples of a given tumor type from all normal samples in expression space. C. Scoring metric for discrimination based on separation distance and sample spread of tumor vs normal samples in 2D antigen space (see FIG. 14 and Methods). D. Distribution of tumor vs normal discrimination scores (F1) for top all possible single clinical antigens for each cancer type, and for top 10 antigen pairs (clinical:clinical, clinical:novel, or novel:novel) for each cancer type. F1 scores range between 0 (no sensitivity and specificity) and 1 (perfect precision and recall). E. Distribution of potential off-tumor toxicities (precision) for top all possible single clinical antigens for each cancer type, and for top 10 antigen pairs (clinical:clinical, clinical:novel, or novel:novel) for each cancer type. Precision values range between 0 (no specificity) and 1 (only cancer samples targeted). F. Improvement in tumor vs normal discrimination with dual antigen recognition by cancer type. F1 scores of best single clinical antigen vs best dual antigen pair from among the top 10 are plotted. This is an abbreviated list of cancer types (see FIG. 14 for full set). G. Examples of antigen pairs with improved tumor vs normal discrimination. 2D plots show expression level of two antigens in normal tissue samples (grey) vs specific cancer type samples (red). Navy circles show centroids for each of the normal tissue types (labeled when close to red cancer cluster). These examples are all AND gates. See FIG. 14 for more examples of discriminatory antigen pairs.

FIG. 8A-B depict experimental validation: building and testing combinatorial recognition circuits predicted to enhance Renal Cell Carcinoma discrimination. A. RCC recognition circuit: CD70 and AXL. Segregation of RCC samples (red points) vs normal tissue samples (grey points) in antigen expression space, highlighting overlap of CD70 expression with normal blood samples (green points). An α-AXL synNotch receptor was constructed and the ability of human T cells expressing the receptor to detect 769-P Renal Cell Cancer cell line (CD70⁺AXL⁺), but not Raji B-cell line (CD70⁺AXL⁻) was validated via FAC detection of GFP reporter induction. In cell killing assays, human primary CD8+ T cells constitutively expressing the CD70 CAR were compared with the same cells transfected with the AXL synNotch→CD70 CAR AND-gate circuit. The single antigen targeting CD70 CAR T cells killed both RCC and B-cell lines, while the circuit T cells selectively killed RCC cells. B. RCC recognition circuit: AXL and CDH6. Segregation of RCC samples (red points) vs normal tissue samples (grey points) in antigen expression space, highlighting overlap of AXL expression with normal lung samples (green points). An α-CDH6 synNotch receptor was constructed and the ability of human T cells expressing the receptor to detect 769-P Renal Cell Cancer cell line (AXL⁺CDH6⁺), but not Beas2B lung epithelial cell line (AXL⁺CDH6⁻) was validated via FAC detection of GFP reporter induction. In cell killing assays, human primary CD8+ T cells constitutively expressing the AXL CAR were compared with the same cells transfected with the CDH6 synNotch→AXL CAR AND-gate circuit. The single antigen targeting AXL CAR T cells killed both RCC and lung cell lines, while the circuit T cells selectively killed RCC cells.

FIG. 9A-B depict recognition of AND gate combination antigens in cis or trans (expression on the same or different cells). A. AND-gate “prime-and-kill” synNotch→CAR circuits could in principle be capable of cis- or trans-recognition. In cis-recognition the synNotch and CAR antigens are expressed on the same cell, while in trans-recognition the two antigens are expressed on different cells. The ability of αHER2 synNotch→αCD19 CAR T cells to achieve trans-recognition was tested by co-culturing different mixtures of priming cells (HER2+ CD19-K562 cells) and target cells (HER2-CD19+ K562 cells). B. Prime-and-kill dual-antigen recognition T cells can recognize combination of antigens presented in trans on pancreatic cancer cells and cancer associated stromal cells. Pancreatic cancer samples (red points) in 2D antigen space can be better segregated from normal samples (grey points) through dual recognition of mesothelin (MSLN) and fibroblast activation protein (FAP). MSLN is expressed on the cancer cells, while FAP is expressed on stromal cells (e.g. pancreatic stellate cells—PSC). An αFAP synNotch receptor that detects a PSC cell line was constructed. This receptor was used to engineer a FAP synNotch→MSLN CAR circuit. Human primary CD8+ T cells expressing this circuit were able to efficiently kill PANC04.03 PDAC cells only in the presence of FAP+ PSC cells. Killing was thus far more selective than the constitutive MSLN expressing T cells but was equally efficient.

FIG. 10A-C depict design of new circuits to expand dimensions of combinatorial antigen recognition: combining recognition of internal and external antigens. A. Current CAR T circuits can recognize only 1-2 extracellular antigens while there are, in principle, many more features that could be used for combinatorial recognition, including intracellular antigens (presented on MHC complexes) as well as larger numbers of antigens. B. Combinatorial prime-and-kill AND gate circuits that incorporate internal antigen recognition via three different schemes have been able to be engineered. First, a synNotch receptor (external priming antigen) can be used to induce expression of a TCR (internal killing antigen). Second, an internal synNotch (inNotch) receptor that recognizes a peptideMHC complex (internal priming antigen) can be used to induce expression of a CAR (external killing antigen). Third, an inNotch receptor (internal priming antigen) can be used to induce expression of a TCR (internal killing antigen). The detailed designs and testing of these prototype circuits are shown in FIG. 18D-18H C. Experimental validation of how αMETsynNotch→MART1 TCR circuit can improve discrimination of melanoma (note: MART1 is also referred to as MLANA). A collection of melanoma RNAseq expression data shows that most melanomas (as well as normal primary melanocytes) express MART1, but that a significant subset also express MET. Melanoma cell line M202 is an example of a cancer that is MART1+MET+, while M262 is an example of cancer that is MART1+MET− (both cell lines are HLA2 allowing for recognition by a TCR previously tested in clinical trials—REF). It is found that primary melanocytes are also MART1+MET− (FIG. 18A). An a-MET synNotch receptor was constructed and that T cells expressing this receptor were only activated (BFP reporter) by the M202 MART+MET− cells, and not MET− melanomas or melanocytes was validated. αMETsynNotch→a MART1 TCR circuit was then constructed and its killing specificity is compared to the constitutive a MART1 TCR, when transfected into human primary CD8+ T cells. The MART1 TCR cells are not selective for MET+ vs MET− cells, while the circuit T cells are highly selective, both in killing and activation of T cell proliferation.

FIG. 11A-J depict design of circuits to recognize 3-antigen combinations. A. Two possible schemes for using synNotch and CAR receptors to design 3-antigen AND gate recognition circuits. Both schemes use two synNotch receptors, but akin to electrical circuit, the receptors can be functionally arranged to be in Series or in Parallel. The series 3-input circuit has a first synNotch receptor (recognizes antigen 1), that when activated induces expression of a second synNotch receptor (recognizes antigen 2), which in turn, when activated, induces expression of a CAR (recognizes antigen 3—the killing antigen). In contrast the parallel circuit uses the two different synNotch receptors (recognize antigens 1 and 2) to each induce components of a split CAR, which only when co-expressed will recognize antigen 3 (killing antigen). B. Both possible 3 input AND gate circuits were constructed to recognize the triple antigen combination: EGFR, MET, and HER2. C. Design of the Series 3-input AND gate circuit. In all cases, SynNotch1 recognizes EGFR (using a Gal4 based transcription factor), while synNotch2 recognizes MET (using a LexA transcription factor). In the series circuit, SynNotch receptor 1 regulates expression of synNotch receptor 2, which in turn regulates expression of the Her2 CAR. For design and testing of the Parallel circuit, see FIGS. 19F, 19G, 19D. Testing of recognition by the series synNotch1→synNotch2 cascade to drive mCherry reporter. T cells transfected with this circuit were exposed to K562 cells engineered to express all possible 2 or 3 antigen combinations of EGFR, MET, and HER2. Reporter activation was only observed with target cells expressing all three antigens. E. Selective killing by CD8+ T cells transfected with the Series 3-input AND gate circuit. T cells with the full EGFR synNotch1→MET synNotch2→HER2 CAR circuit only killed K562 cells expressing all three antigens (pink line). No killing was observed of target cells with 2 or fewer of the antigens. F. Selective proliferation of T cells (as in e) only when stimulated with K562 cells expression all three antigens. G. Possible designs of 3-input AND-OR or OR-AND circuits in which tandem CARs or tandem synNotch receptors (each with two antigen recognition domains) can be incorporated to introduce increased recognition flexibility of alternative antigens. H. Scheme for constructing an OR-AND 3 input circuit for (Her2 OR EGFR) AND MET. I. CD8 T cells transfected with the OR circuit (shown in panel i) show efficient killing of target K562 cells that express MET and Her2 or MET and EGFR, but not MET alone. J. sectoring of 3D antigen space in different ways. Example of how 3 antigen space of CD70, AXL, and CDH6 (described as antigen pairs in FIG. 8) is predicted to lead to even better separation of tumor samples from normal tissues (See Supp Movie 1 for rotating 3D plot). The two classes of gates described in this figure sector 3D antigen space in distinct ways. 3-input AND gates select a precise sector of antigen space, while the 3-input AND-OR circuit gives increased flexibility in specific antigen dimensions.

FIG. 12A-B depict a pipeline for computer-aided design of cell therapies: Expansive search of large-scale transcriptomics data for potential cellular circuits to identify optimal tumor vs normal tissue discriminatory opportunities. A. In silico analysis of tumor vs normal expression data can be used to identify discriminatory antigen patterns, and machine learning can be used to match these opportunities with the actionable “periodic table” of synthetic biology recognition circuits. The resulting combinatorial antigen circuits can be experimentally prototyped for disease recognition. Selection of which antigens will serve as priming vs killing antigens is important. B. Common patterns of discriminatory antigen pairs: i) two cancer antigens found in the same cancer, but with non-overlapping normal tissue cross reaction (example: AXL and CD70 in RCC); ii) trans-recognition of cancer antigen (expressed in cancer cells) and priming antigen found in non-cancer cells in the tumor microenvironment. Priming antigens could be on stromal cells (e.g. FAP expression on pancreatic cancer stromal cells) or on tissue specific cells (e.g. MOG expression on brain oligodendrocytes to brain glioblastoma recognition).

FIG. 13 depict enumerating the space of possible multi-antigen recognition circuits. The computational analysis suggests that 2-3 antigen combinations may be sufficient to achieve near ideal discrimination of most cancer types. If the modes of antigen recognition that are possible are considered, it is predicted from first principles that there will be finite “periodic table” of possible combinatorial recognition circuits for 2-3 antigens, each that can section antigen space in distinct ways. Here all of these predicted possible recognition circuits, and how they could be constructed from modular receptors (CARs, TCRs, synNotch receptors, inNotch receptors, inhibitory CARs) are outlined. Alternative mechanisms for constructing such recognition circuits are possible, especially with the future development of alternative sensors, but the basic classes of circuits are unlikely to change significantly.

FIG. 14A-C depict A. To calculate F1 scores for a particular antigen pair the set of data was first divided up into training and test partitions using geometric sketching by Hie et al. 2019 [PMID: 31176620], keeping 20% of the data for the training set and the remaining 80% for evaluation. When then fit decision tree classifiers to the training data to draw a decision boundary separating the tumor samples from all normal tissue samples. The samples in the test set when the labeled using this decision boundary from which when then calculated performance based on known labels. B. Cartoon depicting three possible cases and corresponding F1 scores. C. Table of antigens referred to as clinical in this manuscript and their corresponding clinical trial identifier.

FIG. 15A-C depict A. More example scatterplots of different types of antigen pairs. B. The effect of subsampling on runtime, Davies-Bouldin, and manhattan distance calculations shown for one tumor type, Uveal Melanoma. The two components of the distance based scores, Davies-Bouldin and manhattan distance, were calculated for 8 different sketch sizes for 5 iterations across 100 gene pairs (10 fixed genes paired with 10 random genes). The first chart shows runtime as a function of subset size, and it is seen that runtime using a sketch size of 20% gives substantial improvement when calculating the distance scores on many pairs. The remaining charts show the spearman correlation between the scores calculated on sketches of the data vs. using the entire dataset. Grey shading shows the standard deviation calculated from the 5 iterations. Taken together, using 20% of the data is sufficient to calculate highly concordant distance scores with substantial speed improvements. C. The total number of potential pairs per cancer type with clustering-based scores greater than or equal to 0.85.

FIG. 16A-C depict A. Pie chart showing the composition of different gate types (high:high, high:low) of pairs in the top 10 per tumor type. B. Examples of some high:low pairs to be used in an AND NOT gating framework. C. Improvement over single clinical antigen targets for the full set of tumor types in TCGA. Shown here is the best pair for each tumor type and each best performing clinical as selected in the top 10 scores per tumor type. Any of the pairs of antigens shown in C can be used in a circuit, as described herein. For example, the pairs may represent the first and second synNotches, or the pairs could be combined with another antigen if the circuit contains two synNotches and a CAR.

FIG. 17A-C depict A. Flow cytometry plots showing the expression levels of Axl and CD70 on target cells utilized for experiments comparing the specificity of killing by T cells engineered with a constitutive α-CD70 CAR or an αAxl Gal4-VP64 synNotch→αCD70 CAR circuit. The Raji B cell line is CD70+/Axl−, while the 769-P renal cell cancer cell line is CD70+/Axl+. B. Flow cytometry plots showing the expression levels of CDH6 and Axl on target cells utilized for experiments comparing the specificity of killing by T cells engineered with a constitutive α-Axl CAR or an αCDH6 Gal4-VP64 synNotch→αAxl CAR circuit. The Beas2B lung epithelial cell line is Axl+/CDH6−, while the 769-P renal cell cancer cell line is Axl+/CDH6+. C. Flow cytometry plots showing the expression levels of FAP and Msln on target cells utilized for experiments comparing the specificity of killing by T cells engineered with a constitutive α-Msln CAR or an αFAP Gal4-VP64 synNotch→αMsln CAR circuit.

FIG. 18A-H depict A. Flow cytometry plots showing the expression levels of MET and MART1 on target cells utilized for experiments comparing the specificity of killing by T cells engineered with a constitutive α-MART1 TCR or an αMET Gal4-VP64 synNotch→αMART1 TCR circuit. Both primary human melanocytes and the melanoma cell line M262 are MART1+/MET−, while the melanoma cell line M202 is MART1+/MET+. B. Primary human CD8 T cells were engineered with a constitutive α-MART1 TCR or an αMET Gal4-VP64 synNotch→αMART1 TCR circuit. These T cells were labelled with CellTrace FAR Red and co-cultured at 1:1 E:T for varying times with primary human melanocytes that were HLA-A2+/MART1+/MET−. The supernatant from these co-cultures was collected at 24 hours and analyzed for IFNγ levels via an ELISA. After 5 days of co-culture remaining cells were collected and T cell proliferation was determined by measuring CellTrace dye dilution via flow cytometry. Both cytokine and proliferation readouts showed that only constitutive α-MART1 TCR T cells activated in response to the primary human melanocytes, while synNotch→TCR circuit T cells showed no activation (Cytokine: n=3, error bars are SD; Proliferation: representative of 3 independent experiments). C. Primary human CD8 T cells were engineered as in FIG. 18B, with untransduced T cells serving as a negative control for target cell killing. These T cells were co-cultured for 96 hours with primary human melanocytes that had been labelled with CellTrace FAR Red to distinguish them from the T cells. Confocal microscopy images were taken immediately after and 96 hours after T cells were added to the wells. Representative images show that constitutive α-MART1 TCR T cells cleared the primary melanocytes and clustered as typical with T cell activation, while neither untransduced or synNotch→TCR circuit T cells showed any reactivity. D. Design of a dual antigen AND gate circuit controlling T cell activation that recognizes an internal-external antigen pair: the α-AFP Gal4-VP64 inNotch (internal, pMHC) induces expression of the α-Her2 41BBζ CAR (external). E. Primary human CD8 T cells were engineered to express an internal antigen-sensing synNotch (“inNotch”) targeting an MHC-presented peptide from the cytosolic/secreted protein AFP and utilizing Gal4-VP64 to control expression of a corresponding BFP reporter response element. These T cells were co-cultured at 1:1 E:T for 24 hours with T2 target cells pulsed with a titration of AFP₁₅₈₋₁₆₆ peptide or off-target WT1 peptide. Histograms of BFP levels measured via flow cytometry show selective reporter induction in the presence of AFP, with increasing induction in response to increasing AFP peptide levels (representative of 3 independent experiments). F. Engineered CD8 T cells described in FIG. 18D were co-cultured at 1:1 E:T for 24 hours with T2 target cells presenting AFP only, Her2 only, or both AFP and Her2. Target cell survival was determined via flow cytometry, which demonstrated that inNotch→CAR AND gate T cells specifically killed the dual-positive AFP+/Her2+ T2 cells (n=3, error bars are SD). G. Design of a dual antigen AND gate circuit controlling T cell activation that recognizes an internal-internal antigen pair: the α-AFP Gal4-VP64 inNotch (internal, pMHC) induces expression of the α-NY-ESO1 TCR (internal, pMHC). H. Engineered CD8 T cells described in FIG. 18G were co-cultured at 1:1 E:T for 24 hours with T2 target cells presenting AFP only, NY-ESO1 only, or both AFP and NY-ESO1. Target cell survival was determined via flow cytometry, which demonstrated that inNotch→TCR AND gate T cells specifically killed the dual-positive AFP+/NY-ESO1+ T2 cells (n=3, error bars are SD).

FIG. 19A-G depict A. Diagram depicting CD4 T cells engineered to test orthogonality of multiple synNotch receptors in the same primary human T cell. The T cells were engineered to express (1) an α-GFP synNotch receptor with a LexA-VP64 transcription factor controlling mCherry reporter expression and (2) an α-Her2 synNotch receptor with a Gal4-VP64 transcription factor controlling BFP reporter expression. B. Engineered T cells shown in (A) were co-cultured at 1:1 E:T for 24 hours with K562 target cells expressing GFP, Her2, neither, or both antigens. Bar graphs showing the integrated fluorescence signal for both mCherry and BFP reporter outputs demonstrate that the LexA- and Gal4-based synNotch receptors function orthogonally in primary human T cells (n=2, error bars are SD). C. Flow cytometry plots showing the expression levels of MET, EGFR, and Her2 on K562 lines utilized for experiments with 3-input AND-gate circuit T cells. D. Primary human CD8 T cells were engineered with a constitutive α-Her2 41BBζ CAR to use as a positive control in target cell killing assays for T cells engineered with the full EGFR synNotch1→MET synNotch2→HER2 CAR 3-input AND-gate In-series circuit. E. Engineered T cells shown in FIG. 19D were co-cultured at 1:1 E:T for varying times with K562 target cells expressing different combinations of Her2, EGFR, and MET, and target cell survival was determined via flow cytometry. Unlike the 3-input AND-gate In-series T cells which selectively killed triple positive target cells, constitutive α-Her2 CAR T cells killed any K562 cell line that expressed Her2, regardless of EGFR and MET expression (n=3, error bars are SD). F. Diagram depicting 3-receptor circuit engineered in primary human CD8 T cells to generate a 3-input AND gate In-parallel circuit controlling T cell activation. Multiple orthogonal synNotch receptors are expressed in CD8 T cells, each controlling expression of a different part of a split CAR. The split CAR is made up of the α-PNE 41BBζ CAR (CARᵢₙ) and secreted α-Her2 scFv linked to the PNE peptide (CARₒᵤₜ). The engineered CD8 T cells can recognize and kill Her2+ target cells only when both parts of the split CAR are expressed. The α-EGFR Gal4-VP64 synNotch controls expression of the CARᵢₙ and the α-MET LexA-VP64 synNotch controls expression of the CARₒᵤₜ. G. Engineered T cells shown in FIG. 19F were co-cultured at 1:1 E:T for varying times with K562 target cells expressing different combinations of Her2, EGFR, and MET, and target cell survival was determined via flow cytometry. Unlike the 3-input AND-gate In-series T cells which selectively killed triple positive target cells, the 3-input AND-gate In-parallel T cells were less specific and also strongly killed dual positive MET+/Her2+ target cells (n=3, error bars are SD).

FIG. 20A-D depict A. Flow cytometry plots showing the expression levels of MET, EGFR, and Her2 on K562 lines utilized for experiments with OR-AND 3-input circuit T cells. B. Primary human CD8 T cells were engineered with an α-EGFR synNotch, α-Her2 synNotch, or α-Her2/EGFR tandem synNotch controlling the expression of a BFP reporter. All synNotch receptors utilized a Gal4-VP64 transcription factor These T cells were co-cultured at 1:1 E:T for 24 hours with K562 target cells expressing Her2, EGFR, or neither antigen. Histograms of BFP levels measured via flow cytometry show that the dual synNotch specifically responds to either Her2 OR EGFR positive target cells, while the two single antigen synNotch receptors only respond to one of the target cells (representative of 3 independent experiments). C. Primary human CD8 T cells were engineered with a constitutive α-MET 41BBζ CAR to use as a positive control in target cell killing assays for T cells engineered with the OR-AND 3-input circuit for (Her2 OR EGFR) AND MET. D. Engineered T cells shown in FIG. 20C were co-cultured at 1:1 E:T for 72 hours with K562 target cells expressing different combinations of MET, EGFR, and Her2, and target cell survival was determined via flow cytometry. Unlike the OR-AND 3-input circuit T cells which selectively killed MET+ K562s that also expressed EGFR or Her2, constitutive α-MET CAR T cells killed any K562 cell line that expressed MET, regardless of EGFR and Her2 expression (n=3, error bars are SD).

### DEFINITIONS

As used herein, the term “heterogeneous”, when used in reference to cancer, generally refers to a cancer displaying some level of intracancer or intratumor heterogeneity, e.g., at the molecular, cellular, tissue or organ level. A heterogeneous cancer is composed of at least two different cell types, where different cell types may be defined in variety of ways. For example, different cell types may differ genomically (e.g., through the presence of a mutation in one cell type that is absent in another), transcriptionally (e.g., through expression of a gene in one cell type that is not expressed in another, through enhanced or reduced expression of a gene in one cell type as compared to another, etc.), or proteomically (e.g., through expression of a protein in one cell type that is not expressed in another, through enhanced or reduced expression of a protein in one cell type as compared to another, etc.). In some instances, cancer heterogeneity may be identified based on the presence of two or more phenotypically different cells present in a cancer, including e.g., where such phenotypically different cells are identified through clinical testing (e.g., histology, immunohistochemistry, in situ hybridization, cytometry, transcriptomics, mutational analysis, whole genome sequencing, proteomics, etc.).

As such, a heterogeneous cancer, as defined herein, will generally include at least one cancerous cell type and at least one other cell type, where the one other cell type may be a second cancerous cell type or a non-cancerous cell type. For example, a heterogeneous cancer may include a first cancerous cell type and a second cancerous cell type. Alternatively, a heterogeneous cancer may include a cancerous cell type and a non-cancerous cell type. Although a heterogeneous cancer will include at least two different cell types, such cancers are not so limited and may include e.g., more than two different cell types, three or more different cell types, four or more different cell types, five or more different cell types, etc., where at least one cell type is cancerous and the additional cell types may each be cancerous or non-cancerous.

As summarized above, heterogeneity of a cancer may be defined by differing gene or protein expression by different subpopulations of cells of the cancer. For example, in some instances, a first subpopulation of cells may express a first gene product from a first gene that is not expressed by a second subpopulation of cells, where such a second cell population may or may not express a second gene product from a second gene that defines the second population. Put another way, subpopulations of cells within a heterogeneous cancer may, in some instances, each be defined by the presence or absence (or relative levels) of one or more expressed gene products, where useful expressed gene products for defining cell types may include but are not limited to biomarkers, antigens, wild-type proteins, mutated proteins, wild-type transcripts, mutated transcripts, etc.

Cancer heterogeneity, in some instances, may include or exclude heterogeneity at the subject level, i.e., intrapatient heterogeneity. As used herein, the term “intrapatient heterogeneity” generally refers to heterogeneity observed between multiple cancers, e.g., multiple tumors, present in a single subject. For example, a primary tumor and a metastasis with a subject may be heterogeneous, e.g., differentially expressing a particular gene product, such as a biomarker, an antigen or a mutated protein. Multiple heterogeneous cancers may arise in a subject through various mechanisms including but not limited to mutation, clonal expansion, metastasis, selection, and combinations thereof. For example, two different intrapatient heterogeneous cancers arising by metastasis of a primary tumor may be heterogeneous with respect to the tissues in which they reside. Alternatively, two different intrapatient heterogeneous cancers derived from the same primary tumor may arise due to mutation and clonal expansion, where one cancer is a subclone of the other. Various other mechanism by which different intrapatient heterogeneous cancers may arise are possible and fall within the scope of the term as used herein.

Cancer heterogeneity, in some instances as used herein, may exclude heterogeneity at the population level, i.e., interpatient heterogeneity. As used herein, the term “interpatient heterogeneity” generally refers to differences observed between two cancers or two tumors present in separate subjects or patients.

As used herein, the terms “treatment,” “treating,” “treat” and the like, refer to obtaining a desired pharmacologic and/or physiologic effect and/or a response related to the treatment. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

A “therapeutically effective amount” or “efficacious amount” refers to the amount of an agent (including biologic agents, such as cells), or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

The terms “individual,” “subject,” “host,” and “patient,” used interchangeably herein, refer to a mammal, including, but not limited to, murines (e.g., rats, mice), non-human primates, humans, canines, felines, ungulates (e.g., equines, bovines, ovines, porcines, caprines), lagomorphs, etc. In some cases, the individual is a human. In some cases, the individual is a non-human primate. In some cases, the individual is a rodent, e.g., a rat or a mouse. In some cases, the individual is a lagomorph, e.g., a rabbit.

The term “refractory”, used herein, refers to a disease or condition that does not respond to treatment. With regard to cancer, “refractory cancer”, as used herein, refers to cancer that does not respond to treatment. A refractory cancer may be resistant at the beginning of treatment or it may become resistant during treatment. Refractory cancer may also called resistant cancer.

The term “histology” and “histological” as used herein generally refers to microscopic analysis of the cellular anatomy and/or morphology of cells obtained from a multicellular organism including but not limited to plants and animals.

The term “cytology” and “cytological” as used herein generally refers to a subclass of histology that includes the microscopic analysis of individual cells, dissociated cells, loose cells, clusters of cells, etc. Cells of a cytological sample may be cells in or obtained from one or more bodily fluids or cells obtained from a tissue that have been dissociated into a liquid cellular sample.

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled (e.g., as described in PCT publication no. WO 2014/127261 A1 and US Patent Application No. 2015/0368342 A1, the disclosures of which are incorporated herein by reference in their entirety). CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety. Useful CARs also include the anti-CD19—4-1BB—CD3ζ CAR expressed by lentivirus loaded CTL019 (Tisagenlecleucel-T) CAR-T cells as commercialized by Novartis (Basel, Switzerland).

The terms “T cell receptor” and “TCR” are used interchangeably and will generally refer to a molecule found on the surface of T cells, or T lymphocytes, that is responsible for recognizing fragments of antigen as peptides bound to major histocompatibility complex (MHC) molecules. The TCR complex is a disulfide-linked membrane-anchored heterodimeric protein normally consisting of the highly variable alpha (α) and beta (β) chains expressed as part of a complex with CD3 chain molecules. Many native TCRs exist in heterodimeric αβ or γβ forms. The complete endogenous TCR complex in heterodimeric αβ form includes eight chains, namely an alpha chain (referred to herein as TCRα or TCR alpha), beta chain (referred to herein as TCRβ or TCR beta), delta chain, gamma chain, two epsilon chains and two zeta chains. In some instance, a TCR is generally referred to by reference to only the TCRα and TCRβ chains, however, as the assembled TCR complex may associate with endogenous delta, gamma, epsilon and/or zeta chains an ordinary skilled artisan will readily understand that reference to a TCR as present in a cell membrane may include reference to the fully or partially assembled TCR complex as appropriate.

Recombinant or engineered individual TCR chains and TCR complexes have been developed. References to the use of a TCR in a therapeutic context may refer to individual recombinant TCR chains. As such, engineered TCRs may include individual modified TCRα or modified TCRβ chains as well as single chain TCRs that include modified and/or unmodified TCRα and TCRβ chains that are joined into a single polypeptide by way of a linking polypeptide.

As used herein, by “chimeric bispecific binding member” is meant a chimeric polypeptide having dual specificity to two different binding partners (e.g., two different antigens). Non-limiting examples of chimeric bispecific binding members include bispecific antibodies, bispecific conjugated monoclonal antibodies (mab)2, bispecific antibody fragments (e.g., F(ab)₂, bispecific scFv, bispecific diabodies, single chain bispecific diabodies, etc.), bispecific T cell engagers (BiTE), bispecific conjugated single domain antibodies, micabodies and mutants thereof, and the like. Non-limiting examples of chimeric bispecific binding members also include those chimeric bispecific agents described in Kontermann. MAbs. (2012) 4(2): 182-197; Stamova et al. Antibodies 2012, 1(2), 172-198; Farhadfar et al. Leuk Res. (2016) 49:13-21; Benjamin et al. Ther Adv Hematol. (2016) 7(3):142-56; Kiefer et al. Immunol Rev. (2016) 270(1):178-92; Fan et al. J Hematol Oncol. (2015) 8:130; May et al. Am J Health Syst Pharm. (2016) 73(1):e6-e13; the disclosures of which are incorporated herein by reference in their entirety.

As used herein, the term “binding-triggered transcriptional switch” or “BTTS” refers to any polypeptide or complex of the same that is capably of transducing a specific binding event on the outside of the cell (e.g. binding of an extracellular domain of the BTTS) to activation of a recombinant promoter within the nucleus of the cell. Many BTTSs work by releasing a transcription factor that activates the promoter. In these embodiments, the BTTS is made up of one or more polypeptides that undergo proteolytic cleavage upon binding to the antigen to release a gene expression regulator that activates the recombinant promoter. For example, a BTTS may comprise (i) an extracellular domain comprising the antigen binding region of a antigen-specific antibody; (ii) a proteolytically cleavable sequence comprising one or more proteolytic cleavage sites; and (iii) an intracellular domain, wherein binding of the antigen binding region to the antigen induces cleavage of the sequence at the one or more proteolytic cleavage sites, thereby releasing the intracellular domain and wherein the intracellular domain activates transcription of an expression cassette. A BTTS can be based on synNotch, A2, MESA, or force receptor, for example, although others are known or could be constructed.

A “biological sample” encompasses a variety of sample types obtained from an individual or a population of individuals and can be used in various ways, including e.g., the isolation of cells or biological molecules, diagnostic assays, etc. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by mixing or pooling of individual samples, treatment with reagents, solubilization, or enrichment for certain components, such as cells, polynucleotides, polypeptides, etc. The term “biological sample” encompasses a clinical sample, and also includes cells in culture, cell supernatants, cell lysates, serum, plasma, biological fluid, and tissue samples. The term “biological sample” includes urine, saliva, cerebrospinal fluid, interstitial fluid, ocular fluid, synovial fluid, blood fractions such as plasma and serum, and the like. The term “biological sample” also includes solid tissue samples, tissue culture samples (e.g., biopsy samples), and cellular samples. Accordingly, biological samples may be cellular samples or acellular samples.

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, nanobodies, single-domain antibodies, and fusion proteins comprising an antigen-binding portion of an antibody and a non-antibody protein.

“Antibody fragments” comprise a portion of an intact antibody, for example, the antigen binding or variable region of the intact antibody. Examples of antibody fragments include Fab, Fab′, F(ab′)2, and Fv fragments; diabodies; linear antibodies (Zapata et al., Protein Eng. 8(10): 1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

“Single-chain Fv” or “sFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids (Hamers-Casterman et al. (1993) Nature 363:446; Desmyter et al. (2015) Curr. Opin. Struct. Biol. 32:1). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody.

As used herein, the term “affinity” refers to the equilibrium constant for the reversible binding of two agents and is expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. Non-specific binding would refer to binding with an affinity of less than about 10⁻⁷ M, e.g., binding with an affinity of 10⁻⁶ M, 10⁻⁵ M, 10⁻⁴ M, etc.

A “orthogonal” or “orthogonalized” member or members of a binding pair are modified from their original or wild-type forms such that the orthogonal pair specifically bind one another but do not specifically or substantially bind the non-modified or wild-type components of the pair. Any binding partner/specific binding pair may be orthogonalized, including but not limited to e.g., those binding partner/specific binding pairs described herein.

The terms “domain” and “motif”, used interchangeably herein, refer to both structured domains having one or more particular functions and unstructured segments of a polypeptide that, although unstructured, retain one or more particular functions. For example, a structured domain may encompass but is not limited to a continuous or discontinuous plurality of amino acids, or portions thereof, in a folded polypeptide that comprise a three-dimensional structure which contributes to a particular function of the polypeptide. In other instances, a domain may include an unstructured segment of a polypeptide comprising a plurality of two or more amino acids, or portions thereof, that maintains a particular function of the polypeptide unfolded or disordered. Also encompassed within this definition are domains that may be disordered or unstructured but become structured or ordered upon association with a target or binding partner. Non-limiting examples of intrinsically unstructured domains and domains of intrinsically unstructured proteins are described, e.g., in Dyson & Wright. Nature Reviews Molecular Cell Biology 6:197-208.

The terms “synthetic”, “chimeric” and “engineered” as used herein generally refer to artificially derived polypeptides or polypeptide encoding nucleic acids that are not naturally occurring. Synthetic polypeptides and/or nucleic acids may be assembled de novo from basic subunits including, e.g., single amino acids, single nucleotides, etc., or may be derived from pre-existing polypeptides or polynucleotides, whether naturally or artificially derived, e.g., as through recombinant methods. Chimeric and engineered polypeptides or polypeptide encoding nucleic acids will generally be constructed by the combination, joining or fusing of two or more different polypeptides or polypeptide encoding nucleic acids or polypeptide domains or polypeptide domain encoding nucleic acids. Chimeric and engineered polypeptides or polypeptide encoding nucleic acids include where two or more polypeptide or nucleic acid “parts” that are joined are derived from different proteins (or nucleic acids that encode different proteins) as well as where the joined parts include different regions of the same protein (or nucleic acid encoding a protein) but the parts are joined in a way that does not occur naturally.

The term “recombinant”, as used herein describes a nucleic acid molecule, e.g., a polynucleotide of genomic, cDNA, viral, semisynthetic, and/or synthetic origin, which, by virtue of its origin or manipulation, is not associated with all or a portion of the polynucleotide sequences with which it is associated in nature. The term recombinant as used with respect to a protein or polypeptide means a polypeptide produced by expression from a recombinant polynucleotide. The term recombinant as used with respect to a host cell or a virus means a host cell or virus into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

The term “operably linked” refers to a juxtaposition wherein the components so described are in a relationship permitting them to function in their intended manner. For instance, a promoter is operably linked to a coding sequence if the promoter affects its transcription or expression. Operably linked nucleic acid sequences may but need not necessarily be adjacent. For example, in some instances a coding sequence operably linked to a promoter may be adjacent to the promoter. In some instances, a coding sequence operably linked to a promoter may be separated by one or more intervening sequences, including coding and non-coding sequences. Also, in some instances, more than two sequences may be operably linked including but not limited to e.g., where two or more coding sequences are operably linked to a single promoter.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

A “vector” or “expression vector” is a replicon, such as plasmid, phage, virus, or cosmid, to which another DNA segment, i.e. an “insert”, may be attached so as to bring about the replication of the attached segment in a cell.

An “expression cassette” has at least a coding sequence, a promoter, and terminator, where those components are operably linked.

The term “heterologous”, as used herein, means a nucleotide or polypeptide sequence that is not found in the native (e.g., naturally-occurring) nucleic acid or protein, respectively. Heterologous nucleic acids or polypeptide may be derived from a different species as the organism or cell within which the nucleic acid or polypeptide is present or is expressed. Accordingly, a heterologous nucleic acids or polypeptide is generally of unlike evolutionary origin as compared to the cell or organism in which it resides.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cell” includes a plurality of such cells and reference to “the cell” includes reference to one or more cells and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

As noted above, this disclosure provides an in-series synthetic receptor circuit for dual-antigen AND-gate control over expression of a therapeutic payload by engineered cells. In some embodiments, the circuit may be composed of a first synNotch, a second synNotch and a therapeutic payload (e.g., a chimeric antigen receptor), where binding of the first synNotch to a first antigen activates expression of the second synNotch, and binding of the second synNotch to a second antigen activates expression of the therapeutic payload. If the cell is an immune cell and the therapeutic payload is a chimeric antigen receptor, then the immune cell may be activated by binding of the chimeric antigen receptor to a third antigen. In these embodiments, the first, second and third antigens may be expressed on the surface of the same cell (e.g., a cancer cell) and, as such, some embodiments of the circuit may require a minimum of three antigens for activation.

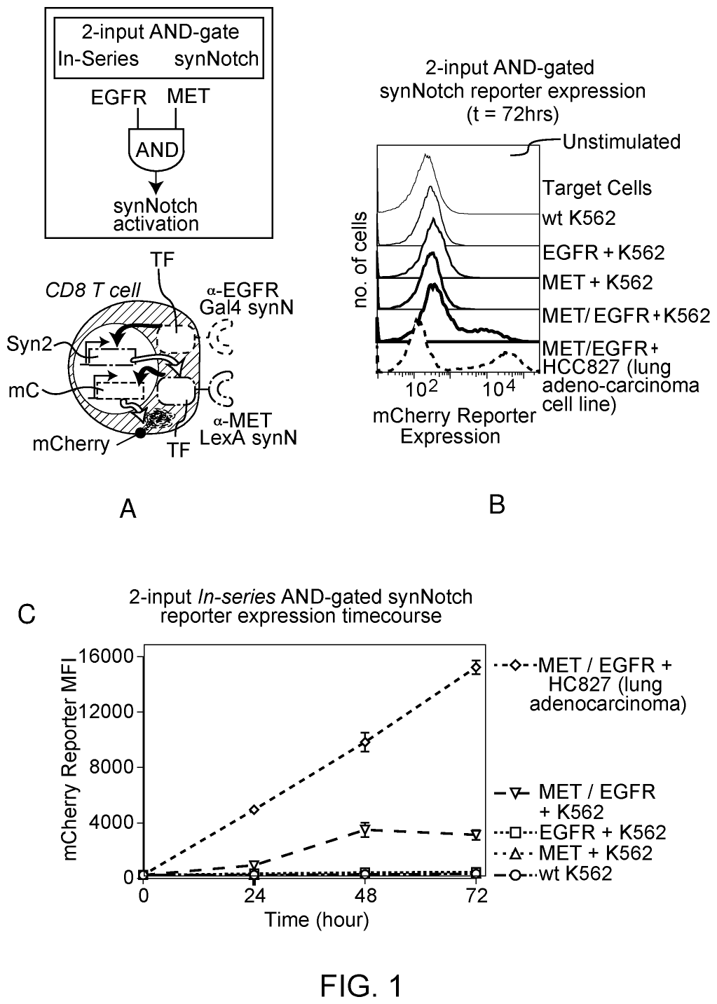

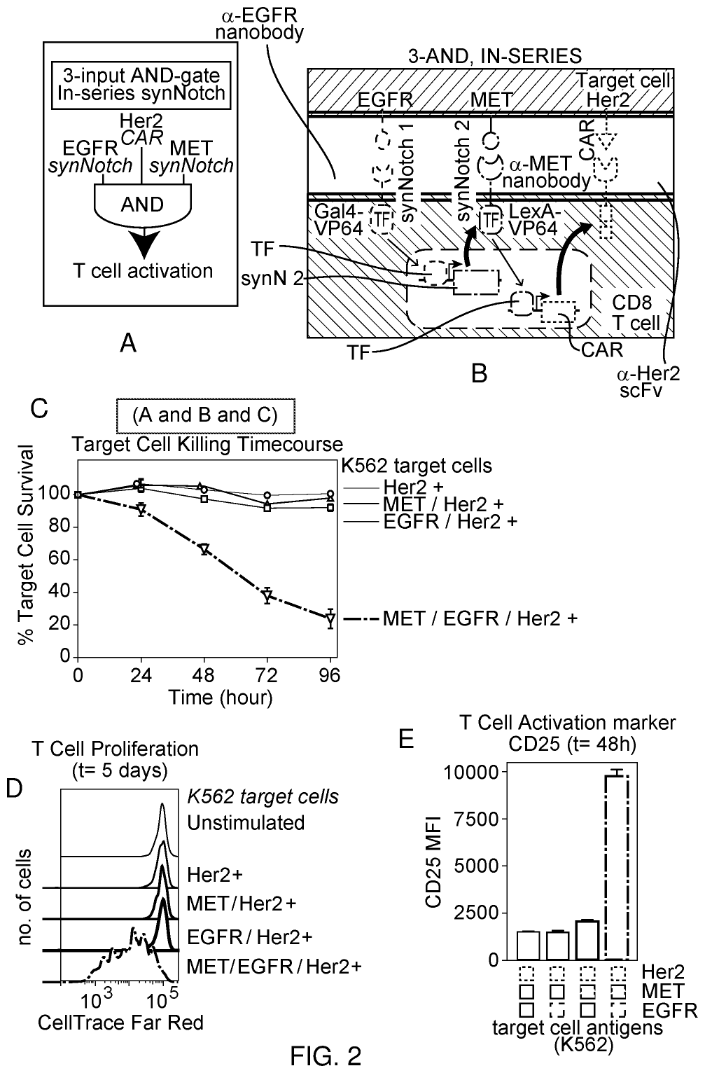

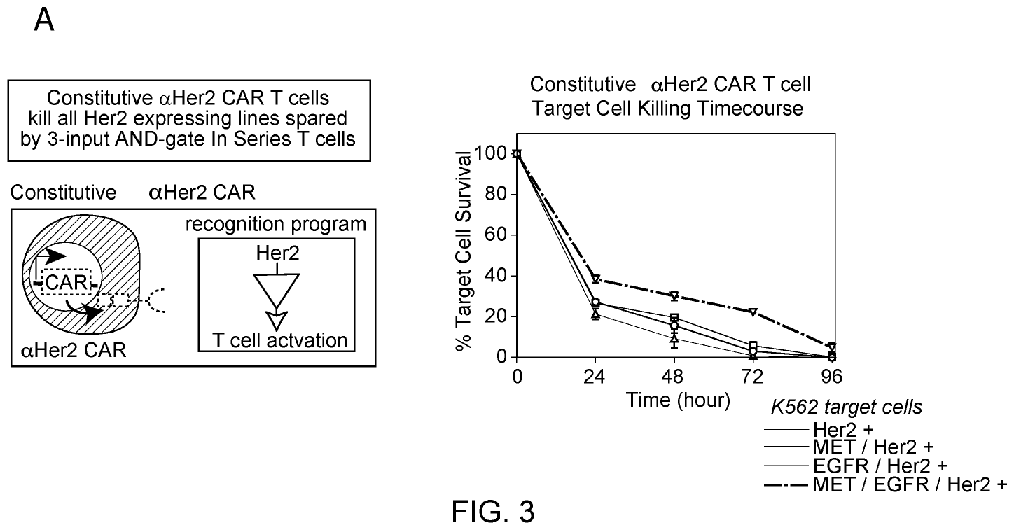

As described in the examples section of this disclosure, to prototype three-input AND gates, the combination of tumor antigens EGFR, MET, and HER2 were targeted. For the in-series AND gate, a circuit in which an anti-EGFR synNotch receptor induced expression of an anti-Met synNotch receptor, which in turn induced expression of an anti-Her2 CAR, was designed. Here the two synNotch receptors use orthogonal transcriptional domains to avoid cross-talk. It was first shown that T cells engineered with an anti-EGFR Gal4-VP64 synNotch In-Series with an anti-MET LexA-VP64 controlling mCherry expression only activated the fluorescent reporter in response to EGFR+/MET+ K562 or HCC827 target cells, but not cells expressing either single antigen (FIG. 1). As mCherry is a proxy for a therapeutic payload, this demonstrates the potential of In-Series synNotch circuits to control the expression of diverse biologic payloads. Next expression of an anti-Her2 CAR was put under control of a EGFR→MET In-Series synNotch circuit, and it was found that T cells engineered with this circuit selectively were activated by and killed MET+/EGFR+/Her2+ K562 target cells, while sparing cells expressing Her2 alone or with only one of the two other antigens in the circuit (FIG. 2). The highly specific killed by the 3-input in-series circuit T cells was in contrast to the indiscriminant killing of all Her2+ cells by constitutive anti-Her2 CAR T cells (FIG. 3).

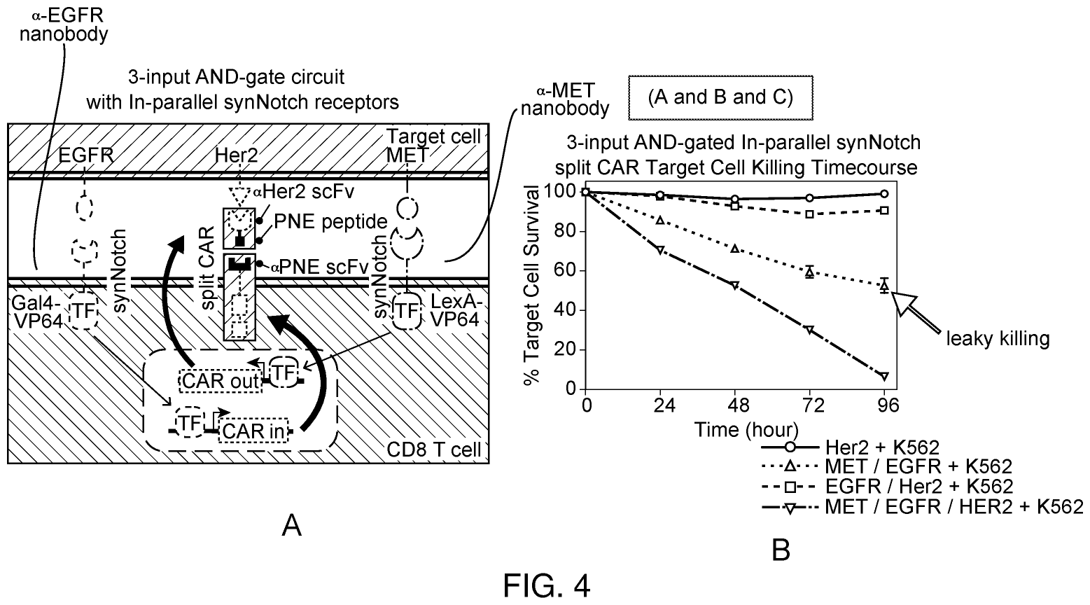

An analogous in-parallel three input AND gate, using the same antigen targets, was also constructed but in this case the EGFR and MET synNotch receptors were used to drive expression of separate components of a split a-Her2 CAR (the CAR requires a Her2 scFv-peptide fusion as an adapter). Testing of this Parallel circuit is shown in FIG. 4. This circuit enhanced specificity for target cells expressing all three target antigens, but showed significantly increased leakiness compared to the in Series circuit—moderate levels of killing were observed for cells expressing only two of three antigens.

Overall, construction of the three-input gates is more straightforward since it take advantage of the modularity of synNotch receptors. The in-Series scheme leads to the most stringent control of killing activity, while still maintaining effective killing. The significant improvement in targeting specificity seen with the in-Series 3-input AND-gate versus the in-parallel version was not expected before building and testing the two systems.

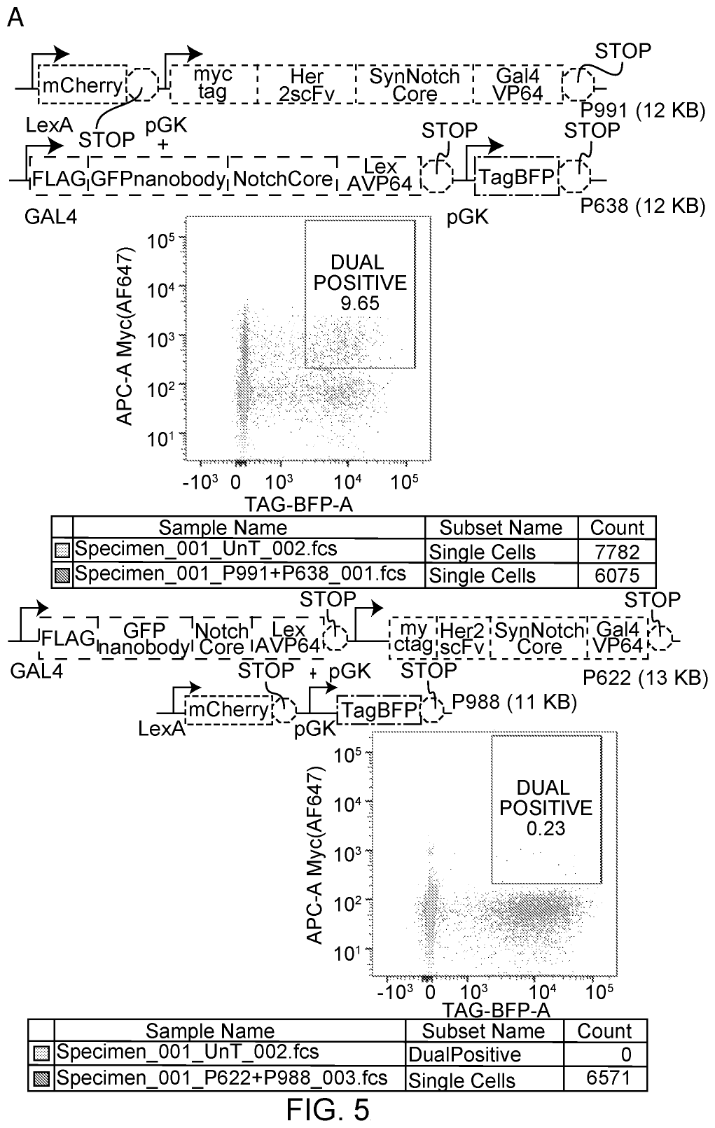

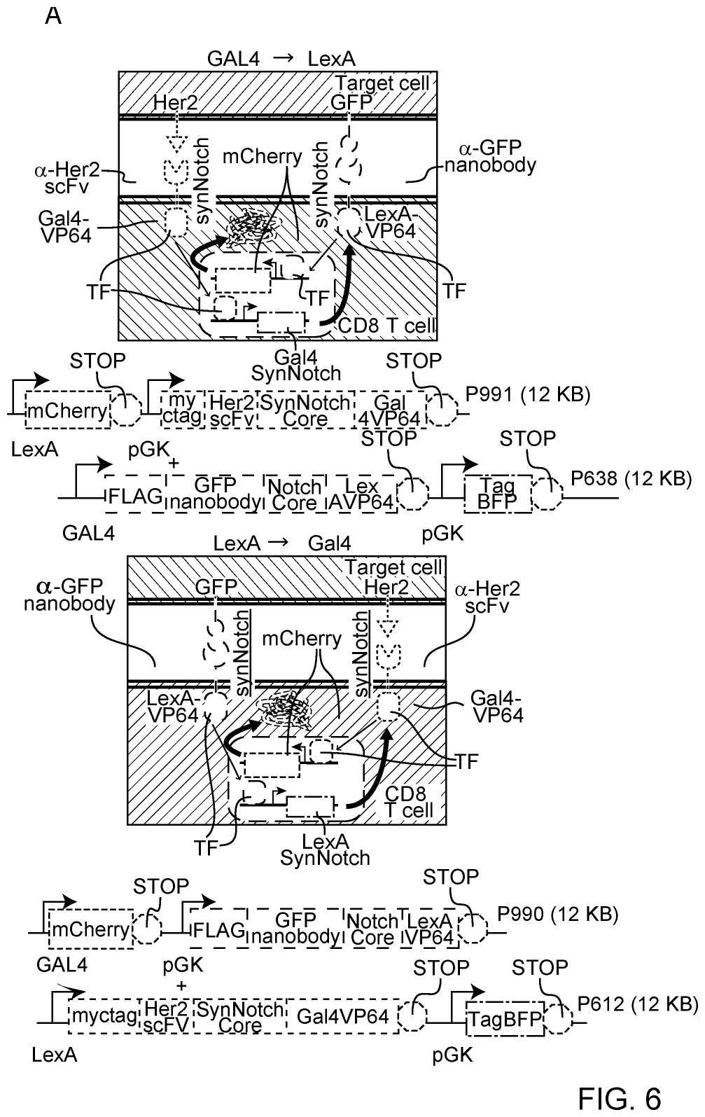

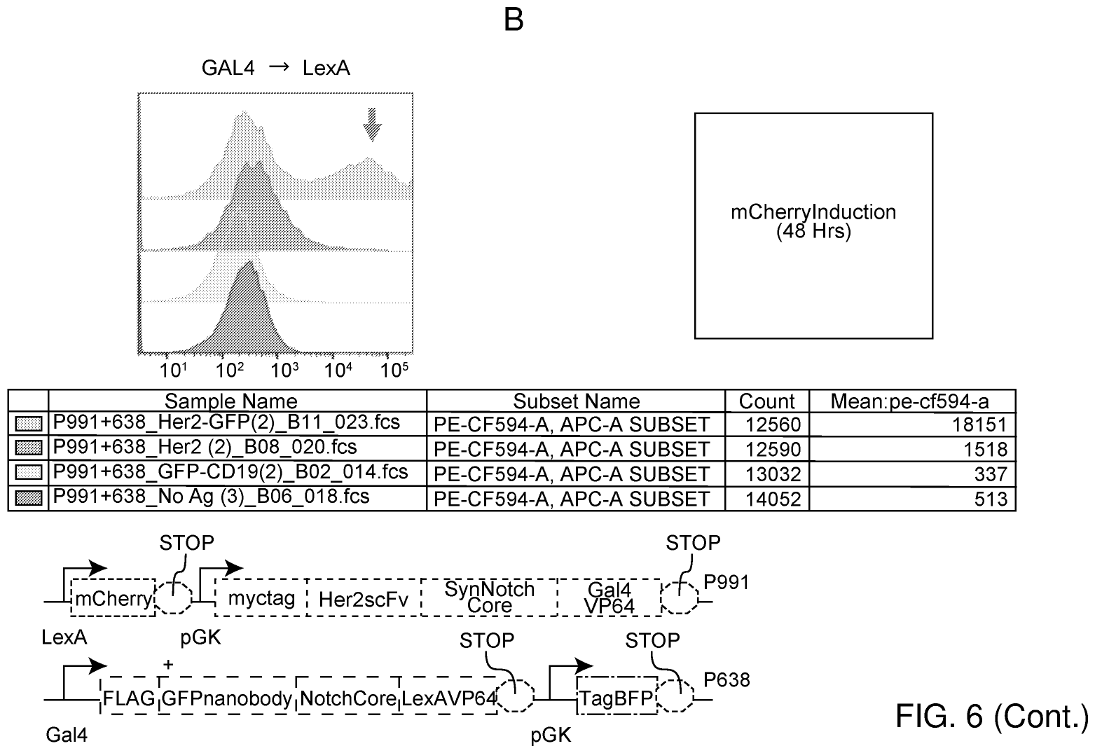

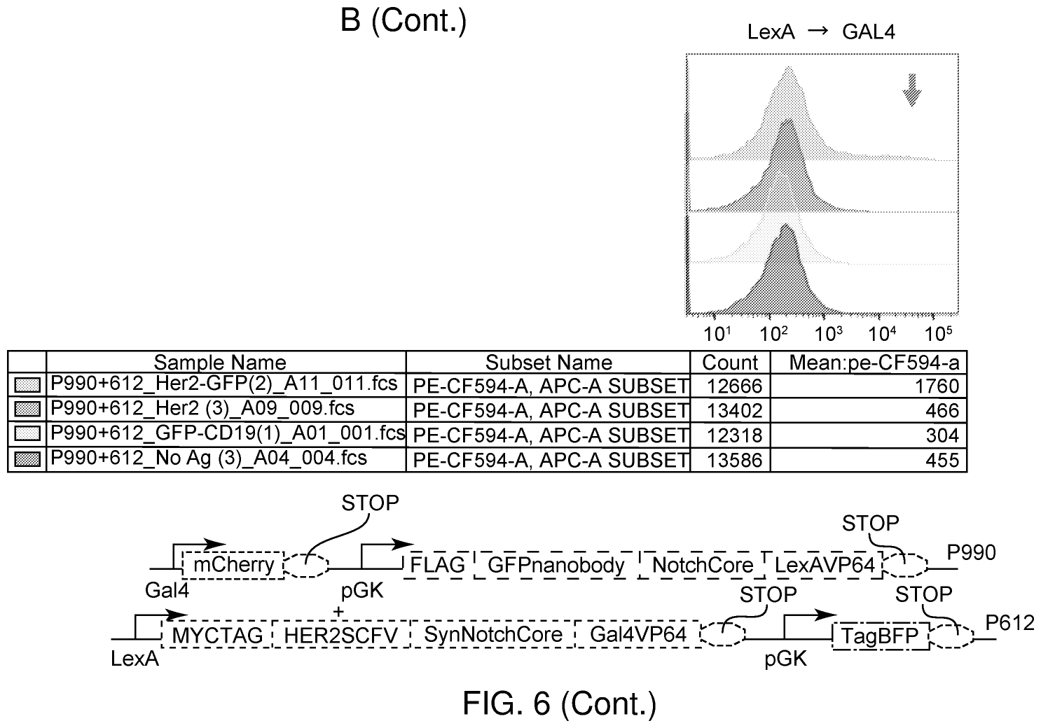

In building and testing in-series BTTS/synNotch circuits, a few improvements were identified. First, it was found that the arrangement of the various genetic elements that make up the circuit on two separate plasmids made a significant difference in the ability to express the circuit in primary human T cells (FIG. 5). Analyzing expression of the constitutively expressed elements of both plasmids making up this exemplary in-series synNotch circuits revealed that the plasmid with the constitutively expressed and inducible synNotch receptors did not express (FIG. 5, right panel). In contrast, when the constitutively expressed and inducible synNotch receptors were divided between the two plasmids T cells could successfully be co-transduced with both plasmids. Thus, in-series synNotch circuits used particular plasmid configurations to be expressed in primary human T cells. Second, it was found that the order of the two synNotch receptors in the in-series circuit may be important when the two orthogonal synthetic transcription factors have different strengths (FIG. 6). In this example, synNotch receptors that use Gal4-VP64 transcription factors are able to drive stronger responses than synNotch receptors that use LexA-VP64. The results show that having the synNotch with the stronger transcription factor (Gal4-VP64) upstream of the synNotch with the weaker transcription factor (LexA-VP64) was mCherry induction more strongly in the presence of both synNotch antigens (FIG. 6). Therefore, in-series circuits should be built with the BTTS capable of driving the stronger response as the first receptor in the cascade. Additional building and testing of in-series circuits will likely reveal other aspects of their design that determine their utility for therapeutic cell engineering.

The in-series circuits described here are not restricted to those that use synNotch receptors to sequentially detect dual-antigen signatures. First, in-series synNotch circuits can utilize any pair of synNotch receptors that have orthogonal synthetic transcription factors, as opposed to simply the Gal4-and LexA-based receptors described here. Second, the in-series circuits described here could be constructed using any pair of binding-triggered transcriptional switches (BTTSs) (e.g., a pair of A2 force sensor receptors, or one A2 force sensor and one TANGO receptor) with orthogonal synthetic transcription factors.

Moreover, the host cell may comprise three, four or five or more BTTSs in series. In these embodiments, the host cell may comprise, e.g., a host cell comprising: (a) a first expression cassette comprising: (i) a first promoter, and (ii) a first coding sequence encoding a first binding-triggered transcriptional switch (BTTS), wherein the first BTTS comprises a first extracellular binding domain, a transmembrane domain, and a first transcription factor, and wherein binding of the first BTTS to a first antigen on a diseased cell releases the first transcription factor into the cytoplasm of the host cell; (b) a second expression cassette, wherein transcription of the second expression cassette is induced by the released first transcription factor of (a) and comprises: (i) a second promoter, wherein the second promoter comprises a binding site for the first transcription factor, and (ii) a second coding sequence encoding a second BTTS, wherein the second BTTS comprises a second extracellular binding domain, a transmembrane domain, and a second transcription factor, wherein binding of the second BTTS to a second antigen on the diseased cell releases the second transcription factor into the cytoplasm of the host cell; and (c) a third expression cassette, wherein transcription of the second expression cassette is induced by the released second transcription factor of (b) and comprises: (i) a third promoter, wherein the third promoter comprises a binding site for the second transcription factor, and (ii) a third coding sequence encoding a third BTTS, wherein the third BTTS comprises a third extracellular binding domain, a transmembrane domain, and a third transcription factor, wherein binding of the third BTTS to a third antigen on the diseased cell releases the third transcription factor into the cytoplasm of the host cell. The third transcription factor could activate transcription of a further BTTS, for example or a therapeutic protein. This concept can be extended to four, five or more BTTSs.

One embodiment of such a circuit is illustrated in FIGS. 2A and B. In this example, the cell is a CD8 T cell, the therapeutic payload is an antigen-specific CAR, and the T cell is only activated when three antigens (in this example, EGFR, Her2 and MET) are expressed on the surface of the target cell. This circuit is “in series” because activation of a first synNotch (e.g., synNotch1, which releases a first transcription factor when it binds to EGFR) induces the expression of a second synNotch (e.g., synNotch2, which releases a second transcription factor when it binds to MET), and activation of the second synNotch induces the expression of the therapeutic payload. In this illustration, the therapeutic payload is a CAR that is activated by binding to Her2. In this example, activation of the T cell therefore requires binding to three antigens (EGFR, MET and Her2) on the target cell.

As such, in some embodiments, the host cell may comprise: (a) a first binding-triggered transcriptional switch (BTTS), wherein the first BTTS comprises a first extracellular binding domain, a transmembrane domain, and a first transcription factor, and wherein binding of the first BTTS to a first antigen on a diseased cell releases the first transcription factor into the cytoplasm of the host cell. The first BTTS may be transiently expressed in the cell by introducing an in vitro transcribed RNA encoding the first BTTS into the cell. However, in many embodiments, the first BTTS is expressed in the cell by a first expression cassette that is in the cell. In these embodiments, the first expression cassette may comprise: (i) a first promoter, and (ii) a first coding sequence encoding the first BTTS. In these embodiments, the first promoter may be constitutive in the cell, tissue-specific or inducible. If the promoter is inducible then it may have been transiently induced in order to produce the first BTTS in the cell. In addition to the first BTTS, the cell also comprises: (b) a second expression cassette, wherein transcription of the second expression cassette is induced by the released first transcription factor of (a) and comprises: (i) a second promoter, wherein the second promoter comprises a binding site for the first transcription factor, and (ii) a second coding sequence encoding a second BTTS, wherein the second BTTS comprises a second extracellular binding domain, a transmembrane domain, and a second transcription factor, wherein binding of the second BTTS to a second antigen on the diseased cell releases the second transcription factor into the cytoplasm of the host cell. In this embodiment, the host cell should also comprise: (c) a third expression cassette, wherein transcription of the third expression cassette is induced by the released second transcription factor of (b) and comprises: (i) a third promoter that comprises a binding site for the second transcription factor, and (ii) a coding sequence encoding a therapeutic protein.

As would be apparent, the first, second and third promoters, antigens, transcription factors, and extracellular binding domains are orthogonal such that binding of the first BTTS to the first antigen only activates the first BTTS (not the second BTTS or the therapeutic payload), only the second expression cassette (not the third expression cassette) is activated by the first transcription factor, binding of the second BTTS to the second antigen only activates the second BTTS (not the first BTTS or the therapeutic payload), only the third expression cassette (not the first or second expression cassettes) is activated by the second transcription factor, and the first, second and third extracellular binding domains bind to different antigens, etc.

In some embodiments, the first transcription factor is stronger than the second transcription factor, in that first transcription factor activates transcription of the second promoter more than the second transcription factor activates transcription of the third promoter. The strength of a transcription factor/promoter combination can be readily tested in vitro in the same cells using reporter constructs, e.g., GFP, before selection. In some embodiments, the first transcription factor activates transcription of the second promoter by at least 1.5×, at least 2×, at least 5× or at least 10× relative to the level that the second transcription factor activates transcription of the third promoter. As would be appreciated, the identity of the transcription factor may vary. In many embodiments, the transcription factor may have a DNA binding domain that binds to a corresponding promoter sequence and an activation domain. In many embodiments, the DNA binding domain of the first and second transcription factors may be independently selected from Gal4-, LexA-, Tet-, Lac-, dCas9-, zinc-finger- and TALE-based transcription factors. TALE- and CRISPR/dCas9-based transcription factors are described in Lebar (Methods Mol Biol. 2018 1772: 191-203), among others. The binding sites for such domains are well known or can be designed at will. The first and second transcription factors can have any suitable activation domain, e.g., VP16, VP64, Ela, Sp1, VP16, CTF, GAL4 among many others.

The expression cassettes can be in the same nucleic acid molecule (e.g., the same plasmid), in different nucleic acid molecule (in different plasmids), or two of the expression cassettes (e.g., the second and third expression cassettes) may be on the same nucleic acid molecule and the remaining expression cassette (e.g., the first expression cassette) can be on a different nucleic acid molecule. In some cases, the first and second expression cassettes are in different nucleic acid molecules, e.g., different plasmids. In some embodiments, the expression cassettes may be incorporated into the nuclear genome of the cell.

The therapeutic protein encoded by the third expression cassette can be a protein that, when expressed, is on the surface of the cell, is secreted by the cell, is in the inside of the cell (e.g., in the cytoplasm or nucleus of the cell).

For example, in some embodiments, the therapeutic protein may be a protein that, when expressed on the surface of an immune cell, activates the immune cell or inhibits activation of the immune cell when it binds to a third antigen on the diseased cell. In these embodiments, the therapeutic protein may be a chimeric antigen receptor (CAR) or a T cell receptor (TCR). In these embodiments, the third expression cassette may comprise: (i) the third promoter, and (ii) a coding sequence encoding a CAR or TCR, wherein the CAR or TCR comprises a third extracellular binding domain, a transmembrane domain, and an intracellular activation domain, wherein the CAR or TCR activates an immune cell or inhibits activation of the immune cell when it binds to the third antigen on the diseased cell. Alternatively, the therapeutic protein may be an inhibitory immune cell receptor (iICR) such as an inhibitory chimeric antigen receptor (iCAR), wherein binding of the iICR to the third antigen inhibits activation of the immune cell on which the iICR is expressed. Such iICR proteins are described in e.g., WO2017087723, Fedorov et al. (Sci. Transl. Med. 2013 5: 215ra17) and other references cited above, which are incorporated by reference for that description and examples of the same. In some embodiments such an inhibitory immunoreceptor may comprise an intracellular immunoreceptor tyrosine-based inhibition motif (ITIM), an immunoreceptor tyrosine-based switch motif (ITSM), an NpxY motif, or a YXXΦ motif. Exemplary intracellular domains for such molecules may be found in PD1, CTLA4, BTLA, CD160, KRLG-1, 2B4, Lag-3, Tim-3 and other immune checkpoints, for example. See, e.g., Odorizzi and Wherry (2012) J. Immunol. 188:2957; and Baitsch et al. (2012) PLoSOne 7: e30852.

In some embodiments, an antigen-specific therapeutic may be secreted from the cell such as an antibody. For example, the antibody may be an immune checkpoint inhibitor e.g., an antibody that binds to PD1, PD-L1, PD-L2, CTLA4, TIM3, LAG3 or another immune checkpoint. Alternatively, the secreted antigen-specific therapeutic may be a bioactive peptide such as a cytokine (e.g., Il-1ra, IL-4, IL-6, IL-10, IL-11, IL-13, or TGF-β, among many others). In some embodiments, the secreted protein may be an enzyme, e.g., a superoxide dismutase for removing reactive oxygen species, or a protease for unmasking a protease activatable antibody (e.g., a pro-body) in the vicinity of a cancer cell.

Alternatively, the therapeutic protein may be a protein that, when expressed, is internal to the cell, such as wild type or mutant SLP76, ZAP70, or Cas9 protein.

The first and second BTTSs may be independently selected from a synNotch receptor, A2 force sensor, a Modular Extracellular Sensor Architecture (MESA) and a TANGO receptor. These switches are described in greater detail below. Although other options are available, in some embodiments, the first and second BTTSs are synNotch receptors that each comprise: (i) an extracellular domain comprising the antigen binding region of an antibody; (ii) a proteolytically cleavable Notch receptor polypeptide comprising one or more proteolytic cleavage sites; and (iii) an intracellular domain comprising the first or second transcription factor, wherein binding of the extracellular domains to the first and second synNotch receptors to the first and second antigens on the diseased cell release the first and second transcription factors from the first and second BTTSs, respectively.

As will be described in greater detail below, in some embodiments the cell may be an immune cell such as a myeloid or lymphoid cell, e.g., a T lymphocyte, a B lymphocyte, a macrophage, a dendritic cell, or a natural killer cell. In these embodiments, the therapeutic protein may be a CAR, and activation of the circuit should kill the diseased cell. In other embodiments, the cell is not an immune cell. For example, the cell could be a stem cell, a brain cell or a blood cell.

The present circuit integrates the expression of two or three antigens (depending on whether the therapeutic protein is antigen-specific) on a diseased cell, e.g., a cancer cell to produce a desired outcome, e.g., death of the diseased cell.

### Methods of Treatment

As summarized above, the methods of the present disclosure find use in delivering a treatment to a subject for a disease, e.g., disease is cancer, an autoimmune disease, fibrosis, a neurodegenerative disease, diabetes, or an infectious disease. In some cases, the first and second antigens (and the third antigen, if the therapeutic protein is antigen-specific) may be antigens that are expressed by (synthesized by) a cancer cell, i.e., cancer cell-associated antigens. The cancer cell associated antigens can be antigens associated with, e.g., a breast cancer cell, a B cell lymphoma, a pancreatic cancer, a Hodgkin lymphoma cell, an ovarian cancer cell, a prostate cancer cell, a mesothelioma, a lung cancer cell (e.g., a small cell lung cancer cell), a non-Hodgkin B-cell lymphoma (B-NHL) cell, an ovarian cancer cell, a prostate cancer cell, a mesothelioma cell, a lung cancer cell (e.g., a small cell lung cancer cell), a melanoma cell, a chronic lymphocytic leukemia cell, an acute lymphocytic leukemia cell, a neuroblastoma cell, a glioma, a glioblastoma, a medulloblastoma, a colorectal cancer cell, etc.

Cancer-specific cell surface antigens are numerous and include, but are not limited to CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, and the like. Cancer-associated antigens also include, e.g., 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNTO888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R α, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin. Suitable antigen pairs or triples may be identified using the method of Orentas (Front Oncol. 2017; 7: 173) or alternatively, any of the antigen pairs/triples listed in PCT/US19/60357, could be used.

The subject methods may include introducing into a subject in need thereof, a population of the cells described above. The introduced cells may be immune cells, including e.g., myeloid cells or lymphoid cells.

In some instances, the present method may include contacting a cell with one or more nucleic acids, wherein such contacting is sufficient to introduce the nucleic acid(s) into the cell. Any convenient method of introducing nucleic acids into a cell may find use herein including but not limited viral transfection, electroporation, lipofection, bombardment, chemical transformation, use of a transducible carrier (e.g., a transducible carrier protein), and the like. Nucleic acids may be introduced into cells maintained or cultured in vitro or ex vivo. Nucleic acids may also be introduced into a cell in a living subject in vivo, e.g., through the use of one or more vectors (e.g., viral vectors) that deliver the nucleic acids into the cell without the need to isolate, culture or maintain the cells outside of the subject.

Introduced nucleic acids may be maintained within the cell or transiently present. As such, in some instance, an introduced nucleic acid may be maintained within the cell, e.g., integrated into the genome. Any convenient method of nucleic acid integration may find use in the subject methods, including but not limited to e.g., viral-based integration, transposon-based integration, homologous recombination-based integration, and the like. In some instance, an introduced nucleic acid may be transiently present, e.g., extrachromosomally present within the cell. Transiently present nucleic acids may persist, e.g., as part of any convenient transiently transfected vector.

An introduced nucleic acid encoding a circuit may be introduced in such a manner as to be operably linked to a regulatory sequence, such as a promoter, that drives the expression of one or more components of the circuit. The source of such regulatory sequences may vary and may include e.g., where the regulatory sequence is introduced with the nucleic acid, e.g., as part of an expression construct or where the regulatory sequence is present in the cell prior to introducing the nucleic acid or introduced after the nucleic acid. As described in more detail herein, useful regulatory sequence can include e.g., endogenous promoters and heterologous promoters. For example, in some instances, a nucleic acid may be introduced as part of an expression construct containing a heterologous promoter operably linked to a nucleic acid sequence. In some instances, a nucleic acid may be introduced as part of an expression construct containing a copy of a promoter that is endogenous to the cell into which the nucleic acid is introduced. In some instances, a nucleic acid may be introduced without a regulatory sequence and, upon integration into the genome of the cell, the nucleic acid may be operably linked to an endogenous regulatory sequence already present in the cell. Depending on the confirmation and/or the regulatory sequence utilized, expression of each component of the circuit from the nucleic acid may be configured to be constitutive, inducible, tissue-specific, cell-type specific, etc., including combinations thereof.

Any convenient method of delivering the circuit encoding components may find use in the subject methods. In some instances, the subject circuit may be delivered by administering to the subject a cell expressing the circuit. In some instances, the subject circuit may be delivered by administering to the subject a nucleic acid comprising one or more nucleotide sequences encoding the circuit. Administering to a subject a nucleic acid encoding the circuit may include administering to the subject a cell containing the nucleic acid where the nucleic acid may or may not yet be expressed. In some instances, administering to a subject a nucleic acid encoding the circuit may include administering to the subject a vector designed to deliver the nucleic acid to a cell.

Accordingly, in the subject methods of treatment, nucleic acids encoding a circuit or components thereof may be administered in vitro, ex vivo or in vivo. In some instances, cells may be collected from a subject and transfected with nucleic acid and the transfected cells may be administered to the subject, with or without further manipulation including but not limited to e.g., in vitro expansion. In some instances, the nucleic acid, e.g., with or without a delivery vector, may be administered directly to the subject.

Priming cells and targeted cells of a subject circuit will generally differ in at least the expression of priming antigen and targeting antigen. In some instances, priming cells and targeted cells may differ in the expression of at least one surface expressed epitope, e.g., a surfaced expressed protein, an antigen presented in the context of MHC, etc., including e.g., where the surface expressed epitope is a molecule other than the priming antigen and/or the targeting antigen. In some instances, two different targeted cells may differ in the expression of at least one surface expressed epitope, e.g., a surfaced expressed protein, an antigen presented in the context of MHC, etc.

Differential expression between two cells or two cell types may vary. For example, in some instances, a cell expresses one surface epitope not expressed by the other. In some instances, a cell expresses one surface epitope more highly than the surface epitope is expressed by the other cell. Where cells differ in the level, e.g., as compared to the presence/absence, of expression of a surface epitope the difference in level may vary but will generally be substantially different, e.g., sufficiently different to allow for practical targeting of one cell versus the other. Differences in expression between cells may range from less than one order of magnitude of expression to ten orders of magnitude of expression or more, including but not limited to e.g., 1 order of magnitude, 2 orders of magnitude, 3 orders of magnitude, 4 orders of magnitude, 5 orders of magnitude, 6 orders of magnitude, 7 orders of magnitude, 8 orders of magnitude, 9 orders of magnitude, 10 orders of magnitude, etc. In some instances, two cell types differing in level of expression of a particular epitope may be said to be “high” and “low” for the epitope, respectively, where high versus low expression may be differentiated using conventional methods known to the relevant artisan.

In some instances, the method of the present disclosure may be employed to target, treat or clear a subject for minimal residual disease (MRD) remaining after a prior therapy. Targeting, treating and/or clearance of MRD may be pursued using the instant methods whether or not the MRD is or has been determined to be refractory to the prior treatment. In some instances, a method of the present disclosure may be employed to target, treat and/or clear a subject of MRD following a determination that the MRD is refractory to a prior treatment or one or more available treatment options other than those employing the herein described circuits.

In some instances, the instant methods may be employed prophylactically for surveillance. For example, a subject in need thereof may be administered a treatment involving one or more of the herein described circuits when the subject does not have detectable disease but is at risk of developing a disease. In some instances, a prophylactic approach may be employed when a subject is at particularly high risk of developing a disease. In some instances, a prophylactic approach may be employed when a subject has been previously treated for a the disease and is at risk of reoccurrence. Essentially any combination of priming antigen and targeting antigen may be employed in prophylactic treatments, including those described herein.

The methods of treating described herein may, in some instances, be performed in a subject that has previously undergone one or more conventional treatments. For example, in the case of oncology, the methods described herein may, in some instances, be performed following a conventional cancer therapy including but not limited to e.g., conventional chemotherapy, conventional radiation therapy, conventional immunotherapy, surgery, etc. In some instances, the methods described herein may be used when a subject has not responded to or is refractory to a conventional therapy.

With respect to the disease as a whole, desired effects of the described treatments may result in a reduction in the number of diseased cells, a reduction in the size of the diseased cells, a reduction in one or more symptoms, etc.

Immune cell activation, as a result of some embodiments of the methods described herein, may be measured in a variety of ways, including but not limited to e.g., measuring the expression level of one or more markers of immune cell activation. Useful markers of immune cell activation include but are not limited to e.g., CD25, CD38, CD40L (CD154),CD69, CD71, CD95, HLA-DR, CD137 and the like. For example, in some instances, upon antigen binding by an immune cell receptor an immune cell may become activated and may express a marker of immune cell activation (e.g., CD69) at an elevated level (e.g., a level higher than a corresponding cell not bound to antigen). Levels of elevated expression of activated immune cells of the present disclosure will vary and may include an increase, such as a 1-fold or greater increase in marker expression as compared to un-activated control, including but not limited to e.g., a 1-fold increase, a 2-fold increase, a 3-fold increase, a 4-fold increase, etc.

In some instances, an immune cell modified to encode a circuit of the present disclosure, when bound to a targeted antigen, may have increased cytotoxic activity, e.g., as compared to an un-activated control cell. In some instances, activated immune cells encoding a subject circuit may show 10% or greater cell killing of antigen expressing target cells as compared to un-activated control cells. In some instances, the level of elevated cell killing of activated immune cells will vary and may range from 10% or greater, including but not limited to e.g., 20% or greater, 30% or greater, 40% or greater, 50% or greater, 60% or greater, 70% or greater, 80% or greater, 90% or greater, etc., as compared to an appropriate control.

In some instances, treatment may involve modulation, including induction, of the expression and/or secretion of a cytokine by an immune cell containing nucleic acid sequences encoding a circuit as described herein. Non-limiting examples of cytokines, the expression/secretion of which may be modulated, include but are not limited to e.g., Interleukins and related (e.g., IL-1-like, IL-1α, IL-1β, IL-1RA, IL-18, IL-2, IL-4, IL-7, IL-9, IL-13, IL-15, IL-3, IL-5, GM-CSF, IL-6-like, IL-6, IL-11, G-CSF, IL-12, LIF, OSM, IL-10-like, IL-10, IL-20, IL-14, IL-16, IL-17, etc.), Interferons (e.g., IFN-α, IFN-β, IFN-γ, etc.), TNF family (e.g., CD154, LT-β, TNF-α, TNF-β, 4-1BBL, APRIL, CD70, CD153, CD178, GITRL, LIGHT, OX40L, TALL-1, TRAIL, TWEAK, TRANCE, etc.), TGF-β family (e.g., TGF-β1, TGF-β2, TGF-β3, etc.) and the like.

In some instances, activation of an immune cell through a circuit of the present disclosure may induce an increase in cytokine expression and/or secretion relative to that of a comparable cell where the circuit is not present or otherwise inactive. The amount of the increase may vary and may range from a 10% or greater increase, including but not limited to e.g., 10% or greater, 25% or greater, 50% or greater, 75% or greater, 100% or greater, 150% or greater, 200% or greater, 250% or greater, 300% or greater, 350% or greater 400% or greater, etc.

### Conventional Treatments and Combination Therapy

As will be readily understood, the methods of treating described herein may, in some instances, be combined with one or more conventional treatments. For example, the method described herein may, in some instances, be combined with a conventional therapy including but not limited to e.g., a treatment with a drug, conventional chemotherapy, conventional radiation therapy, conventional immunotherapy, surgery, etc.

In some instances, the methods described herein may be used before or after a conventional therapy. For example, the methods described herein may be used as an adjuvant therapy, e.g., after a subject has seen improvement from a conventional therapy, or may be used when a subject has not responded to a conventional therapy. In some instances, the methods described herein may be used prior to an additional therapy, e.g., to prepare a subject for an additional therapy, e.g., a conventional therapy as described herein.

### Antigen-Specific Therapeutics

As summarized above, at least two binding triggered transcriptional switches (BTTSs) responsive to an antigen may induce the expression of an therapeutic protein responsive to the antigens. Useful antigen-specific therapeutics will vary and may include surfaced expressed and secreted antigen-specific therapeutics. For example, in some instances, an antigen-specific therapeutic used in the methods of the present disclosure may be expressed, in response to the activation of a BTTS, on the surface of an immune cell, i.e., the immune cell genetically modified to encode a priming/targeting circuit as described herein. In some instances, an antigen-specific therapeutic used in the methods of the present disclosure may be secreted, in response to the activation of a BTTS, from an immune cell, i.e., the immune cell genetically modified to encode a priming/targeting circuit as described herein.

In general, except where described otherwise, the antigen-specific therapeutic of a herein described circuit will not be expressed in the absence of the activation of the BTTS that induces its expression. Also, except where described otherwise, an antigen-specific therapeutic of a herein described circuit will not be active in the absence of the antigen to which it binds, i.e., without binding the antigen to which the antigen-specific therapeutic is specific. Binding of its respective antigen, or antigens in the case of multi- or bispecific agents, results in activation of the antigen-specific therapeutic. When expressed by, or otherwise engaged with, an immune cell and bound to antigen(s) the antigen-specific therapeutic may activate the immune cell. Activated immune cells may mediate one or more beneficial effects with respect to a diseased cell in the subject, including those described herein such as but not limited to e.g., cancer cell killing, cytokine release, and the like.

The term “antigen”, with respect to the herein described antigen-specific binding domains, is used in a broad sense to refer to essentially any specific binding partner to which the antigen-specific therapeutic binds. As such, any convenient specific binding pair, i.e., specific binding member and specific binding partner pair, may find use in the antigen-specific therapeutics of the instant methods including but not limited to e.g., antigen-antibody pairs, ligand receptor pairs, scaffold protein pairs, etc. In some instances, the specific binding member may be an antibody and its binding partner may be an antigen to which the antibody specifically binds. In some instances, the specific binding member may be a receptor and its binding partner may be a ligand to which the receptor specifically binds. In some instances, the specific binding member may be a ligand and its binding partner may be a receptor to which the ligand specifically binds.

In some instances, useful ligand-receptor specific binding pairs may include where the specific binding member is a mutein of a ligand having at least one mutation relative to the wild-type ligand, including but not limited to e.g., one or more mutations, two or more mutations, three or more mutations, four or more mutations, five or more mutations, etc. In some instances, useful muteins will have at least 90% sequence identity with the relevant wild-type amino acid sequence, including but not limited to e.g., at least 95%, at least 96%, at least 97%, at least 98%, at least 99%, etc., sequence identity with the relevant wild-type amino acid sequence. In some instances, a mutein employed in the subject polypeptide may have higher affinity for the receptor as compared to the affinity between the receptor and the wild-type ligand.

Antigen-specific therapeutics useful in the methods of the present disclosure will vary and may include but are not limited to e.g., chimeric antigen receptors (CARs), T cell receptors (TCRs), chimeric bispecific binding members, and the like.

Useful CARs include essentially any CAR useful in the treatment of cancer, including single-chain and multi-chain CARs, directed to one or more targeting antigens. A CAR used in the instant methods will generally include, at a minimum, an antigen binding domain, a transmembrane domain and an intracellular signaling domain. An employed CAR may further include one or more costimulatory domains.

Non-limiting examples of CARs that may be employed include those used in commercialized CAR T cell (CART) therapies that are directed to one or more appropriate targeting antigens or have been modified to be directed to one or more appropriate targeting antigens. In general a CAR employed herein does not target glioblastoma antigens including but not limited to e.g., EphA2, EphA3, IL13R (e.g., IL13RA1 or IL13RA2), EGFR, and ERBB2.

Useful CARs, e.g., that may be modified to be directed to an appropriate targeting antigen, or useful domains thereof, e.g., that may be employed in a CAR directed to an appropriate targeting antigens, in some instances may include those described in U.S. Pat. Nos. 9,914,909; 9,821,012; 9,815,901; 9,777,061; 9,662,405; 9,657,105; 9,629,877; 9,624,276; 9,598,489; 9,587,020; 9,574,014; 9,573,988; 9,499,629; 9,446,105; 9,394,368; 9,328,156; 9,233,125; 9,175,308 and 8,822,647; the disclosures of which are incorporated herein by reference in their entirety. In some instances, useful CARs may include or exclude heterodimeric, also referred to as dimerizable or switchable, CARs and/or include or exclude one or more of the domains thereof. Useful heterodimeric CARs and/or useful domains thereof may, in some instances, include those described in U.S. Pat. Nos. 9,587,020 and 9,821,012 as well as U.S. Pub. Nos. US20170081411A1, US20160311901A1, US20160311907A1, US20150266973A1 and PCT Pub. Nos. WO2014127261A1, WO2015142661A1, WO2015090229A1 and WO2015017214A1; the disclosures of which are incorporated herein by reference in their entirety.

As summarized above, in some instances, the antigen binding domain of a CAR, such but not limited to e.g., those described in any one of the documents referenced above, may be substituted or amended with an alternative or additional antigen binding domain directed to a different antigen, such as but not limited to one or more of the antigens described herein, for use in the herein described methods. In such instances, the intracellular portions (i.e., the intracellular signaling domain or the one or more co-stimulatory domains) of the antigen-domain-substituted CAR may or may not be modified.

Useful CARs and/or useful domains thereof may, in some instances, include those that have been or are currently being investigated in one or more clinical trials, including but not limited to the CARs directed to the following antigens (listed with an exemplary corresponding clinical trial number, further information pertaining to which may be retrieved by visiting www(dot)clinicaltrials(dot)gov): AFP, e.g., in NCT03349255; BCMA, e.g., in NCT03288493; CD10, e.g., in NCT03291444; CD117, e.g., in NCT03291444; CD123, e.g., in NCT03114670; CD133, e.g., in NCT02541370; CD138 , e.g., in NCT01886976; CD171, e.g., in NCT02311621; CD19, e.g., in NCT02813252; CD20, e.g., in NCT03277729; CD22, e.g., in NCT03244306; CD30, e.g., in NCT02917083; CD33, e.g., in NCT03126864; CD34, e.g., in NCT03291444; CD38, e.g., in NCT03291444; CD5, e.g., in NCT03081910; CD56, e.g., in NCT03291444; CD7, e.g., in NCT02742727; CD70, e.g., in NCT02830724; CD80, e.g., in NCT03356808; CD86, e.g., in NCT03356808; CEA, e.g., in NCT02850536; CLD18, e.g., in NCT03159819; CLL-1, e.g., in NCT03312205; cMet, e.g., in NCT01837602; EGFR, e.g., in NCT03182816; EGFRvIII, e.g., in NCT02664363; EpCAM, e.g., in NCT03013712; EphA2, e.g., in NCT02575261; GD-2, e.g., in NCT01822652; Glypican 3, e.g., in NCT02905188; GPC3, e.g., in NCT02723942; HER-2, e.g., in NCT02547961; kappa immunoglobulin, e.g., in NCT00881920; LeY, e.g., in NCT02958384; LMP1, e.g., in NCT02980315; mesothelin, e.g., in NCT02930993; MG7, e.g., in NCT02862704; MUC1, e.g., in NCT02587689; NKG2D-ligands, e.g., in NCT02203825; PD-L1, e.g., in NCT03330834; PSCA, e.g., in NCT02744287; PSMA, e.g., in NCT03356795; ROR1, e.g., in NCT02706392; ROR1R, e.g., in NCT02194374; TACI, e.g., in NCT03287804; and VEGFR2, e.g., in NCT01218867.

Useful TCRs include essentially any TCR useful in the treatment of cancer, including single-chain and multi-chain TCRs, directed to a targeting antigen. A TCR used in the instant methods will generally include, at a minimum, an antigen binding domain and a modified or unmodified TCR chain, or portion thereof, including but not limited to e.g., a modified or unmodified α-chain, a modified or unmodified β-chain, etc. An employed TCR may further include one or more costimulatory domains. In some instances, a TCR employed herein will include an alpha chain and a beta chain and recognize antigen when presented by a major histocompatibility complex.

Essentially any TCR can be induced by a BTTS using a method of the present disclosure including e.g., TCRs that are specific for any of a variety of epitopes, including, e.g., an epitope expressed on the surface of a cancer cell, a peptide-MHC complex on the surface of cancer cell, and the like. In some cases, the TCR is an engineered TCR.

Non-limiting examples of engineered TCRs, including those having immune cell activation function and that may be modified to include an antigen-binding domain specific for a suitable targeting antigen, useful in the methods described herein include, e.g., antigen-specific TCRs, Monoclonal TCRs (MTCRs), Single chain MTCRs, High Affinity CDR2 Mutant TCRs, CD1-binding MTCRs, High Affinity NY-ESO TCRs, VYG HLA-A24 Telomerase TCRs, including e.g., those described in PCT Pub Nos. WO 2003/020763, WO 2004/033685, WO 2004/044004, WO 2005/114215, WO 2006/000830, WO 2008/038002, WO 2008/039818, WO 2004/074322, WO 2005/113595, WO 2006/125962; Strommes et al. Immunol Rev. 2014; 257(1):145-64; Schmitt et al. Blood. 2013; 122(3):348-56; Chapuls et al. Sci Transl Med. 2013; 5(174):174ra27; Thaxton et al. Hum Vaccin Immunother. 2014; 10(11):3313-21 (PMID:25483644); Gschweng et al. Immunol Rev. 2014; 257(1):237-49 (PMID:24329801); Hinrichs et al. Immunol Rev. 2014; 257(1):56-71 (PMID:24329789); Zoete et al. Front Immunol. 2013; 4:268 (PMID:24062738); Man et al. Clin Exp Immunol. 2012; 167(2):216-25 (PMID:22235997); Zhang et al. Adv Drug Deliv Rev. 2012; 64(8):756-62 (PMID:22178904); Chhabra et al. Scientific World Journal. 2011; 11:121-9 (PMID:21218269); Boulter et al. Clin Exp Immunol. 2005; 142(3):454-60 (PMID:16297157); Sami et al. Protein Eng Des Sel. 2007; 20(8):397-403; Boulter et al. Protein Eng. 2003; 16(9):707-11; Ashfield et al. IDrugs. 2006; 9(8):554-9; Li et al. Nat Biotechnol. 2005; 23(3):349-54; Dunn et al. Protein Sci. 2006; 15(4):710-21; Liddy et al. Mol Biotechnol. 2010; 45(2); Liddy et al. Nat Med. 2012; 18(6):980-7; Oates, et al. Oncoimmunology. 2013; 2(2):e22891; McCormack, et al. Cancer Immunol Immunother. 2013 April; 62(4):773-85; Bossi et al. Cancer Immunol Immunother. 2014; 63(5):437-48 and Oates, et al. Mol Immunol. 2015 October; 67(2 Pt A):67-74; the disclosures of which are incorporated herein by reference in their entirety.

Useful TCRs include those having wild-type affinity for their respective antigen as well as those having enhanced affinity for their respective antigen. TCRs having enhanced affinity for their respective antigen may be referred to as “affinity enhanced” or “enhanced affinity” TCRs. The affinity of a TCR may be enhanced by any convenient means, including but not limited to binding-site engineering (i.e., rational design), screening (e.g., TCR display), or the like. Non-limiting examples of affinity enhanced TCRs and methods of generating enhanced affinity TCRs include but are not limited to e.g., those described in PCT Pub. Nos. 20150118208, 2013256159, 20160083449; 20140349855, 20100113300, 20140371085, 20060127377, 20080292549, 20160280756, 20140065111, 20130058908, 20110038842, 20110014169, 2003276403 and the like; the disclosures of which are incorporated herein by reference in their entirety. Further engineered TCRs, modified to be directed to an appropriate targeting antigen, that may be expressed in response to release of an intracellular domain of a BTTS of the present disclosure include e.g., those described in PCT Application No. US2017/048040; the disclosure of which is incorporated herein by reference in its entirety.

Useful TCRs, which may be modified to be directed to an appropriate targeting antigen, may, in some instances, also include those described in U.S. Pat. Nos. 9,889,161; 9,889,160; 9,868,765; 9,862,755; 9,717,758; 9,676,867; 9,409,969; 9,115,372; 8,951,510; 8,906,383; 8,889,141; 8,722,048; 8,697,854; 8,603,810; 8,383,401; 8,361,794; 8,283,446; 8,143,376; 8,003,770; 7,998,926; 7,666,604; 7,456,263; 7,446,191; 7,446,179; 7,329,731; 7,265,209; and 6,770,749; the disclosures of which are incorporated herein by reference in their entirety.

As described above, in some instances, the antigen binding domain of a TCR, such as but not limited to e.g., those described or referenced above, may be substituted or amended with an alternative or additional antigen binding domain directed to a different antigen, such as but not limited to one or more of the antigens described herein, for use in the herein described methods. In such instances, the other portions (i.e., the transmembrane domain, any intracellular signaling domains, etc.) of the antigen-domain-substituted TCR may or may not be modified.

As summarized above, in some instances, useful antigen-specific therapeutics may include those that, upon induction by an activated BTTS, are expressed and secreted from the producing cell, including e.g., where the secreting cell is an immune cell. For example, upon binding of a BTTS expressed by an immune cell, the BTTS may induce expression and secretion of an encoded antigen-specific therapeutic specific for a targeting antigen. The secreted antigen-specific therapeutic may target a target antigen expressing cancer cell in trans, thereby mediating killing of the target cell. As described herein, in some instances, a secreted antigen-specific therapeutic may increase the zone of targeting or the zone of killing of a subject circuit as compared to a similar circuit encoding a non-secreted (e.g., membrane expressed) antigen-specific therapeutic.

Useful secreted antigen-specific therapeutics will vary and in some instances may include but are not limited to e.g., chimeric bispecific binding members. In some instances, useful chimeric bispecific binding members may include those that target a protein expressed on the surface of an immune cell, including but not limited to e.g., a component of the T cell receptor (TCR), e.g., one or more T cell co-receptors. Chimeric bispecific binding members that bind to a component of the TCR may be referred to herein as a TCR-targeted bispecific binding agent. Chimeric bispecific binding members useful in the instant methods will generally be specific for a targeting antigen and may, in some instances, be specific for a targeting antigen and a protein expressed on the surface of an immune cell (e.g.,. a component of a TCR such as e.g., a CD3 co-receptor).

In some instances, useful chimeric bispecific binding members may include a bispecific T cell engager (BiTE). A BiTE is generally made by fusing a specific binding member (e.g., a scFv) that binds an immune cell antigen to a specific binding member (e.g., a scFv) that binds a cancer antigen (e.g., a tumor associated antigen, a tumor specific antigen, etc.). For example, an exemplary BiTE includes an anti-CD3 scFv fused to an anti-tumor associated antigen (e.g., EpCAM, CD19, etc.) scFv via a short peptide linker (e.g., a five amino acid linker).

As summarized above, in some instances, the antigen binding domain of a chimeric bispecific binding member, such as but not limited to e.g., those described or referenced above, may be substituted or amended with an alternative or additional antigen binding domain directed to a different antigen, such as but not limited to one or more of the antigens described herein, for use in the herein described methods. In such instances, the other portions (i.e., linker domain, any immune cell targeting domains, etc.) of the antigen-domain-substituted chimeric bispecific binding member may or may not be modified.

In some instances, a payload induced by binding of a BTTS to its respective priming antigen in a herein described method may include a secreted bio-orthogonal adapter molecule. Such bio-orthogonal adapter molecules may, in some instances, be configured to target and bind a targeting antigen and also bind or be bound by a heterologous polypeptide expressed by an immune cell.

For example, in some instances, a subject circuit employed in the herein described methods may encode, within an immune cell: a BTTS responsive to a priming antigen; a bio-orthogonal adapter molecule specific for a targeting antigen; and a therapeutic, or portion thereof, which binds the bio-orthogonal adapter molecule. In such a circuit, expression and secretion of the bio-orthogonal adapter molecule is induced upon binding of the BTTS to the priming antigen. Then, in the presence of both (1) a cancer cell expressing the targeting antigen and (2) the therapeutic that binds the bio-orthogonal adapter molecule, the therapeutic binds the bio-orthogonal adapter molecule which then binds the targeting antigen, thereby activating the therapeutic. The activated therapeutic may then mediate a therapeutic effect (e.g., a cytotoxic effect) on the diseased cell expressing the targeting antigen, including where the targeting antigen is expressed in trans with respect to the priming antigen. As described herein, in some instances, a secreted bio-orthogonal adapter molecule may increase the zone of targeting or the zone of killing of a subject circuit as compared to a similar circuit encoding a non-secreted (e.g., membrane expressed) antigen-specific therapeutic.

Bio-orthogonal adapter molecules may be employed in various contexts within the herein described methods. For example, in some instances, a bio-orthogonal adapter molecule may be employed that includes a diffusible antigen binding portion of an antigen-specific therapeutic, such as e.g., a diffusible antigen binding portion of a CAR, a diffusible antigen binding portion of a TCR, or the like. In some instances, such diffusible antigen binding portion of antigen-specific therapeutics may be referred to a “diffusible head”, including e.g., a “diffusible CAR head”, a “diffusible TCR head”, and the like.

In some instances, the therapeutic may bind directly to the bio-orthogonal adapter molecule. Strategies for direct binding of the therapeutic to the bio-orthogonal adapter molecule may vary. For example, in some instances, the therapeutic may include a binding domain (e.g., such as an orthogonal antibody or fragment thereof) that binds a binding moiety (e.g., an orthogonal epitope to which an antibody may be directed) covalently attached to the bio-orthogonal adapter. As a non-limiting example, a therapeutic may include a binding domain to a non-naturally occurring epitope, e.g., an anti-fluorescein antibody or a fragment thereof, and the bio-orthogonal adapter molecule may include the epitope, e.g., a fluorescein, covalently attached thereto. In some instances, the configuration of the bio-orthogonal adapter molecule and therapeutic interaction may be reversed as compared to that previously described, including e.g., where the therapeutic includes a covalently attached epitope and the bio-orthogonal adapter molecule includes a binding domain to the epitope. Useful epitopes will vary and may include but are not limited to e.g., small molecule-based epitopes, peptide-based epitopes (e.g., peptide neo-epitopes), oligonucleotide-based epitopes, and the like. The epitope-binding domains will vary correspondingly and may include but are not limited to e.g., small molecule binding domains, peptide binding domains, oligonucleotide binding domains, and the like.

Non-limiting examples of useful bio-orthogonal adapter molecules, and the domains that bind thereto, include but are not limited to e.g., the peptide neo-epitopes and the antibody binding domains that bind thereto as used in switchable CAR (sCAR) T cells, including but not limited to e.g., those described in Rodgers et al. Proc Natl Acad Sci USA. (2016) 113(4):E459-68 and Cao et al., Angew Chem Int Ed Engl. 2016 Jun. 20; 55(26):7520-4 as well as PCT Pub. No. WO2016168773; the disclosures of which are incorporated herein by reference in their entirety.

In some instances, the therapeutic may bind indirectly to the bio-orthogonal adapter molecule, including e.g., where binding is mediated by a diffusible dimerizing agent. Non-limiting examples of suitable dimerizing agents, and the dimerizing domains that bind thereto, include protein dimerizers.

Protein dimerizers generally include polypeptide pairs that dimerize, e.g., in the presence of or when exposed to a dimerizing agent. The dimerizing polypeptide pairs of a protein dimerizer may homo-dimerize or hetero-dimerize (i.e., the dimerizing polypeptide pairs may include two of the same polypeptide that form a homodimer or two different polypeptides that form a heterodimer). Non-limiting pairs of protein dimerizers (with the relevant dimerizing agent in parentheses) include but are not limited to e.g., FK506 binding protein (FKBP) and FKBP (rapamycin); FKBP and calcineurin catalytic subunit A (CnA) (rapamycin); FKBP and cyclophilin (rapamycin); FKBP and FKBP-rapamycin associated protein (FRB) (rapamycin); gyrase B (GyrB) and GyrB (coumermycin); dihydrofolate reductase (DHFR) and DHFR (methotrexate); DmrB and DmrB (AP20187); PYL and ABI (abscisic acid); Cry2 and CIB1 (blue light); GAI and GID1 (gibberellin); and the like. Further description, including the amino acid sequences, of such protein dimerizers is provided in U.S. Patent Application Publication No. US 2015-0368342 A1; the disclosure of which is incorporated herein by reference in its entirety.

Useful protein dimerizers also include those nuclear hormone receptor derived protein dimerizers that dimerize in the presence of a dimerizing agent described in PCT Pub. No. WO 2017/120546 and U.S. Patent Pub. No. US 2017/0306303 A1; the disclosures of which are incorporated by reference herein in their entirety, and the like. Such nuclear hormone receptor derived dimerizers will generally include a first member of the dimerization pair that is a co-regulator of a nuclear hormone receptor and a second member of the dimerization pair comprises an LBD of the nuclear hormone receptor.

Where a bio-orthogonal adapter molecule is employed in a subject circuit, the expression of the therapeutic, which binds the bio-orthogonal adapter molecule to mediate targeting antigen recognition, may or may not be controlled by the circuit. Put another way, the expression of the therapeutic may or may not be tied to the activation of the BTTS (e.g., the binding of the BTTS to priming antigen or another antigen) of the circuit. In some instances, the circuit may be configured such that binding of a BTTS to its antigen induces expression of a therapeutic which binds a bio-orthogonal adapter molecule. In some instances, the BTTS that induces expression of the therapeutic is the same BTTS that induces expression of the bio-orthogonal adapter molecule. In some instance, the therapeutic is induced by a BTTS that is different (i.e., separate) from the BTTS that induces expression of the bio-orthogonal adapter molecule.

In some instances, expression of a therapeutic which binds a bio-orthogonal adapter molecule may not be induced by a BTTS. For example, in some instances, rather than being induced by a BTTS, such a therapeutic is expressed under the control of a separate regulatory element or sequence, including but not limited to e.g., where the expression of the therapeutic is constitutive, inducible, conditional, tissue specific, cell type specific, or the like. In some instances, for example, independent expression (e.g., constitutive expression, inducible expression, etc.) of the therapeutic by introduced immune cells allows for a diffusible bio-orthogonal adapter molecule to mediate the activation of the therapeutic in immune cells that are distant from the site of priming.

In some instances, expression of a bio-orthogonal adapter molecule, bound by a therapeutic, may not be induced by a BTTS, including where the corresponding therapeutic is induced by a BTTS. For example, in some instances, rather than being induced by a BTTS, such a bio-orthogonal adapter molecule is expressed under the control of a separate regulatory element or sequence, including but not limited to e.g., where the expression of the bio-orthogonal adapter molecule is constitutive, inducible, conditional, tissue specific, cell type specific, or the like. In some instances, the bio-orthogonal adapter molecule may be externally provided.

In some instances, an antigen-specific therapeutic may have an extracellular domain that includes a first member of a specific binding pair that binds a second member of the specific binding pair, wherein the extracellular domain does not include any additional first or second member of a second specific binding pair. For example, in some instances, an antigen-specific therapeutic may have an extracellular domain that includes a first antigen-binding domain that binds an antigen, wherein the extracellular domain does not include any additional antigen-binding domains and does not bind any other antigens. A subject antigen-specific therapeutic may, in some instances, include only a single extracellular domain. Accordingly, an employed antigen-specific therapeutic may be specific for a single antigen and only specific for the single antigen. Such, antigen-specific therapeutics may be referred to as a “single antigen antigen-specific therapeutic”.

In some instances, an antigen-specific therapeutic may have an extracellular domain that includes the first or second members of two or more specific binding pairs. For example, in some instances, an antigen-specific therapeutic may have an extracellular domain that includes a first antigen-binding domain and a second antigen-binding domain that are different such that the extracellular domain is specific for two different antigens. In some instances, an antigen-specific therapeutic may have two or more extracellular domains that each includes the first or second members of two different specific binding pairs. For example, in some instances, an antigen-specific therapeutic may have a first extracellular domain that includes a first antigen-binding domain and a second extracellular domain that includes a second antigen-binding domain where the two different antigen binding domains are each specific for a different antigen. As such, the antigen-specific therapeutic may be specific for two different antigens.

An antigen-specific therapeutic specific for two or more different antigens, containing either two extracellular domains or one extracellular domain specific for two different antigens, may be configured such that the binding of either antigen to the antigen-specific therapeutic is sufficient to active the antigen-specific therapeutic. Such an antigen-specific therapeutic, capable of being activated by any of two or more antigens, may find use in the described circuits as a component of a logic gate containing OR functionality. In some instances, an antigen-specific therapeutic specific for two different antigens may be referred to as a “two-headed antigen-specific therapeutic”. Antigen-specific therapeutics specific for multiple antigens will not be limited to only two antigens and may, e.g., be specific for and/or activated by more than two antigens, including e.g., three or more, four or more, five or more, etc.

An example of an antigen-specific therapeutic specific for two or more different antigens is a tandem CAR (also referred to as “tan CAR” or “tanCAR”). A “tandem CAR” is a bispecific CAR that includes two or more non-identical antigen recognition domains. Non-limiting examples of tandem CARs include those described in U.S. Pat. Nos. 9,447,194; 10,155,038; 10,189,903; and 10,239,948; U.S. Patent Application Pub. No. 20130280220 and PCT Application Pub. No. WO/2013/123061; the disclosures of which are incorporated herein by reference in their entirety. Tandem CARs may be configured to bind a variety of different antigens, including but not limited to e.g., two or more or the antigens described herein and/or two or more of the antigens described in U.S. Pat. Nos. 9,447,194; 10,155,038; 10,189,903; and 10,239,948; U.S. Patent Application Pub. No. 20130280220 and PCT Application Pub. No. WO/2013/123061.

Binding Triggered Transcriptional Switches (BTTS)

The method of the present disclosure may include the use of circuits employing a BTTS to induce expression of an encoded antigen-specific therapeutic. As used herein, a “binding-triggered transcriptional switch” or BTTS generally refers to a synthetic modular polypeptide or system of interacting polypeptides having an extracellular domain that includes a first member of a specific binding pair, a binding-transducer and an intracellular domain. Upon binding of the second member of the specific binding pair to the BTTS the binding signal is transduced to the intracellular domain such that the intracellular domain becomes activated and performs some function within the cell that it does not perform in the absence of the binding signal. Binding triggered transcriptional switches are described in e.g., PCT Pub. No. WO 2016/138034 as well as U.S. Pat. Nos. 9,670,281 and 9,834,608; the disclosures of which are incorporated herein by reference in their entirety.

The specific binding member of the extracellular domain generally determines the specificity of the BTTS. In some instances, a BTTS may be referred according to its specificity as determined based on its specific binding member. For example, a specific binding member having binding partner “X” may be referred to as an X-BTTS or an anti-X BTTS.

Any convenient specific binding pair, i.e., specific binding member and specific binding partner pair, may find use in the BTTS of the instant methods including but not limited to e.g., antigen-antibody pairs, ligand receptor pairs, scaffold protein pairs, etc. In some instances, the specific binding member may be an antibody and its binding partner may be an antigen to which the antibody specifically binds. In some instances, the specific binding member may be a receptor and its binding partner may be a ligand to which the receptor specifically binds. In some instances, the specific binding member may be a scaffold protein and its binding partner may be a protein to which the scaffold protein specifically binds. Useful specific binding pairs include those specific for priming antigen and/or one or more targeting/killing antigens, including those described herein.

In some cases, the specific binding member is an antibody. The antibody can be any antigen-binding antibody-based polypeptide, a wide variety of which are known in the art. In some instances, the specific binding member is or includes a monoclonal antibody, a single chain Fv (scFv), a Fab, etc. Other antibody based recognition domains (cAb VHH (camelid antibody variable domains) and humanized versions, IgNAR VH (shark antibody variable domains) and humanized versions, sdAb VH (single domain antibody variable domains) and “camelized” antibody variable domains are suitable for use. In some instances, T-cell receptor (TCR) based recognition domains such as single chain TCR (scTv, single chain two-domain TCR containing VαVβ) are also suitable for use.

Where the specific binding member of a BTTS is an antibody-based binding member, the BTTS can be activated in the presence of a binding partner to the antibody-based binding member, including e.g., an antigen specifically bound by the antibody-based binding member. In some instances, antibody-based binding member may be defined, as is commonly done in the relevant art, based on the antigen bound by the antibody-based binding member, including e.g., where the antibody-based binding member is described as an “anti-” antigen antibody, e.g., an anti-priming antigen antibody (e.g., an anti-IL13RA2 antibody, anti-IL13RA1 antibody, anti-Neuroligin antibody, anti-NRXN1 antibody, anti-PTPRZ1 antibody, anti-NRCAM antibody, anti-CDH10 antibody, anti-PCDHGC5 antibody, anti-CD70 antibody anti-CSPG5 antibody, anti-BCAN antibody, anti-GRM3 antibody, anti-CRB1 antibody, anti-GAP43 antibody, anti-ATP1B2 antibody, anti-PTPRZ1-MET fusion antibody, etc.). Accordingly, antibody-based binding members suitable for inclusion in a BTTS or an antigen-specific therapeutic of the present methods can have a variety of antigen-binding specificities.

The components of BTTSs, employed in the described methods, and the arrangement of the components of the switch relative to one another will vary depending on many factors including but not limited to e.g., the desired binding trigger, the activity of the intracellular domain, the overall function of the BTTS, the broader arrangement of a molecular circuit comprising the BTTS, etc. The first binding member may include but is not limited to e.g., those agents that bind an antigen described herein. The intracellular domain may include but is not limited e.g., those intracellular domains that activate or repress transcription at a regulatory sequence, e.g., to induce or inhibit expression of a downstream component of a particular circuit.

The binding transducer of BTTSs will also vary depending on the desired method of transduction of the binding signal. Generally, binding transducers may include those polypeptides and/or domains of polypeptides that transduce an extracellular signal to intracellular signaling e.g., as performed by the receptors of various signal transduction pathways. Transduction of a binding signal may be achieved through various mechanisms including but not limited to e.g., binding-induced proteolytic cleavage, binding-induced phosphorylation, binding-induced conformational change, etc. In some instances, a binding-transducer may contain a ligand-inducible proteolytic cleavage site such that upon binding the binding-signal is transduced by cleavage of the BTTS, e.g., to liberate an intracellular domain. For example, in some instances, a BTTS may include a Notch derived cleavable binding transducer, such as, e.g., a chimeric notch receptor polypeptide as described herein.

In other instances, the binding signal may be transduced in the absence of inducible proteolytic cleavage. Any signal transduction component or components of a signaling transduction pathway may find use in a BTTS whether or not proteolytic cleavage is necessary for signal propagation. For example, in some instances, a phosphorylation-based binding transducer, including but not limited to e.g., one or more signal transduction components of the Jak-Stat pathway, may find use in a non-proteolytic BTTS.

For simplicity, BTTSs, including but not limited to chimeric notch receptor polypeptides, are described primarily as single polypeptide chains. However, BTTSs, including chimeric notch receptor polypeptides, may be divided or split across two or more separate polypeptide chains where the joining of the two or more polypeptide chains to form a functional BTTS, e.g., a chimeric notch receptor polypeptide, may be constitutive or conditionally controlled. For example, constitutive joining of two portions of a split BTTS may be achieved by inserting a constitutive heterodimerization domain between the first and second portions of the split polypeptide such that upon heterodimerization the split portions are functionally joined.

Useful BTTSs that may be employed in the subject methods include, but are not limited to modular extracellular sensor architecture (MESA) polypeptides. A MESA polypeptide comprises: a) a ligand binding domain; b) a transmembrane domain; c) a protease cleavage site; and d) a functional domain. The functional domain can be a transcription regulator (e.g., a transcription activator, a transcription repressor). In some cases, a MESA receptor comprises two polypeptide chains. In some cases, a MESA receptor comprises a single polypeptide chain. Non-limiting examples of MESA polypeptides are described in, e.g., U.S. Patent Publication No. 2014/0234851; the disclosure of which is incorporated herein by reference in its entirety.

Useful BTTSs that may be employed in the subject methods include, but are not limited to polypeptides employed in the TANGO assay. The subject TANGO assay employs a TANGO polypeptide that is a heterodimer in which a first polypeptide comprises a tobacco etch virus (Tev) protease and a second polypeptide comprises a Tev proteolytic cleavage site (PCS) fused to a transcription factor. When the two polypeptides are in proximity to one another, which proximity is mediated by a native protein-protein interaction, Tev cleaves the PCS to release the transcription factor. Non-limiting examples of TANGO polypeptides are described in, e.g., Barnea et al. (Proc Natl Acad Sci USA. 2008 Jan. 8; 105(1):64-9); the disclosure of which is incorporated herein by reference in its entirety.

Useful BTTSs that may be employed in the subject methods include, but are not limited to von Willebrand Factor (vWF) cleavage domain-based BTTSs, such as but not limited to e.g., those containing a unmodified or modified vWF A2 domain. A subject vWF cleavage domain-based BTTS will generally include: an extracellular domain comprising a first member of a binding pair; a von Willebrand Factor (vWF) cleavage domain comprising a proteolytic cleavage site; a cleavable transmembrane domain and an intracellular domain. Non-limiting examples of vWF cleavage domains and vWF cleavage domain-based BTTSs are described in Langridge & Struhl (Cell (2017) 171(6):1383-1396); the disclosure of which is incorporated herein by reference in its entirety.

Useful BTTSs that may be employed in the subject methods include, but are not limited to chimeric Notch receptor polypeptides, such as but not limited to e.g., synNotch polypeptides, non-limiting examples of which are described in PCT Pub. No. WO 2016/138034, U.S. Pat. Nos. 9,670,281, 9,834,608, Roybal et al. Cell (2016) 167(2):419-432, Roybal et al. Cell (2016) 164(4):770-9, and Morsut et al. Cell (2016) 164(4):780-91; the disclosures of which are incorporated herein by reference in their entirety.

SynNotch polypeptides are generally proteolytically cleavable chimeric polypeptides that generally include: a) an extracellular domain comprising a specific binding member; b) a proteolytically cleavable Notch receptor polypeptide comprising one or more proteolytic cleavage sites; and c) an intracellular domain. Binding of the specific binding member by its binding partner generally induces cleavage of the synNotch at the one or more proteolytic cleavage sites, thereby releasing the intracellular domain. In some instances, the instant methods may include where release of the intracellular domain triggers (i.e., induces) the production of an encoded payload, the encoding nucleic acid sequence of which is contained within the cell. Depending on the particular context, the produced payload is then generally expressed on the cell surface or secreted. SynNotch polypeptides generally include at least one sequence that is heterologous to the Notch receptor polypeptide (i.e., is not derived from a Notch receptor), including e.g., where the extracellular domain is heterologous, where the intracellular domain is heterologous, where both the extracellular domain and the intracellular domain are heterologous to the Notch receptor, etc.

Useful synNotch BTTSs will vary in the domains employed and the architecture of such domains. SynNotch polypeptides will generally include a Notch receptor polypeptide that includes one or more ligand-inducible proteolytic cleavage sites. The length of Notch receptor polypeptides will vary and may range in length from about 50 amino acids or less to about 1000 amino acids or more.

In some cases, the Notch receptor polypeptide present in a synNotch polypeptide has a length of from 50 amino acids (aa) to 1000 aa, e.g., from 50 aa to 75 aa, from 75 aa to 100 aa, from 100 aa to 150 aa, from 150 aa to 200 aa, from 200 aa to 250 aa, from 250 a to 300 aa, from 300 aa to 350 aa, from 350 aa to 400 aa, from 400 aa to 450 aa, from 450 aa to 500 aa, from 500 aa to 550 aa, from 550 aa to 600 aa, from 600 aa to 650 aa, from 650 aa to 700 aa, from 700 aa to 750 aa, from 750 aa to 800 aa, from 800 aa to 850 aa, from 850 aa to 900 aa, from 900 aa to 950 aa, or from 950 aa to 1000 aa. In some cases, the Notch receptor polypeptide present in a synNotch polypeptide has a length of from 300 aa to 400 aa, from 300 aa to 350 aa, from 300 aa to 325 aa, from 350 aa to 400 aa, from 750 aa to 850 aa, from 50 aa to 75 aa. In some cases, the Notch receptor polypeptide has a length of from 310 aa to 320 aa, e.g., 310 aa, 311 aa, 312 aa, 313 aa, 314 aa, 315 aa, 316 aa, 317 aa, 318 aa, 319 aa, or 320 aa. In some cases, the Notch receptor polypeptide has a length of 315 aa. In some cases, the Notch receptor polypeptide has a length of from 360 aa to 370 aa, e.g., 360 aa, 361 aa, 362 aa, 363 aa 364 aa, 365 aa, 366 aa, 367 aa, 368 aa, 369 aa, or 370 aa. In some cases, the Notch receptor polypeptide has a length of 367 aa.

In some cases, a Notch receptor polypeptide comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence of a Notch receptor. In some instances, the Notch regulatory region of a Notch receptor polypeptide is a mammalian Notch regulatory region, including but not limited to e.g., a mouse Notch (e.g., mouse Notch1, mouse Notch2, mouse Notch3 or mouse Notch4) regulatory region, a rat Notch regulatory region (e.g., rat Notch1, rat Notch2 or rat Notch3), a human Notch regulatory region (e.g., human Notch1, human Notch2, human Notch3 or human Notch4), and the like or a Notch regulatory region derived from a mammalian Notch regulatory region and having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence of a mammalian Notch regulatory region of a mammalian Notch receptor amino acid sequence.

Subject Notch regulatory regions may include or exclude various components (e.g., domains, cleavage sites, etc.) thereof. Examples of such components of Notch regulatory regions that may be present or absent in whole or in part, as appropriate, include e.g., one or more EGF-like repeat domains, one or more Lin12/Notch repeat domains, one or more heterodimerization domains (e.g., HD-N or HD-C), a transmembrane domain, one or more proteolytic cleavage sites (e.g., a furin-like protease site (e.g., an S1 site), an ADAM-family protease site (e.g., an S2 site) and/or a gamma-secretase protease site (e.g., an S3 site)), and the like. Notch receptor polypeptides may, in some instances, exclude all or a portion of one or more Notch extracellular domains, including e.g., Notch-ligand binding domains such as Delta-binding domains. Notch receptor polypeptides may, in some instances, include one or more non-functional versions of one or more Notch extracellular domains, including e.g., Notch-ligand binding domains such as Delta-binding domains. Notch receptor polypeptides may, in some instances, exclude all or a portion of one or more Notch intracellular domains, including e.g., Notch Rbp-associated molecule domains (i.e., RAM domains), Notch Ankyrin repeat domains, Notch transactivation domains, Notch PEST domains, and the like. Notch receptor polypeptides may, in some instances, include one or more non-functional versions of one or more Notch intracellular domains, including e.g., non-functional Notch Rbp-associated molecule domains (i.e., RAM domains), non-functional Notch Ankyrin repeat domains, non-functional Notch transactivation domains, non-functional Notch PEST domains, and the like.

Non-limiting examples of particular synNotch BTTSs, the domains thereof, and suitable domain arrangements are described in PCT Pub. Nos. WO 2016/138034, WO 2017/193059, WO 2018/039247 and U.S. Pat. Nos. 9,670,281 and 9,834,608; the disclosures of which are incorporated herein by reference in their entirety.

Domains of a useful BTTS, e.g., the extracellular domain, the binding-transducer domain, the intracellular domain, etc., may be joined directly, i.e., with no intervening amino acid residues or may include a peptide linker that joins two domains. Peptide linkers may be synthetic or naturally derived including e.g., a fragment of a naturally occurring polypeptide.

A peptide linker can vary in length of from about 3 amino acids (aa) or less to about 200 aa or more, including but not limited to e.g., from 3 aa to 10 aa, from 5 aa to 15 aa, from 10 aa to 25 aa, from 25 aa to 50 aa, from 50 aa to 75 aa, from 75 aa to 100 aa, from 100 aa to 125 aa, from 125 aa to 150 aa, from 150 aa to 175 aa, or from 175 aa to 200 aa. A peptide linker can have a length of from 3 aa to 30 aa, e.g., 3, 4, 5, 6, 7, 8, 9, 10, 11, 12, 13, 14, 15, 16, 17, 18, 19, 20, 21, 22, 23, 24, 25, 26, 27, 28, 29, or 30 aa. A peptide linker can have a length of from 5 aa to 50 aa, e.g., from 5 aa to 40 aa, from 5 aa to 35 aa, from 5 aa to 30 aa, from 5 aa to 25 aa, from 5 aa to 20 aa, from 5 aa to 15 aa or from 5 aa to 10 aa.

In some instances, a BTTS may have an extracellular domain that includes a first member of a specific binding pair that binds a second member of the specific binding pair, wherein the extracellular domain does not include any additional first or second member of a second specific binding pair. For example, in some instances, a BTTS may have an extracellular domain that includes a first antigen-binding domain that binds an antigen, wherein the extracellular domain does not include any additional antigen-binding domains and does not bind any other antigens. A subject BTTS may, in some instances, include only a single extracellular domain. Accordingly, an employed BTTS may be specific for a single antigen and only specific for the single antigen. Such, BTTSs may be referred to as a “single antigen BTTS”. In some instances, a “dual antigen BTTS” may be employed.

In some instances, a BTTS may have an extracellular domain that includes the first or second members of two or more specific binding pairs. For example, in some instances, a BTTS may have an extracellular domain that includes a first antigen-binding domain and a second antigen-binding domain that are different such that the extracellular domain is specific for two different antigens. In some instances, a BTTS may have two or more extracellular domains that each includes the first or second members of two different specific binding pairs. For example, in some instances, a BTTS may have a first extracellular domain that includes a first antigen-binding domain and a second extracellular domain that includes a second antigen-binding domain where the two different antigen binding domains are each specific for a different antigen. As such, the BTTS may be specific for two different antigens.

A BTTS specific for two or more different antigens, containing either two extracellular domains or one extracellular domain specific for two different antigens, may be configured such that the binding of either antigen to the BTTS is sufficient to trigger activation of the BTTS, e.g., proteolytic cleavage of a cleavage domain of the BTTS, e.g., releasing an intracellular domain of the BTTS. Such a BTTS, capable of being triggered by any of two or more antigens, may find use in the described circuits as a component of a logic gate containing OR functionality. In some instances, a BTTS specific for two different antigens may be referred to as a “two-headed BTTS” or a tandem BTTS (or tanBTTS). For example, in some instances, a synNotch BTTS configured to bind two or more different antigens may be referred to as a tandem SynNotch or tanSynNotch. BTTS specific for multiple antigens will not be limited to only two antigens and may, e.g., be specific for and/or triggered by more than two antigens, including e.g., three or more, four or more, five or more, etc.

### Methods of Making

The present disclosure further includes methods of making the nucleic acids, circuits, and cells employed in the herein described methods. In making the subject nucleic acids and circuits, and components thereof, any convenient methods of nucleic acid manipulation, modification and amplification (e.g., collectively referred to as “cloning”) may be employed. In making the subject cells, containing the nucleic acids encoding the described circuits, convenient methods of transfection, transduction, culture, etc., may be employed.

A nucleotide sequence encoding all or a portion of the components of a circuit of the present disclosure can be present in an expression vector and/or a cloning vector. Where a subject circuit or component thereof is split between two or more separate polypeptides, nucleotide sequences encoding the two or more polypeptides can be cloned in the same or separate vectors. An expression vector can include a selectable marker, an origin of replication, and other features that provide for replication and/or maintenance of the vector. Suitable expression vectors include, e.g., plasmids, viral vectors, and the like.

Large numbers of suitable vectors and promoters are known to those of skill in the art;

many are commercially available for generating a subject recombinant construct. The following vectors are provided by way of example. Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

As noted above, in some embodiments, a nucleic acid comprising a nucleotide sequence encoding a circuit or component thereof of the present disclosure will in some embodiments be DNA or RNA, e.g., in vitro synthesized DNA, recombinant DNA, in vitro synthesized RNA, recombinant RNA, etc. Methods for in vitro synthesis of DNA/RNA are known in the art; any known method can be used to synthesize DNA/RNA comprising a desired sequence. Methods for introducing DNA/RNA into a host cell are known in the art. Introducing DNA/RNA into a host cell can be carried out in vitro or ex vivo or in vivo. For example, a host cell (e.g., an NK cell, a cytotoxic T lymphocyte, etc.) can be transduced, transfected or electroporated in vitro or ex vivo with DNA/RNA comprising a nucleotide sequence encoding all or a portion of a circuit of the present disclosure.

Methods of the instant disclosure may further include culturing a cell genetically modified to encode a circuit of the instant disclosure including but not limited to e.g., culturing the cell prior to administration, culturing the cell in vitro or ex vivo (e.g., the presence or absence of one or more antigens), etc. Any convenient method of cell culture may be employed whereas such methods will vary based on various factors including but not limited to e.g., the type of cell being cultured, the intended use of the cell (e.g., whether the cell is cultured for research or therapeutic purposes), etc. In some instances, methods of the instant disclosure may further include common processes of cell culture including but not limited to e.g., seeding cell cultures, feeding cell cultures, passaging cell cultures, splitting cell cultures, analyzing cell cultures, treating cell cultures with a drug, harvesting cell cultures, etc.

Methods of the instant disclosure may, in some instances, further include receiving and/or collecting cells that are used in the subject methods. In some instances, cells are collected from a subject. Collecting cells from a subject may include obtaining a tissue sample from the subject and enriching, isolating and/or propagating the cells from the tissue sample. Isolation and/or enrichment of cells may be performed using any convenient method including e.g., isolation/enrichment by culture (e.g., adherent culture, suspension culture, etc.), cell sorting (e.g., FACS, microfluidics, etc.), and the like. Cells may be collected from any convenient cellular tissue sample including but not limited to e.g., blood (including e.g., peripheral blood, cord blood, etc.), bone marrow, a biopsy, a skin sample, a cheek swab, etc. In some instances, cells are received from a source including e.g., a blood bank, tissue bank, etc. Received cells may have been previously isolated or may be received as part of a tissue sample thus isolation/enrichment may be performed after receiving the cells and prior to use. In certain instances, received cells may be non-primary cells including e.g., cells of a cultured cell line. Suitable cells for use in the herein described methods are further detailed herein.

### Nucleic Acids

As summarized above, the present disclosure provides nucleic acids encoding a circuit for treating a subject for a disease or disorder.

Recombinant expression vectors of the present disclosure include those comprising one or more of the described nucleic acids. A nucleic acid comprising a nucleotide sequence encoding all or a portion of the components of a circuit of the present disclosure will in some embodiments be DNA, including, e.g., a recombinant expression vector. A nucleic acid comprising a nucleotide sequence encoding all or a portion of the components of a circuit of the present disclosure will in some embodiments be RNA, e.g., in vitro synthesized RNA.

As summarized above, in some instances, the subject circuits may make use of an encoding nucleic acid (e.g., a nucleic acid encoding a BTTS or an antigen-specific therapeutic) that is operably linked to a regulatory sequence such as a transcriptional control element (e.g., a promoter; an enhancer; etc.). In some cases, the transcriptional control element is inducible. In some cases, the transcriptional control element is constitutive. In some cases, the promoters are functional in eukaryotic cells. In some cases, the promoters are cell type-specific promoters. In some cases, the promoters are tissue-specific promoters.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoter and enhancer elements are known in the art. For expression in a bacterial cell, suitable promoters include, but are not limited to, lacI, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters.

In some instances, a transcriptional control element of a herein described nucleic acid may include a cis-acting regulatory sequence. Any suitable cis-acting regulatory sequence may find use in the herein described nucleic acids. For example, in some instances a cis-acting regulatory sequence may be or include an upstream activating sequence or upstream activation sequence (UAS). In some instances, a UAS of a herein described nucleic acid may be a Gal4 responsive UAS.

Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, the promoter is an immune cell promoter such as a CD8 cell-specific promoter, a CD4 cell-specific promoter, a neutrophil-specific promoter, or an NK-specific promoter. For example, a CD4 gene promoter can be used; see, e.g., Salmon et al. (1993) Proc. Natl. Acad. Sci. USA 90: 7739; and Marodon et al. (2003) Blood 101:3416. As another example, a CD8 gene promoter can be used. NK cell-specific expression can be achieved by use of an Ncr1 (p46) promoter; see, e.g., Eckelhart et al. (2011) Blood 117:1565.

In some instances, an immune cell specific promoter of a nucleic acid of the present disclosure may be a promoter of a B29 gene promoter, a CD14 gene promoter, a CD43 gene promoter, a CD45 gene promoter, a CD68 gene promoter, a IFN-β gene promoter, a WASP gene promoter, a T-cell receptor (3-chain gene promoter, a V9 γ (TRGV9) gene promoter, a V2 δ (TRDV2) gene promoter, and the like.

In some cases, a nucleic acid comprising a nucleotide sequence encoding a circuit of the present disclosure, or one or more components thereof, is a recombinant expression vector or is included in a recombinant expression vector. In some embodiments, the recombinant expression vector is a viral construct, e.g., a recombinant adeno-associated virus (AAV) construct, a recombinant adenoviral construct, a recombinant lentiviral construct, a recombinant retroviral construct, etc. In some cases, a nucleic acid comprising a nucleotide sequence encoding a circuit of the present disclosure, or one or more components thereof, is a recombinant lentivirus vector. In some cases, a nucleic acid comprising a nucleotide sequence encoding a circuit of the present disclosure, or one or more components thereof, is a recombinant AAV vector.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., Hum Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, the vector is a lentivirus vector. Also suitable are transposon-mediated vectors, such as piggyback and sleeping beauty vectors.

In some instances, nucleic acids of the present disclosure may have a single sequence encoding two or more polypeptides where expression of the two or more polypeptides is made possible by the presence of a sequence element between the individual coding regions that facilitates separate expression of the individual polypeptides. Such sequence elements, may be referred to herein as bicistronic-facilitating sequences, where the presence of a bicistronic-facilitating sequence between two coding regions makes possible the expression of a separate polypeptide from each coding region present in a single nucleic acid sequence. In some instances, a nucleic acid may contain two coding regions encoding two polypeptides present in a single nucleic acid with a bicistronic-facilitating sequence between the coding regions. Any suitable method for separate expression of multiple individual polypeptides from a single nucleic acid sequence may be employed and, similarly, any suitable method of bicistronic expression may be employed.

In some instances, a bicistronic-facilitating sequence may allow for the expression of two polypeptides from a single nucleic acid sequence that are temporarily joined by a cleavable linking polypeptide. In such instances, a bicistronic-facilitating sequence may include one or more encoded peptide cleavage sites. Suitable peptide cleavage sites include those of self-cleaving peptides as well as those cleaved by a separate enzyme. In some instances, a peptide cleavage site of a bicistronic-facilitating sequence may include a furin cleavage site (i.e., the bicistronic-facilitating sequence may encode a furin cleavage site).

In some instances, the bicistronic-facilitating sequence may encode a self-cleaving peptide sequence. Useful self-cleaving peptide sequences include but are not limited to e.g., peptide 2A sequences, including but not limited to e.g., the T2A sequence.

In some instances, a bicistronic-facilitating sequence may include one or more spacer encoding sequences. Spacer encoding sequences generally encode an amino acid spacer, also referred to in some instances as a peptide tag. Useful spacer encoding sequences include but are not limited to e.g., V5 peptide encoding sequences, including those sequences encoding a V5 peptide tag.

Multi- or bicistronic expression of multiple coding sequences from a single nucleic acid sequence may make use of but is not limited to those methods employing furin cleavage, T2A, and V5 peptide tag sequences. For example, in some instances, an internal ribosome entry site (IRES) based system may be employed. Any suitable method of bicistronic expression may be employed including but not limited to e.g., those described in Yang et al. (2008) Gene Therapy. 15(21):1411-1423; Martin et al. (2006) BMC Biotechnology. 6:4; the disclosures of which are incorporated herein by reference in their entirety.

### Cells

As summarized above, the present disclosure also provides immune cells. Immune cells of the present disclosure include those that contain one or more of the described nucleic acids, expression vectors, etc., encoding a described circuit. Immune cells of the present disclosure include mammalian immune cells including e.g., those that are genetically modified to produce the components of a circuit of the present disclosure or to which a nucleic acid, as described above, has been otherwise introduced. In some instances, the subject immune cells have been transduced with one or more nucleic acids and/or expression vectors to express one or more components of a circuit of the present disclosure.

Suitable mammalian immune cells include primary cells and immortalized cell lines.

Suitable mammalian cell lines include human cell lines, non-human primate cell lines, rodent (e.g., mouse, rat) cell lines, and the like. In some instances, the cell is not an immortalized cell line, but is instead a cell (e.g., a primary cell) obtained from an individual. For example, in some cases, the cell is an immune cell, immune cell progenitor or immune stem cell obtained from an individual. As an example, the cell is a lymphoid cell, e.g., a lymphocyte, or progenitor thereof, obtained from an individual. As another example, the cell is a cytotoxic cell, or progenitor thereof, obtained from an individual. As another example, the cell is a stem cell or progenitor cell obtained from an individual.

As used herein, the term “immune cells” generally includes white blood cells (leukocytes) which are derived from hematopoietic stem cells (HSC) produced in the bone marrow. “Immune cells” includes, e.g., lymphoid cells, i.e., lymphocytes (T cells, B cells, natural killer (NK) cells), and myeloid-derived cells (neutrophil, eosinophil, basophil, monocyte, macrophage, dendritic cells). “T cell” includes all types of immune cells expressing CD3 including T-helper cells (CD4+ cells), cytotoxic T-cells (CD8+ cells), T-regulatory cells (Treg) and gamma-delta T cells. A “cytotoxic cell” includes CD8+ T cells, natural-killer (NK) cells, and neutrophils, which cells are capable of mediating cytotoxicity responses. “B cell” includes mature and immature cells of the B cell lineage including e.g., cells that express CD19 such as Pre B cells, Immature B cells, Mature B cells, Memory B cells and plasmablasts. Immune cells also include B cell progenitors such as Pro B cells and B cell lineage derivatives such as plasma cells.

Immune cells encoding a circuit of the present disclosure may be generated by any convenient method. Nucleic acids encoding one or more components of a subject circuit may be stably or transiently introduced into the subject immune cell, including where the subject nucleic acids are present only temporarily, maintained extrachromosomally, or integrated into the host genome. Introduction of the subject nucleic acids and/or genetic modification of the subject immune cell can be carried out in vivo, in vitro, or ex vivo.

In some cases, the introduction of the subject nucleic acids and/or genetic modification is carried out ex vivo. For example, a T lymphocyte, a stem cell, or an NK cell is obtained from an individual; and the cell obtained from the individual is modified to express components of a circuit of the present disclosure. The modified cell can thus be redirected to one or more antigens of choice, as defined by the one or more antigen binding domains present on the introduced components of the circuit. In some cases, the modified cell is modulated ex vivo. In other cases, the cell is introduced into (e.g., the individual from whom the cell was obtained) and/or already present in an individual; and the cell is modulated in vivo, e.g., by administering a nucleic acid or vector to the individual in vivo.

### Circuits

As summarized above, the present disclosure also provides circuits encoded by nucleic acid sequences, also referred to in some instances as molecular circuits. Such circuits may, in some instances, be present and/or configured in expression vectors and/or expression cassettes. The subject nucleic acids of the present circuits may, in some instances, be contained within a vector, including e.g., viral and non-viral vectors. Such circuits may, in some instances, be present in cells, such as immune cells, or may be introduced into cells by various means, including e.g., through the use of a viral vector. Cells may, in some instances, be genetically modified to encode a subject circuit, where such modification may be effectively permanent (e.g., integrated) or transient as desired.

Encoded components of the circuits of the present disclosure will generally include at a minimum at least two encoded BTTSs and at least one encoded therapeutic protein. The expression of a component of a circuit of the present disclosure may be dependent upon the state (i.e., active/inactive state) of another component of the circuit. For example, the expression of the therapeutic may be dependent upon the activation of a BTTS, where the BTTS is activated by binding to an antigen for which the BTTS is specific. In some instances, dependency of one component of the circuit on another may be mediated by a regulatory sequence. For example, a sequence encoding a second component of a circuit may be operably linked to a regulatory sequence that is responsive to the activation of a first component of the circuit, thus linking the expression of the second component to the activation of the first.

The use of a BTTS in a circuit of the present disclosure facilitates the linking of expression and/or activity to molecular binding events. Systems involving binding-triggered transcriptional switches, and components thereof, have been described in PCT Publication No. WO 2016/138034, US Patent Application Pub. No. US 2016-0264665 A1 and issued U.S. Pat. Nos. 9,670,281 and 9,834,608; the disclosures of which are incorporated by reference herein in their entirety.

Circuits of the present disclosure are configured as an “AND” gate, where “AND” gates include where two or more inputs are required for propagation of a signal. For example, in some instances, an AND gate allows signaling through a first input of a first polypeptide or a first polypeptide domain and a second input dependent upon the output of the first input. In an AND gate two inputs, e.g., two antigens, are required for signaling through the circuit.

### KITS

The present disclosure provides a kit for carrying out a method as described herein and/or constructing one or more circuits, components thereof, nucleic acids encoding a circuit or a component thereof, etc. In some cases, a subject kit comprises a vector, e.g., one or more expression vectors or a delivery vectors, comprising a nucleotide sequence encoding a circuit of the present disclosure or one or more portions thereof. Delivery vectors may be provided in a delivery device or may be provided separately, e.g., as a kit that includes the delivery vector and the delivery device as separate components of the kit.

In some cases, a subject kit comprises a cell, e.g., a host cell or host cell line, that is or is to be genetically modified with a nucleic acid comprising nucleotide sequence encoding a circuit of the present disclosure or a portion thereof. In some cases, a subject kit comprises a cell, e.g., a host cell, that is or is to be genetically modified with a recombinant expression vector comprising a nucleotide sequence encoding a circuit of the present disclosure. Kit components can be in the same container, or in separate containers.

Any of the above-described kits can further include one or more additional reagents, where such additional reagents can be selected from: a dilution buffer; a reconstitution solution; a wash buffer; a control reagent; a control expression vector; a nucleic acid encoding a negative control (e.g., a circuit that lacks the one or more elements); a nucleic acid encoding a positive control polypeptide; and the like.

In addition to above-mentioned components, a subject kit can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### RESULTS

Pipeline for Identifying Antigen Combinations that Improve Tumor Discrimination

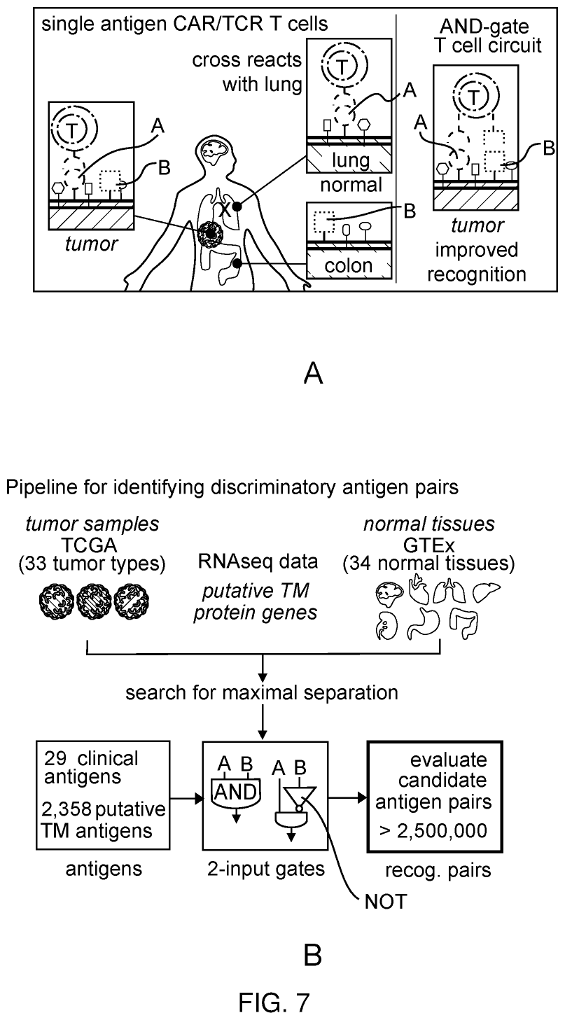

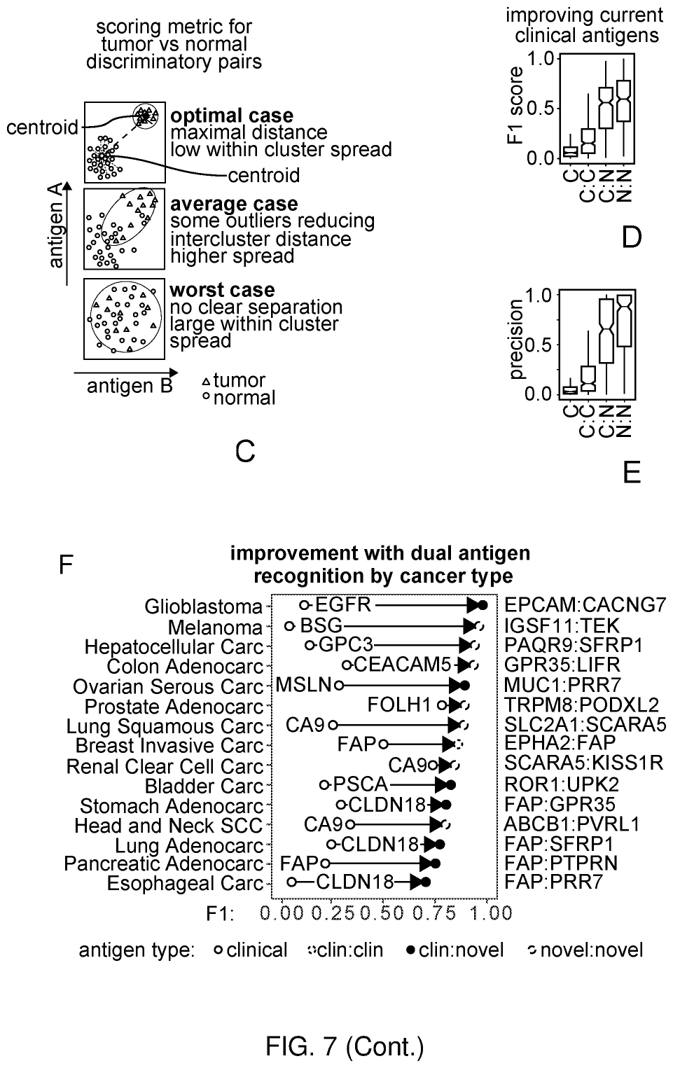

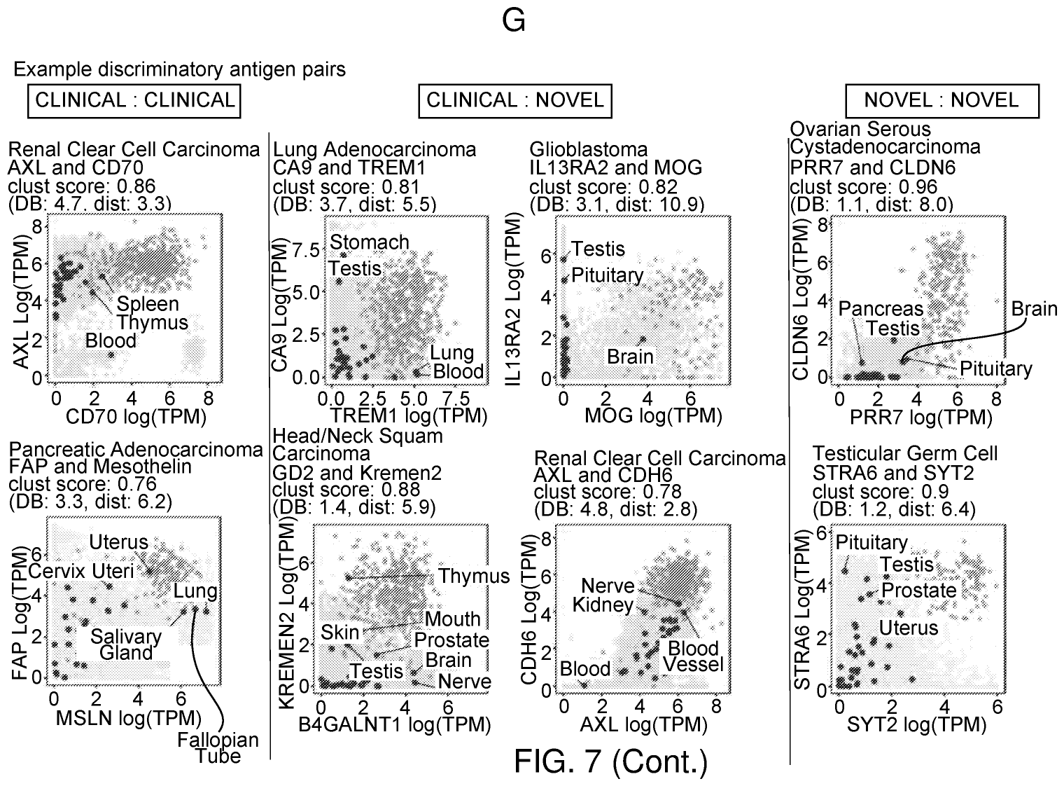

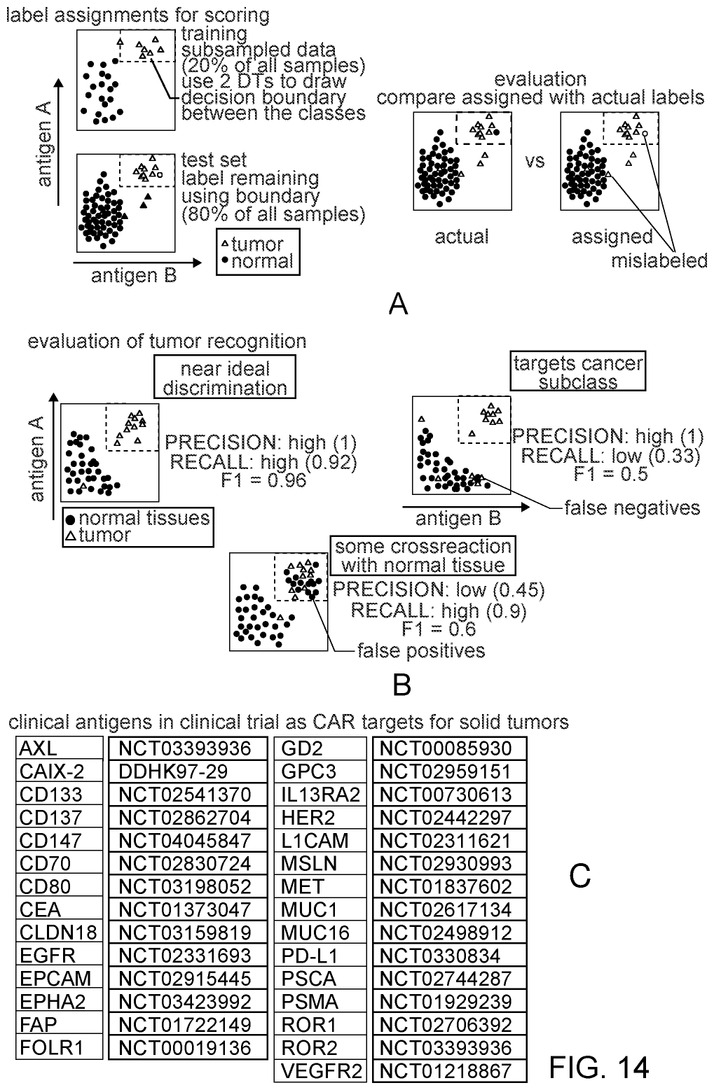

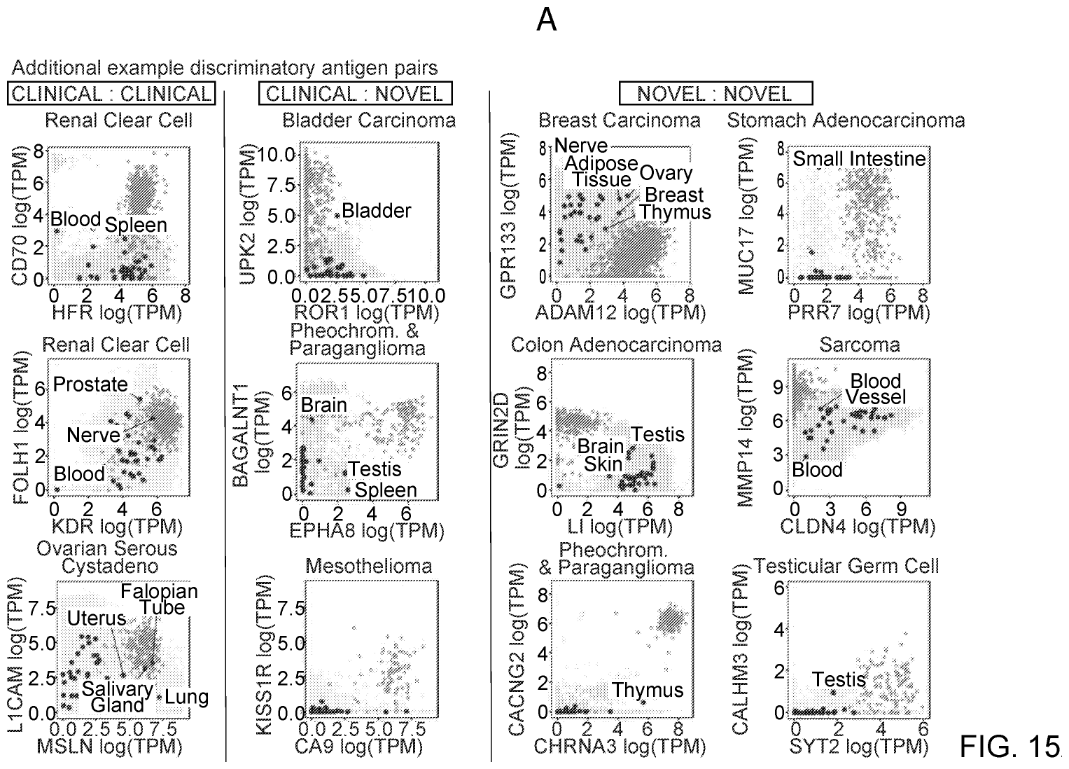

The space of approximately 2,400 surface expressed genes for AND and NOT gate combinations across 33 tumor types were searched for pairs that optimally distinguish a given tumor type's samples from 34 normal tissue samples (FIG. 7B) (Supplemental file tumor_and_tissue_sample_counts.xlsx). Annotations from the COMPARTMENTS database (Binder et al., 2014) were used to identify predicted transmembrane proteins as expressed on the plasma membrane. Of these, potential target antigens were classified as either: “clinical”—involved as a target of a CAR T cell therapy in a currently registered clinical trial (29 genes; see FIG. 14C); or “novel”—not currently targeted in a known therapeutic T cell clinical trial. The RNAseq expression data across 9,084 samples taken from The Cancer Genome Atlas (https://www.cancer.gov/tcga) and 12,402 samples from Genotype Tissue Expression Project (GTEx) (Aguet et al., 2017) are used to measure the level of antigen gene expression. To reduce expression differences due to technical variation all samples were batch corrected, then looked for separation in log transformed TPM expression space.

Predictions were made to maximize the separation of each tumor type from all other normal tissues using a distance-based approach derived from cluster evaluation metrics (see methods) and used these clustering-based scores to rank all putative pairs per tumor type (FIG. 7C). To evaluate the top pairs identified by this method, classifiers using each of the top ranked antigen pairs as features were built and how well each pair would separate tumor samples from normal tissue samples (F₁ score, precision, and recall) was assessed. Both F₁ and precision are focused on here, as precision gives a measure of how well off-target toxicity is avoided (1 is perfect avoidance) and F₁ scores balance precision with recall (how many of the tumor samples can be targeted). F₁ scores also range from 0 to 1, with scores of 1 signifying perfect classification between cancer and normal tissues.

### Recognition of All Cancer Types Can Be Improved By Adding Secondary Antigens to Current Clinical CAR T Targets

Clustering-based scores for the current clinically targeted antigens described in (FIG. 14C) were first calculated. These single antigen scores were compared with those obtained for antigen pairs in which: two clinically targeted antigens (clinical antigens) are combined, a clinical antigen is combined with a novel putative surface antigen or two novel surface antigens are combined. As the majority of antigen combinations are poor discriminators between cancer and normal tissues, the best single clinical antigens for each cancer type are summarized, and similarly, the top 10 antigen pairs from each type of combination (e.g. clinical-clinical) per cancer type (Supplemental File top-10-clins-and-pairs.xlsx) as ranked by their clustering-based scores are summarized.

To give more insight into how these top antigens separate normal vs tumor samples, classifiers were built for each and their performance was plotted. Classification scores (F₁ and precision) are summarized across cancer types to see the effects of adding additional secondary antigens. As shown in FIGS. 7D and 7E, the current clinical antigens on average lack sensitivity and specificity, when used as the sole recognition antigen (μ F₁=0.09), however combining two clinical antigens with AND or NOT logic for tumor recognition leads to significant improvement in both precision and recall as seen by the jump in F₁ (μₜₒₚ₁₀ F₁=0.21; ranksum p=4.14×10⁻²⁰). Excitingly, the addition of a novel antigen to a clinical antigen for tumor recognition allows for even more improvement in discrimination (μ_{top 0} F₁=0.52; ranksum p=2.08×10⁻⁸³), with novel-novel pairings showing even more potential (μₜₒₚ₁₀ F₁=0.57). Furthermore, the substantially improved precision of novel-novels (μₜₒₚ₁₀ F₁=0.72) in FIG. 7E suggests there are avenues for effectively limiting cross-reactivity with only two antigens for most tumor types. Taken together these results suggest that discrimination achievable by current clinical antigens can be dramatically improved by incorporating them into antigen pairs recognized by Boolean gated T cells.

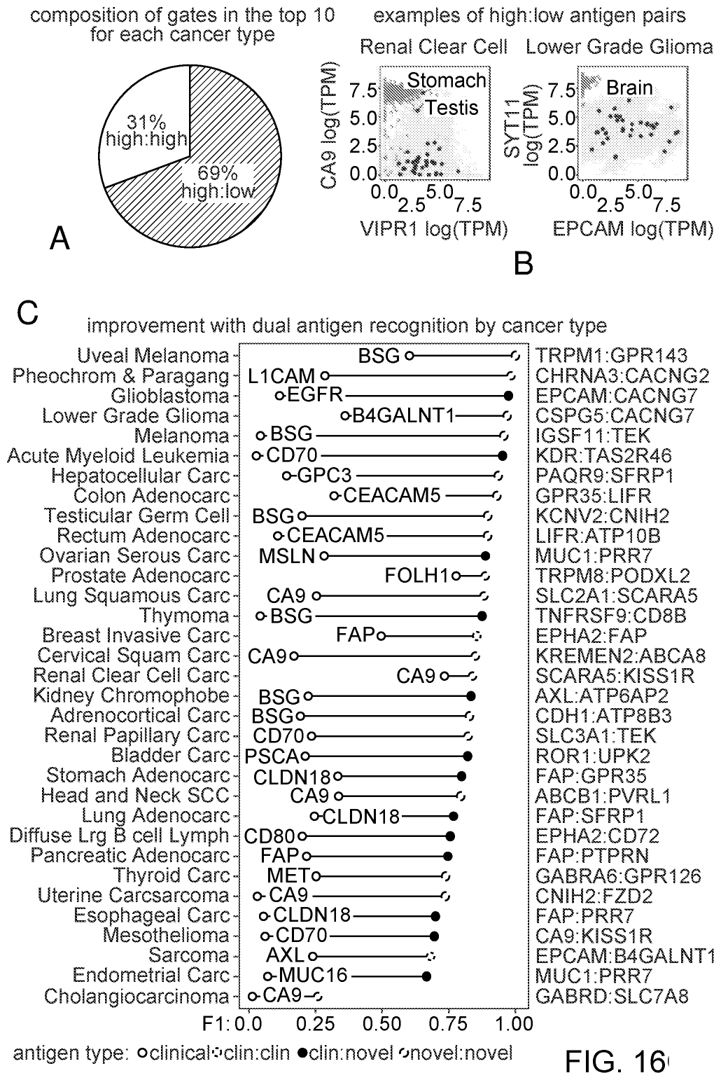

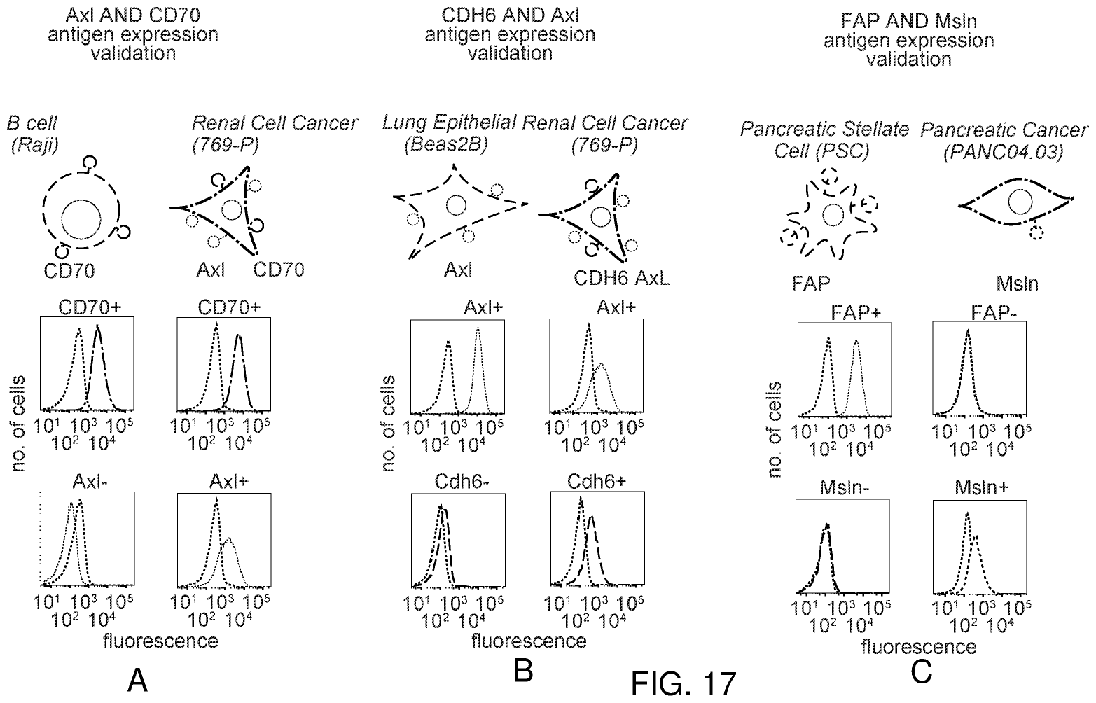

The top combinatorial improvement in tumor discrimination scores across 33 individual cancer types (FIGS. 7F, 16C) was also analyzed. Nearly all of the cancers examined showed marked improvement from the best clinical antigen to the best double antigen (μ ΔF₁=0.59). More specifically, among these top pairs reductions in overall cross-reactivity (μ Δprec=0.73) and an increase in sensitivity (μ Δrecall=0.15) were seen, with clinical-novel and novel-novel antigen pairs showing the best discrimination performance. Thus, targeting combinatorial antigen signatures are highly likely to help improve specificity of targeted treatments.

### Scope of Alternative Solutions for Discriminatory Antigen Pairs

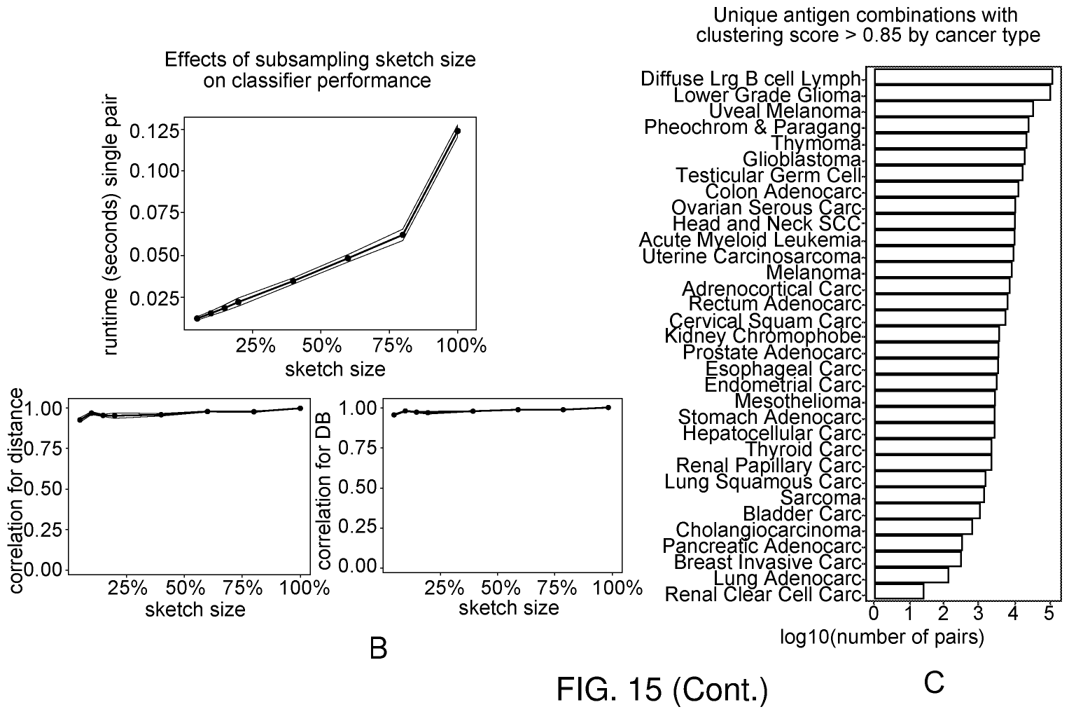

As shown in FIG. 7F certain tumor types are harder to distinguish from normal tissue. In order to assess this, unique antigen combinations that showed promising clustering-based scores above a threshold of 0.85 (FIG. 15C) were tallied. All cancers show at least several (>25) antigen pairs above this cutoff with many having thousands of strong pairs, suggesting a potential therapeutic avenue for all tumor types when using a pair of antigens.

### Examples of Antigen Pairs Predicted to Improve Tumor Recognition

Our top possible antigen pairs as ranked by clustering score and their ability to discriminate any given cancer type will be made available through an interactive webserver. The webserver allows users to score and generate a scatterplot for any possible transmembrane pair. In FIG. 7G, a few examples of high-performing antigen pairs (high clustering scores) are highlighted. These 2D scatterplots show RNAseq expression level of both antigens in samples of a particular cancer (red) and in normal tissue samples (light grey). Dark circles highlight the centroids each normal tissue type, indicated as labelled. In these plots, a high degree of separation is indicated by a cluster of red (cancer) points that are segregated away from the bulk of normal grey tissue samples. This segregation can occur in the upper right quadrant (high:high representing AND gate); in the upper left quadrant (low:high representing NOT gate), or lower right quadrant (high:low, NOT gate). The plots shown in FIG. 7G represent only a small fraction of possible high performing combinations. Other examples of high performing antigen pairs are shown in FIG. 15A, with all other examples accessible via the webserver. Some of these specific antigen pairs are discussed in the following sections.

Experimental Validation: Secondary Antigens that Improve CAR T Recognition of Renal Cell Carcinoma

This analysis provides a very large data-set of potential antigen pairs for clinical translation as AND gate CAR T cells, many of which are actionable using currently available antigen recognition domains. To outline how antigen pairs can be translated to cellular design and to validate the bioinformatic predictions, a pair of engineered cell designs capable of specifically recognizing renal cell carcinoma (RCC) was constructed. Two predicted examples of combinatorial antigens for RCC recognition are shown in FIG. 7G.

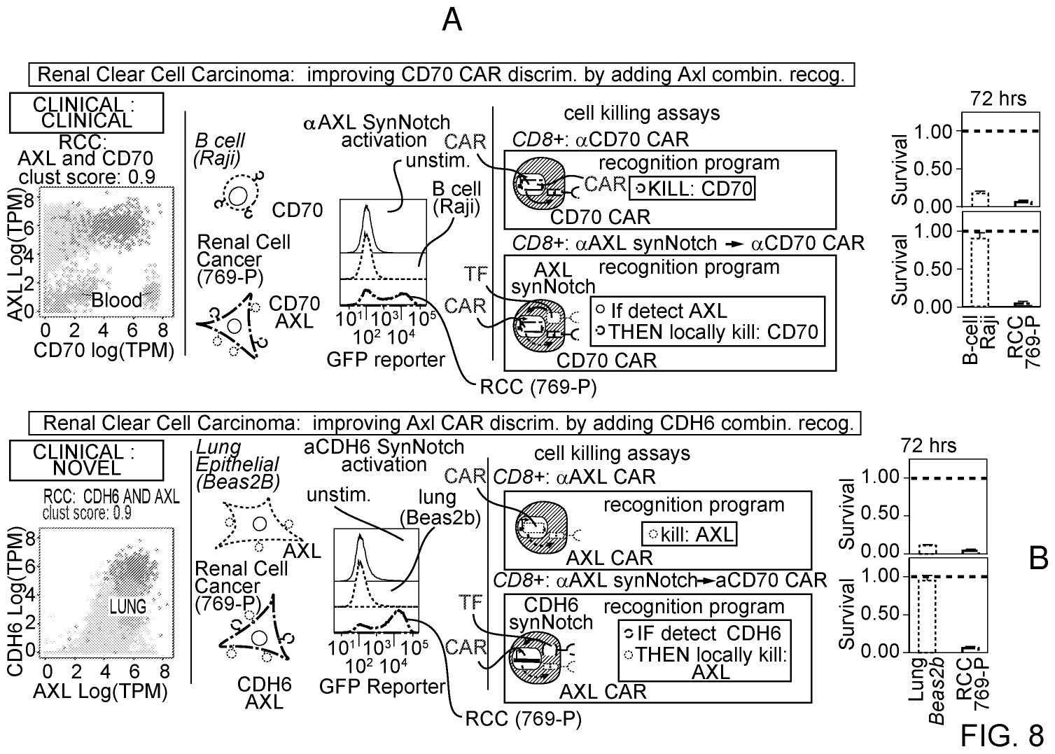

RCC is known to overexpress the tumor associated antigens CD70 and AXL (Jilaveanu et al., 2012; Yu et al., 2015), which was experimentally confirmed in an RCC cell line (769-P) (FIG. 17A). Both of these antigens are currently involved in CAR T trials. However, as single CAR targets they are imperfect. CD70 is also expressed on a number of blood cells, including activated T cells, germinal center B cells, and dendritic cells in lymph nodes (Hintzen et al., 1994; Tesselaar et al., 2003). AXL is also expressed in many normal tissues including the lung (Qu et al., 2016). Interestingly, however, it is found that the cross-reactive normal tissues for these two antigen targets are non-overlapping and, thus, the combination of these two complementary clinical antigen targets is predicted to greatly improve discrimination of tumor vs normal tissue (FIG. 8A).

To take advantage of this complementary pair of AND antigens for RCC, a CAR that recognizes CD70 using it's cognate binding partner CD27 as the recognition domain was engineered (Wang et al., 2017). In vitro cytotoxicity assays, showed that this CAR T cell was able to clear a renal cell carcinoma line (769-P) but also showed significant cytotoxicity against a B cell line (Raji cells). To create a T cell that recognizes AXL AND CD70, a synNotch receptor was first engineered (Morsut et al., 2016) using an α-AXL scFv recognition domain fused to the Notch transmembrane domain and an orthogonal transcription factor (GAL4-VP64). It was found that T cells expressing an α-AXL synNotch that are co-cultured with RCC cells activate a synNotch GFP reporter; in contrast, the same T cells co-cultured with Raji B cells, which do not express AXL, do not activate the AXL synNotch receptor. Then, AND gate T cells in which an α-AXL synNotch drives expression of a CD70 CAR were engineered. It is found that this AND circuit caused the specific lysis of RCC cells, but not of Raji B cells (FIG. 8A). Thus, the combinatorial recognition of AXL AND CD70 improves upon the CD70 single target CAR, allowing discrimination between RCC cells and B cells.

Similarly, the single target α-AXL CAR is by itself a potential treatment for RCC. However, as above targeting AXL is predicted to have toxic cross reactivity with lung tissue. An AXL CAR was constructed, and when expressed in human primary CD8+ T cells, it was found to have cytotoxic activity against both a RCC cell line and an immortalized lung epithelial cell line (Beas2B) (FIG. 8B). Based on the current bioinformatics analysis of combinatorial antigens, it was predicted that the novel antigen CDH6 (cadherin 6), which has not previously been used as cellular therapy target, would improve the precision of an AXL CAR (FIG. 8B). CDH6 is a protein that mediates calcium-dependent cell-cell adhesion with PAX8 lineage linked expression (de Cristofaro et al., 2016) in the fetal kidney (Mbalaviele et al., 1998) as well as proximal tubule epithelium and is over-expressed in renal and ovarian cancer (Paul et al., 1997). A synNotch receptor targeting CDH6 was generated by screening four potential CDH6 scFv's fused to the synthetic notch core receptor. It was found that α-CDH6 synNotch receptors expressed in human primary T cells would specifically drive GFP reporter activity when co-cultured with an RCC cell line, but not with CDH6 negative lung epithelium cells (Beas2B). When an AND-gate T cell with α-CDH6 synNotch driving expression of an α-AXL CAR was constructed, it was found that specific lysis was only seen for the RCC cell line, and not the lung epithelial cell line. Thus, the combinatorial recognition of CDH6 and AXL improves upon the AXL single target CAR in that allows discrimination between RCC cells and lung epithelial cells.

These two examples show that there are multiple ways to improve recognition of a specific cancer like RCC by harnessing combinatorial antigen recognition. In total, the analysis predicts 25 antigen pairs that discriminate RCC from normal tissues with a clustering score of >0.85 (FIG. 15C). This set of experiments, illustrates a pipeline by which improved combinatorial CAR T circuits can be computationally identified and validated.

### Recognizing Antigen Combinations Presented on Different Cells: Trans-Recognition Allows Integration of Information Across Multiple Cells in a Tumor

One of the features of bulk RNA sequencing data is that it includes expression data from malignant cells as well as adjacent non-malignant cells that together make up the tumor ecosystem. In fact, several of the antigen pairs highlighted in FIG. 7G include antigens that are thought to be expressed on different cells within the tumor. For example, in lung adenocarcinoma, the antigen CA9 is expressed in the cancer cells, while the complementary antigen, triggering receptor expressed on myeloid cells 1 (TREM1), is likely found on the myeloid cells of the tumor microenvironment (Roe et al., 2014). Similarly, in pancreatic cancer, the antigen Mesothelin is expressed on the cancer cells, while a predicted complementary antigen, fibroblast activation protein (FAP), is likely expressed on stromal cells (fibroblasts and myofibroblasts) (Kraman et al., 2010). Finally, in glioblastoma, the antigen IL13Ra2 is expressed in cancer cells, but a predicted complementary antigen, myelin oligodendrocyte glycoprotein (MOG), is likely expressed on the oligodendrocytes of the brain that form the myelin sheath around neurons (Solly et al., 1996).

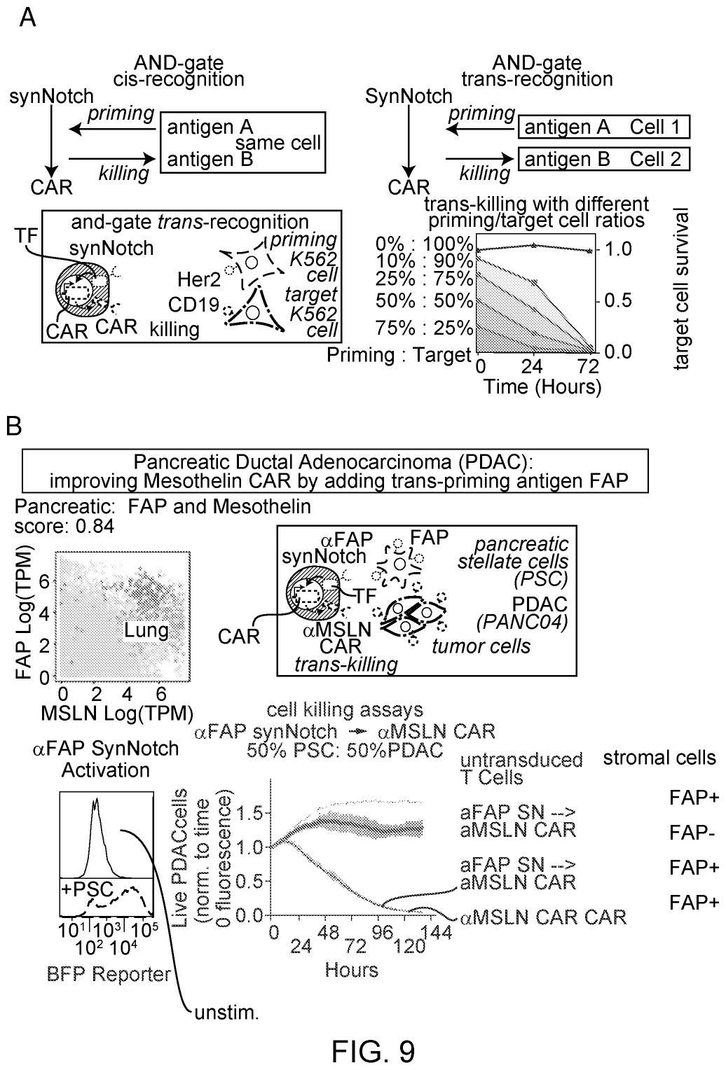

Thus, the need to define two different situations for combinatorial antigen recognition was determined: in cis-recognition of the pair of combinatorial antigens are expressed on the same (cancer) cell, while in trans-recognition the pair of antigens are expressed on two different cells in the same tumor environment (FIG. 9A). For antigens on two distinct cells within the tumor ecosystem to be actionable, the therapeutic T cells needs to be capable of trans-recognition.

It was hypothesized that the cellular circuits described herein that use a SynNotch receptor to prime the expression of a killing receptor (CAR) might be able to perform trans-recognition. To test this hypothesis, human primary CD8+ T cells in which an α-Her2 synNotch receptor drives expression of an α-CD19 CAR were constructed. These T cells were co-cultured with one population of K562 cells engineered to express the priming antigen (Her2) and a separate population of K562 cells engineered to express the target killing antigen (CD19). These AND-gate T cells showed no killing activity against a pure population of CD19+ K562 target cells, indicating that they require a priming signal. However, mixtures of priming and target cells that contain as low as 10% priming cells show killing and clearance of the target cells, albeit with slower kinetics with lower priming cell ratios (FIG. 9A). This result shows that synNotch→CAR “prime-and-kill” circuits have the capability to achieve trans-recognition of antigen pairs—they can prime off of one cell and kill a neighboring cell expressing the killing antigen (it is unclear if other AND gate schemes share this trans-killing capability because of mechanistic differences).

### Trans-Recognition of Mesothelin and FAP Antigen Pair in Pancreatic Cancer

The ability to construct CAR T cells with trans-recognition circuits opens new options to kill cancers based on priming off of adjacent normal tissue or tumor adjacent reactive stroma, as in some of the trans-recognition pairs highlighted in FIG. 7G. In pancreatic cancer, it was found that the tumor stromal antigen Fibroblast Activation Protein alpha (FAP) is predicted to improve the precision of recognition by α-Mesothelin (MSLN) CAR T cells. Mesothelin has been an attractive target used in a number of prior clinical trials (Beatty et al., 2014; Tanyi et al., 2016) because it is overexpressed in many tumors, including mesothelioma, pancreatic, lung and ovarian cancer; however, mesothelin is also expressed in a number of normal tissues, especially in the lung. α-MSLN CAR T cells are currently under clinical investigation but have been limited by dose-limiting pulmonary toxicity (Carl June, personal communication).

The combination of FAP and MSLN is predicted to improve the specificity of detection and minimize potential pulmonary cross-reactivity (FIG. 9B). To test this predicted recognition pair, first synNotch receptors were constructed that would specifically recognize FAP, testing a set of α-FAP scFV recognition domains. These α-FAP synNotch receptors were tested in human primary T cells and it was found that they showed inducible BFP reporter expression when co-cultured with immortalized pancreatic stellate cells (Hwang et al., 2008) validated to have high FAP expression (FIG. 17C). Combinatorial recognition T cells were then designed with a prime-and-kill AND gate circuit where FAP recognition by a synNotch receptor is used to induce expression of an α-MSLN CAR. As a model for pancreatic cancer the cell line Panc04.03 which expresses mesothelin (FIG. 17C) was used and it was found that these cells are killed by T cells expressing the single target α-MSLN CAR. The Panc04.03 cells, however, are not killed by the FAP-MSLN combinatorial recognition T cells unless the Panc04.03 target cells were co-cultured with the FAP+ pancreatic stellate cells, which serve to trans-prime the T cells (FIG. 9B).

This result shows how prime-and-kill combinatorial antigen circuits can be used to tap into and integrate antigen information on multiple cells in the tumor, including stromal, tissue, and immune cells that are characteristic of the disease microenvironment. This finding opens the door to different schemes of recognition that take advantage of the heterogeneous composition of tumors. The ability to achieve trans-recognition also provides potential ways to overcome the problem of recognizing heterogeneously expressed cancer antigens.

Linking synNotch Receptors and TCRs to Integrate External and Internal Antigen Recognition

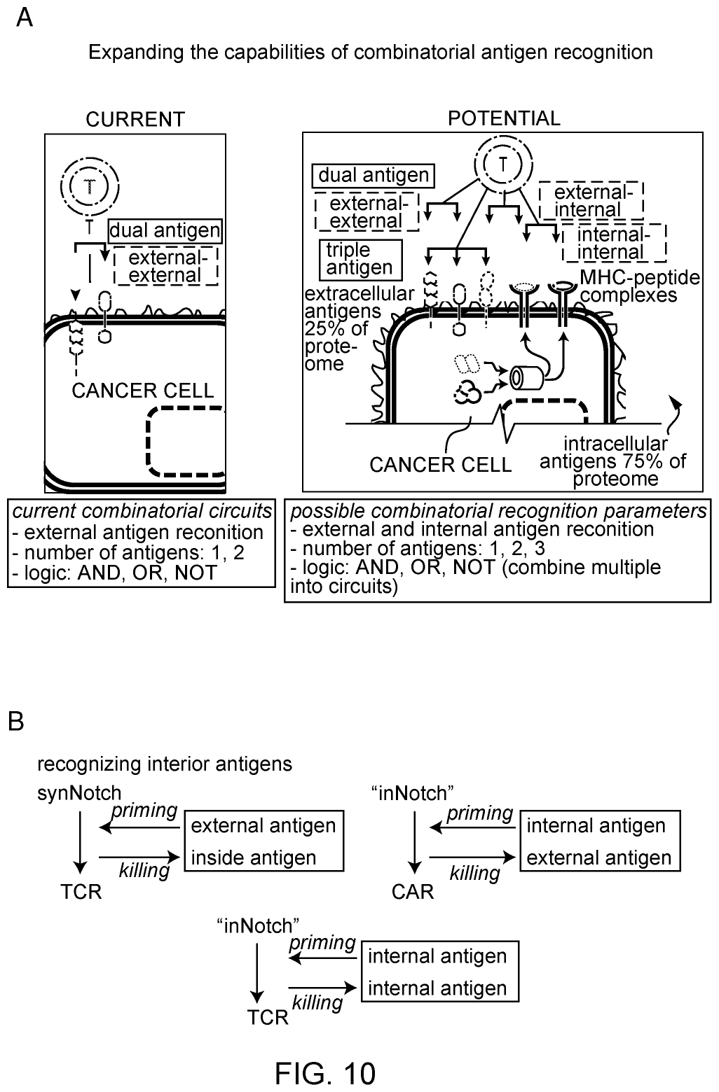

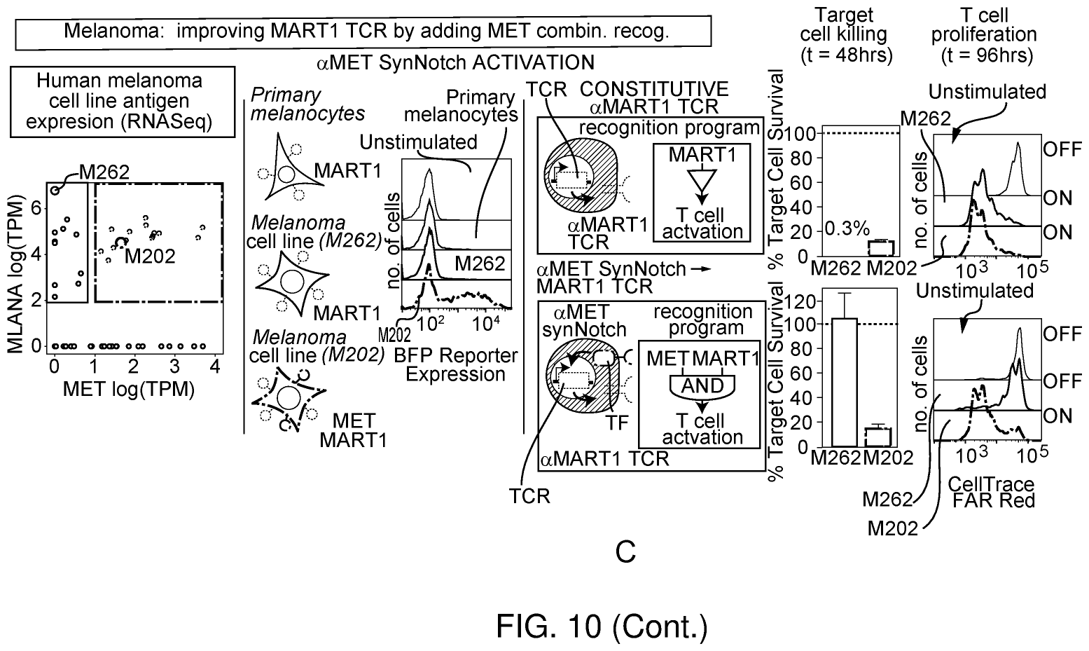

Given the demonstrated power of combinatorial recognition, exploring new and simple ways in which additional features of tumors could be integrated into recognition was desired. An ideal is to be able to engineer therapeutic T cells with a wide array of flexible programs capable of recognizing and exploiting the most discriminatory features of the tumor (FIG. 10A). Given that intracellular antigens represent ˜75% of the proteome (Almén et al., 2009) and are further enriched amongst cancer antigens (Vogelstein et al., 2013), it would be ideal to incorporate intracellular antigen sensing capabilities into AND gate T cells; however, previously published AND gate T cell strategies have been restricted to cell surface external antigens (Roybal et al., 2016a,b, Kloss et al. 2013). It was reasoned that synNotch receptors could also be used to induce expression of TCRs, which are able to recognize intracellular proteins in the form of peptides presented on major histocompatibility complexes (pMHC). This kind of external-internal AND gate could be useful to restrict the action of a promiscuous TCR to a specific subset of tissues, based on the expression of the priming (synNotch activating) antigen. Altogether, incorporating internal antigen recognition would dramatically increase the pool of possible antigen combinations to target (FIG. 10B).

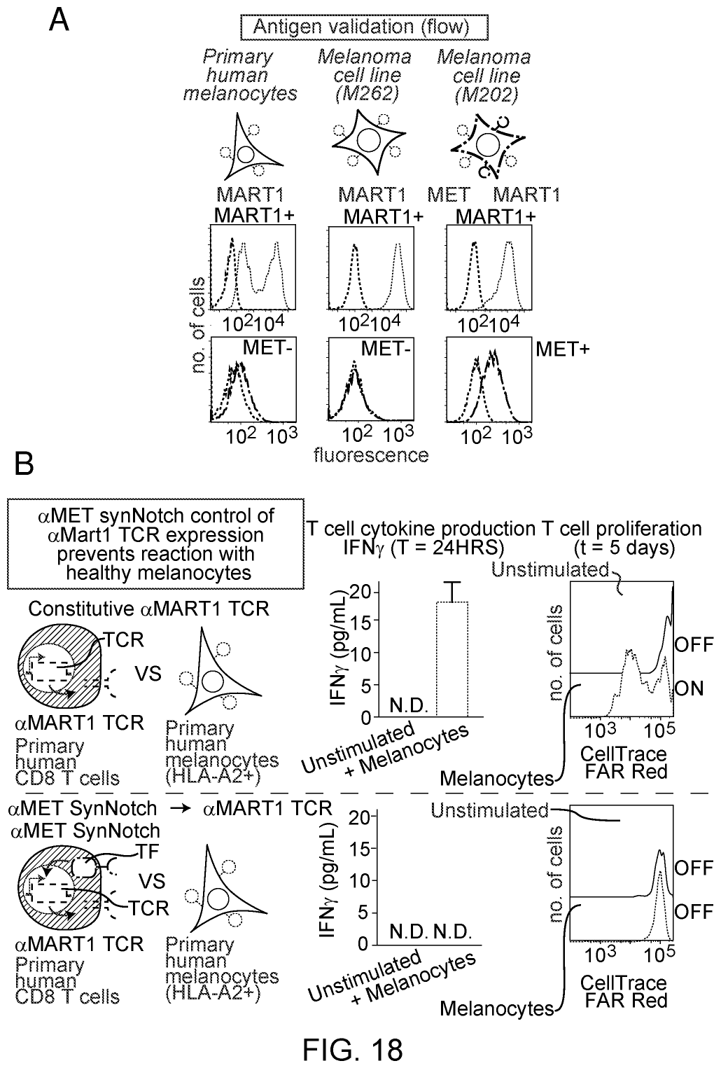

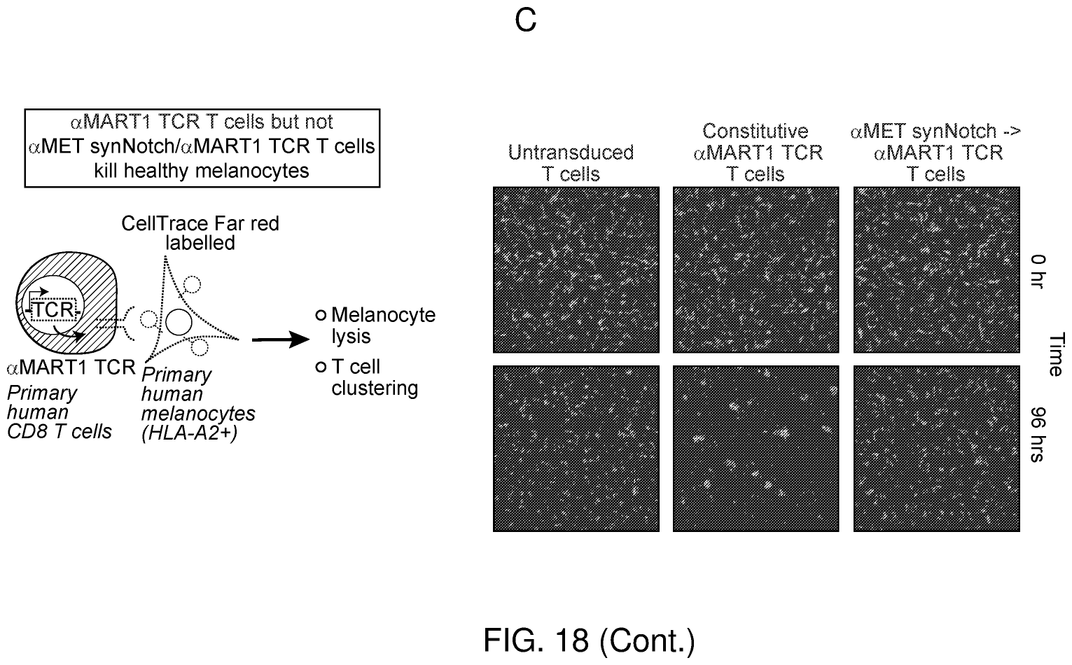

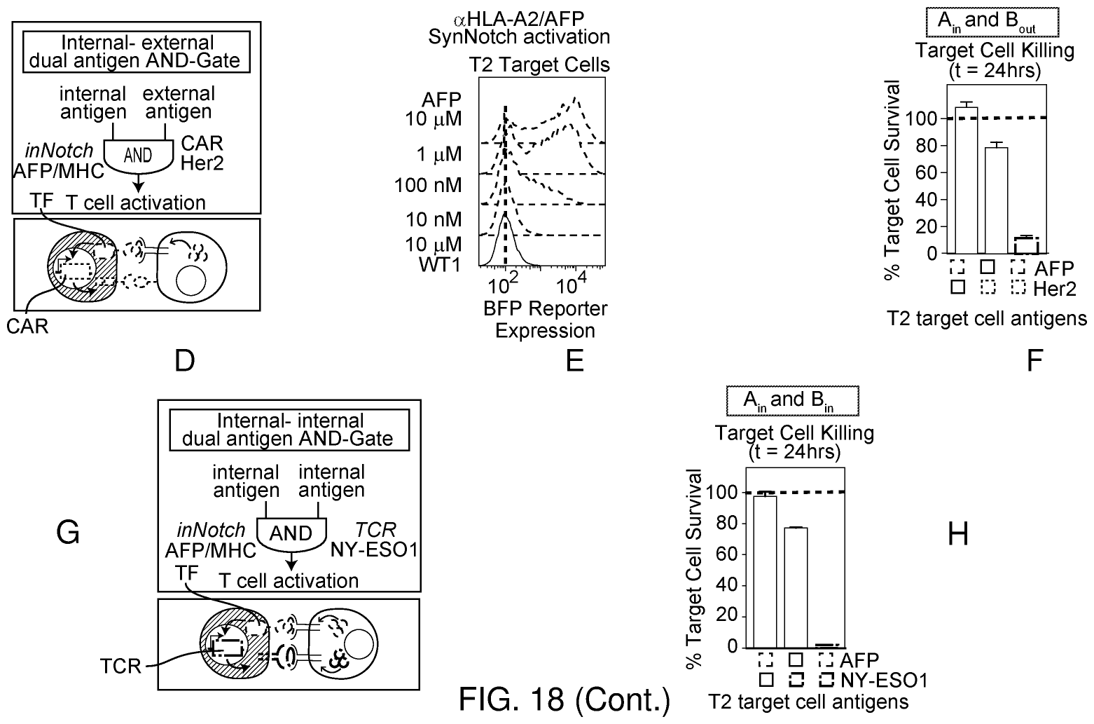

Moving toward clinically relevant systems involved focusing on engineered human primary CD8+ T cells with an external-internal antigen recognition circuit in which an □-MET synNotch receptor (external antigen) induced expression of an α-MART1 (HLA-A2) TCR (internal antigen; residue 27-35 peptide presented by HLA-A2) (FIG. 10C). MART1 stands for melanoma antigen recognized by T cells and is also known as MelanA (MLANA) (Johnson et al., 2006). This pair of antigens was focused on because of its potential clinical relevance for targeting melanoma. Prior clinical trials using α-MART1 TCRs to target melanoma have shown objective cancers responses. MART1, however, is expressed in normal melanocytes, and, not surprisingly, the α-MART1 TCR T cells showed dose-limiting toxic cross-reactivity with MART1 expressing healthy melanocytes in the skin, ear, and eye (Chodon et al., 2014; Johnson et al., 2009; Morgan et al., 2006). Thus, the ability to gate expression and activity of an α-MART1 TCR with a second antigen not expressed by normal melanocytes would be very useful. Unfortunately, there are relatively few normal skin samples in the collection of expression data used for this study. Therefore, instead expression data from a collection of human melanoma cell lines was profiled for differential expression of secondary antigens that could potentially be used in combination with MART1 to improve recognition for at least a subset of melanomas that can be discriminated from MART1 expressing healthy melanocytes. From this analysis, MET, a known melanoma-associated antigen (Puri et al., 2007), was identified as co-expressed with MART1 in many but not all melanoma cell lines and not by primary human melanocytes (FIG. 10C, FIG. 18A). Two HLA-A2+ melanoma cell lines of interest were specifically identified: M202 is MET+/MART1+, while M262 is MET−/MART1+ (i.e. analogous to primary melanocytes).

Targeting this combination of antigens involved first constructing a synNotch receptor with an α-MET nanobody. It was found that T cells expressing an α-MET synNotch would only activate expression of a BFP reporter in the presence of the MET+/MART1+ melanoma cell line M202. In contrast, no BFP reporter induction was observed in the presence of the MET−/MART1+ melanoma cell line M262 or primary human melanocytes (FIG. 10C). Then, human primary T cells with an AND gate circuit in which α-MET synNotch controls expression of an α-MART1 TCR were engineered. These engineered cells showed robust AND gate behavior and efficiently were activated by and killed the MET+/MART1+ melanoma cell line M202, while sparing primary human melanocytes and the MET−/MART1+ melanoma cell line M262 (a proxy for healthy skin cells) (FIG. 10C, FIG. 18B,C). In contrast, T cells engineered to constitutively express an α-MART1 TCR were activated by and killed all MART1+ target cells, including primary human melanocytes, as expected based on melanocyte toxicity seen in clinical trials with this TCR. T cells expressing the MET AND MART1 circuit killed MET+/MART1+ melanoma cells (M202) as efficiently as constitutive α-MART1 TCR T cells while not cross-reacting with MET−/MART1+ melanocytes and similar model cell lines. In summary, the synNotch→TCR circuits appear to be a robust system for engineering AND gate T cells to target external→internal antigen pairs.

synNotch Receptors Can Be Engineered to Recognize Internal Tumor-Associated Antigens as Peptide:MHCs

Multiple avenues for incorporating intracellular antigens into combinatorial recognition circuits were explored. In addition to using a synNotch to drive a TCR, synNotch receptors that use scFv's that recognize peptide:MHC complexes (pMHCs) as a way to prime on internal antigen peptides were also constructed. These internal synNotch receptors are referred to as internal synNotch or “inNotch” receptors (FIG. 9B). An α-HLA-A2/alpha-fetoprotein (AFP) inNotch receptor was constructed by using an scFv developed to recognize the 158-166 peptide derived from the liver cancer antigen AFP complexed with HLA-A2 (Liu et al., 2017). Human primary CD8+ T cells with an α-AFP inNotch receptor and a BFP reporter were first engineered, and it was found that these T cells only activated BFP reporter expression in the presence of target cells presenting AFP, while showing no activation in the presence of high levels of an off-target pMHC antigen (FIG. 18E). Then whether an α-AFP inNotch receptor could be incorporated into AND gates regulating either CAR or TCR expression was tested. T cells engineered with an α-AFP inNotch controlling either an α-Her2 CAR or α-HLA-A2/NY-ESO1 TCR acted as AND gates and specifically killed the intended dual-positive target cells (FIGS. 18D-H). Altogether, these results show that it is possible to incorporate one or two internal antigens into dual antigen AND gate circuits, which should enable powerful new examples of combinatorial antigen recognition of cancer and other disease tissues.

Engineering Three-Input AND Gate Circuits Using Multiple synNotch Receptor Cascade

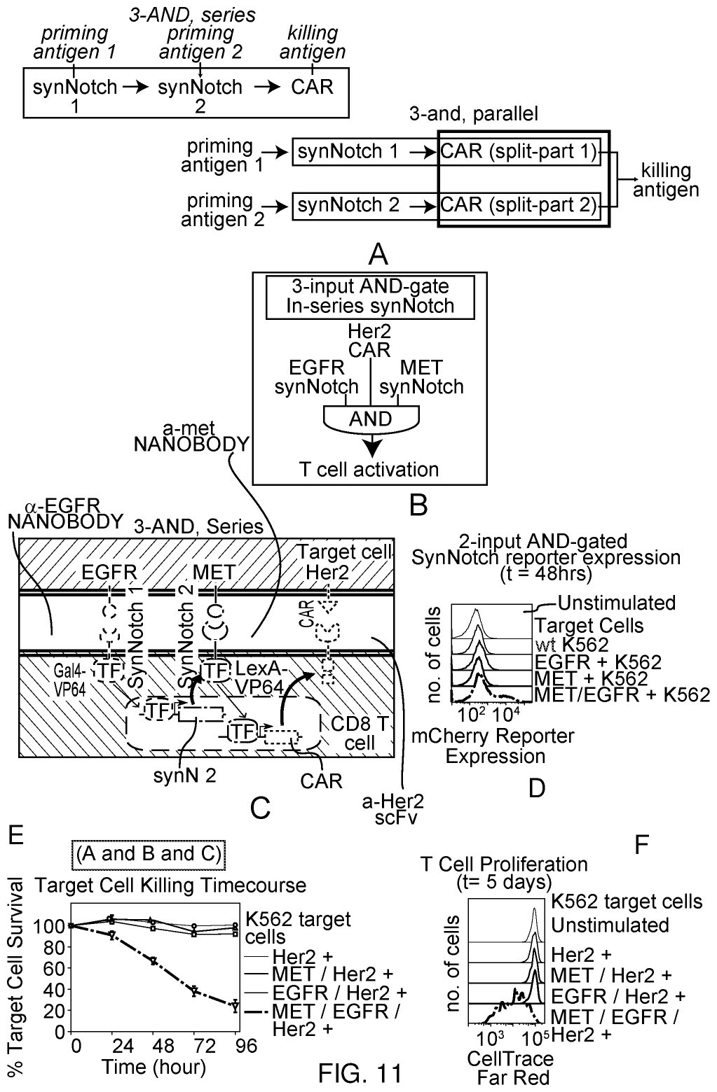

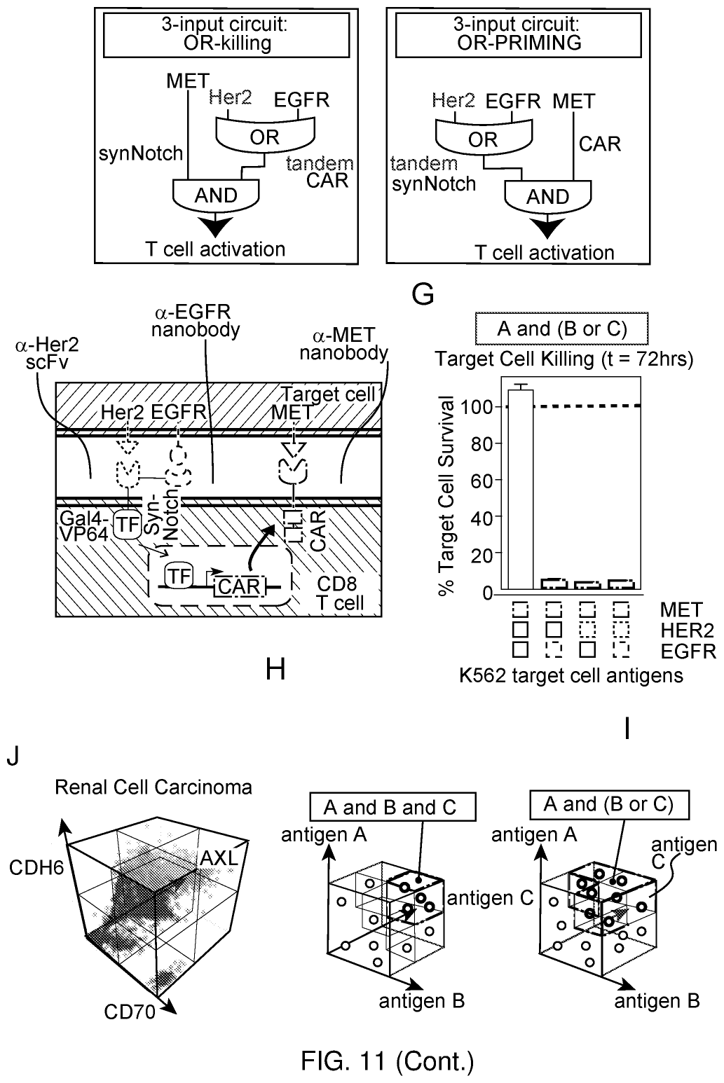

It was hypothesized that tumor recognition specificity could be even further improved if AND gate sensing could be extended from 2 to 3-inputs. To build a system requiring recognition of three independent antigen inputs to trigger T cell activation, the use of two orthogonal synNotch receptors in the same cell to control CAR expression was sought. There are two possible schemes to construct three input AND gates (FIG. 11A): 1) in-Series AND gate circuit in which one synNotch receptor controls expression of a second synNotch receptor, which in turn controls expression of a CAR; 2) in-Parallel AND gate circuit in which two synNotch receptors each control expression of components of a split CAR. In both of these schemes, each receptor recognizes a different antigen, leading to three antigen control.

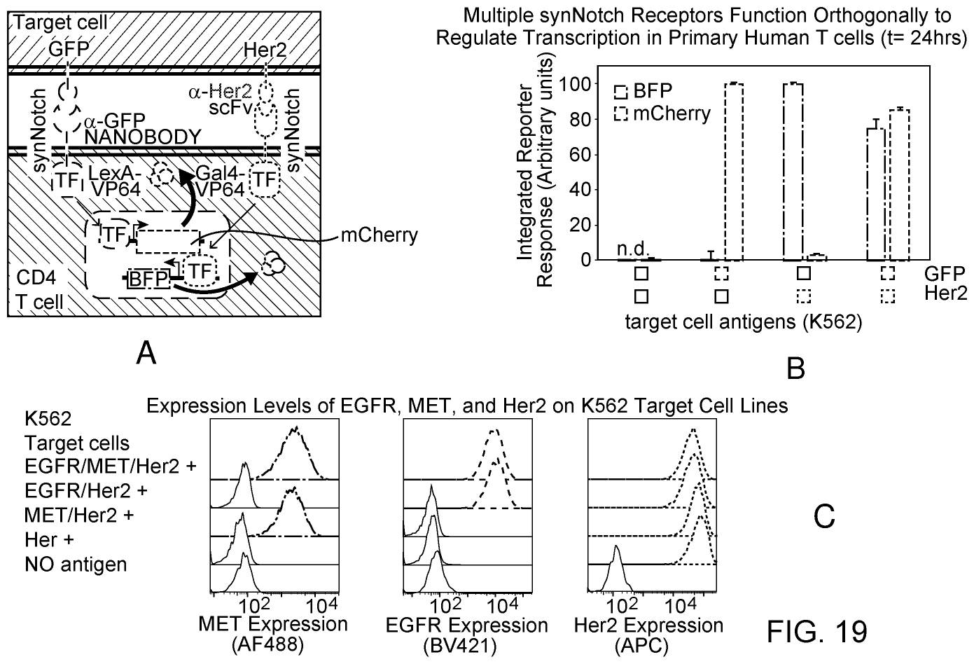

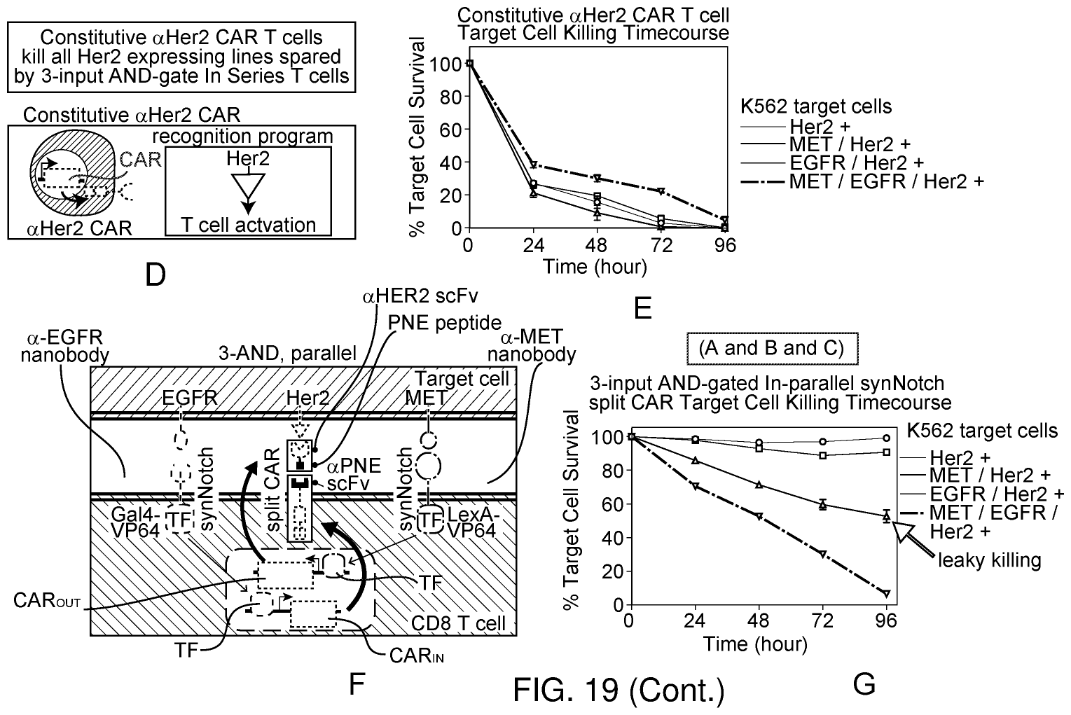

Executing design of such 3-input circuits involved first developing compatible synNotch platforms that utilize distinct intracellular transcriptional domains to regulate independent transcriptional programs. An α-Her2 Gal4-VP64 synNotch and an α-GFP LexA-VP64 synNotch, controlling expression of BFP (Gal4 UAS) and mCherry (LexA UAS) reporters, respectively (FIG. 19A) were engineered. Then human primary CD4+ T cells containing these double synNotch receptors were co-cultured with K562 target cells expressing GFP, Her2, GFP/Her2, or neither antigen. Reporter protein expression showed that the two synNotch receptors were able to generate robust independent responses with no crosstalk (FIG. 19B).

To prototype 3-input AND gates, the decision was made to target the combination of tumor antigens EGFR, MET, and HER2. For the in-Series AND gate, a circuit in which an α-EGFR synNotch receptor induced expression of an α-Met synNotch receptor, which in turn induced expression of an α-Her2 CAR, was designed. Here the two synNotch receptors use orthogonal transcriptional domains to avoid cross-talk. It was first shown that T cells engineered with an α-EGFR Gal4-VP64 synNotch in-Series with an α-MET LexA-VP64 controlling mCherry expression only activated the fluorescent reporter in response to EGFR+/MET+ K562 target cells, but not cells expressing either single antigen (FIG. 11D). Next expression of an α-Her2 CAR was put under control of a EGFR→MET in-Series synNotch circuit, and it was found that T cells engineered with this circuit selectively were activated by and killed MET+/EGFR+/Her2+ K562 target cells, while sparing cells expressing Her2 alone or with only one of the two other antigens in the circuit (FIGS. 11B,C,E,F; 19C). The highly specific killing by the 3-input in-series circuit T cells was in stark contrast to the indiscriminant killing of all Her2+ cells by constitutive α-Her2 CAR T cells (FIG. 19D).

An analogous in-Parallel 3 input AND gate was also constructed using the same antigen targets, but in this case the EGFR and MET synNotch receptors were used to drive expression of separate components of a split α-Her2 CAR (a peptide-specific CAR requires a Her2 scFv-peptide fusion as an adapter [Rodgers et al., 2016]). Testing of this in-Parallel circuit is shown in FIGS. 19F, 19G. This circuit enhanced specificity for target cells expressing all three target antigens but showed significantly increased leakiness compared to the in-Series circuit—moderate levels of killing were observed for cells expressing only two of three antigens. Thus, the three-step mechanism of the in-Series circuit appears to inherently lead to far more stringent control over activation, and thus appears to be a superior circuit design.

Overall, 3-input AND gates that take advantage of the modularity of synNotch receptors can be easily constructed. The in-Series scheme leads to the most stringent control of killing activity, while still maintaining effective killing. The ability to extend AND-gating from 2-antigen to 3-antigen sensing will enable engineered T cells to attack much more tightly defined antigen signatures, improving their potential discrimination between cancer and healthy cells.

Engineering 3-Input OR-AND Circuits that Balance Recognition Specificity and Flexibility to Mitigate Antigen Heterogeneity and Loss

While increased selectivity is important to avoid normal tissue cross-reactivity, overly stringent selectivity could be problematic in attacking tumors that show heterogeneous antigen expression or that show loss of antigen expression as a means of resistance. Prior work has shown that OR gate receptors—e.g. CAR's that have two different antigen recognition heads in tandem and can activate in response to either of two target antigens—can help significantly to overcome tumor heterogeneity and to minimize the probability of tumor resistance by target antigen loss (Fry et al., 2017; Grada et al., 2013; Hegde et al., 2016; Zah et al., 2016). Here integrating similar OR gate flexibility into the higher order synNotch→CAR/TCR circuits that have been engineered (FIG. 11G)is shown. It is obvious how to integrate OR-killing functionality by having a synNotch receptor induce expression of a tandem CAR with two recognition heads.

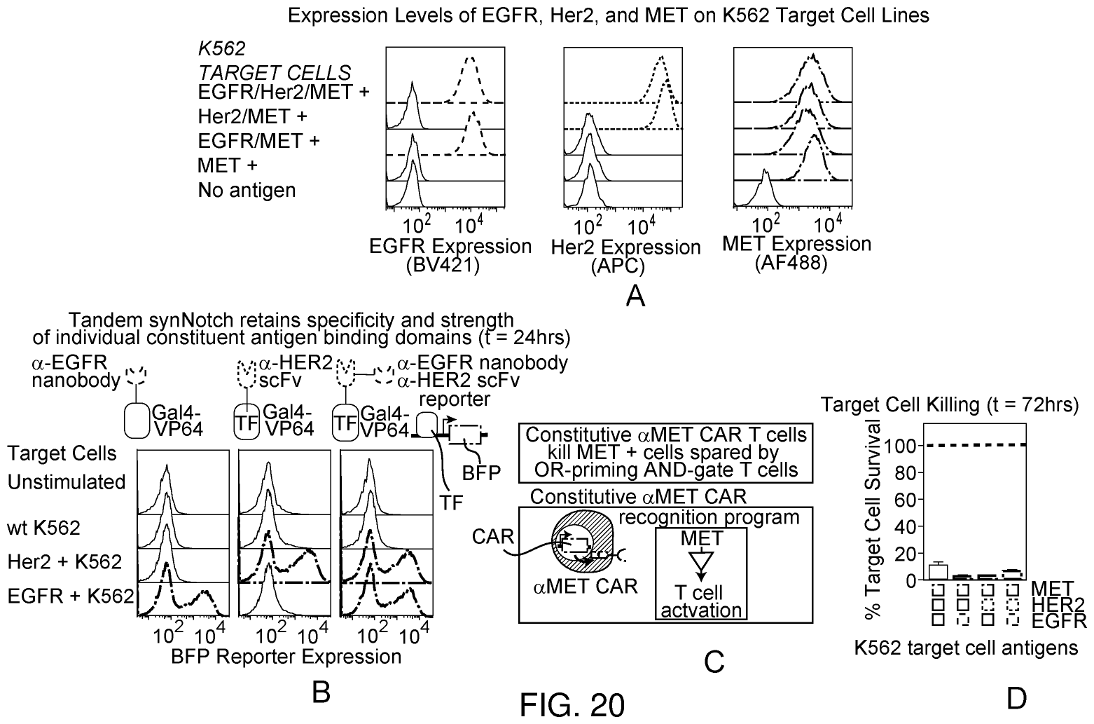

Here, the question was whether OR-priming circuits—where two different priming antigens could trigger the first step in activation of a synNotch→CAR circuit (circuit: if detect antigens A or B, then kill based on antigen C) could be constructed. Thus, a tandem synNotch that linked α-EGFR and α-Her2 binding domains with a flexible linker was engineered. It was found that T cells expressing this synNotch receptor robustly responded to either EGFR positive or Her2 positive K562 target cells, inducing expression of the BFP reporter at similar levels to that observed in T cells with either of the single antigen synNotch receptors (FIGS. 20A,B). Next, human primary CD8+ T cells with an α-EGFR/Her2 dual synNotch controlling expression of an α-MET CAR (FIG. 11H) were engineered. The 3-input OR→AND T cells only killed MET positive K562 target cells that also co-expressed EGFR or Her2, sparing MET only target cells in stark contrast to less specific killing by constitutive α-MET CAR T cells (FIGS. 11I, 20C,D).

Overall, modular receptors can be used to build 3-antigen recognition circuits that parse antigen space in different ways, as illustrated in the 3D antigen space diagrams in FIG. 11J. First, 3-input AND gates that require expression of all 3 antigens to induce activation/killing to achieve extremely high selectivity can be built (see example 3D antigen space plot in FIG. 11J of how the 3-antigen combination of CD70/CDH10/AXL is predicted to effectively segregate RCC cancer samples from normal tissue samples). But, second, 3-input circuits that incorporate OR flexibility on the side of the priming antigen(s) or the killing antigen(s) can also be built. Thus, if heterogeneity and escape through antigen loss are concerns, these types of circuits could be used.

### METHODS

### Source of Primary Human T Cells

Blood was obtained from Blood Centers of the Pacific (San Francisco, CA) as approved by the University Institutional Review Board. Primary CD4+ and CD8+ T cells were isolated from anonymous donor blood after apheresis (described in METHOD DETAILS).

### Defining the Space of Candidate Antigens

Potential candidate antigens were defined as genes with known or predicted cell surface expression, restricting the search space to current clinical targets and genes coding for transmembrane proteins. More specifically, a set of 29 unique clinical antigens along with their indications that have shown promise in the literature or are targets in currently active CAR or TCR trials were assembled and mapped to their corresponding genes. To assemble the list of transmembrane proteins the 3,305 protein coding genes annotated to the plasma membrane with high confidence (level 3 or higher) in the COMPARTMENTS database (Binder et al., 2014) were taken. The COMPARTMENTS database uses a combination of manually curated literature, text mining, high-throughput screens, and sequence prediction methods to make subcellular location predictions.

### Gene Expression Data Processing

Gene level RSEM processed TPM counts for healthy human tissue samples from the Genotype Tissue Expression (GTEx) project version 7 and gene level RSEM processed tumor samples from The Cancer Genome Atlas (TCGA) firehose were gathered. All together there were 21,486 samples covering 34 tissues and 33 cancer types (see tumor_and_tissue_counts.xlsx table for individual breakdowns per tissue and tumor type). To remove differences due to technical variation and thus combine these data from these two different sources, batch correction using a parametric empirical Bayes framework using the COMBAT function in the SVA R package (Johnson et al., 2007) was applied.

### Intelligent Subsampling and Data Partitioning

To increase the speed of the clustering score calculations as well as partition the data into training and test sets geometric sketching (Hie et al., 2019) was used. Geometric sketching allows us to subsample the space of samples maintaining the overall structure of the data by fitting a plaid covering and sampling points from within each region of the covering. In simulations across 8 different sketch sizes for 5 iterations across 100 gene pairs (10 fixed genes paired with 10 random genes) no loss of performance (see FIG. 15B) when calculating Davies-Bouldin and Manhattan distance were observed but substantial gains in runtime were observed. Based on these simulations a sketch size of 20% of all data was chosen for calculating clustering-based scores as well as the training data for classification and the remaining 80% of the data was held out for testing the classification models (FIG. 15B).

### Clustering-Based Scores

A method from the field clustering evaluations, Davies-Bouldin (DB), was utilized to measure the ratio of within cluster spread to cluster distance. The case where there are 2 clusters: a tumor cluster (given set of tumor samples) and a tissue cluster (all normal tissues samples) was considered. Lower DB scores are better as they indicate less within cluster distance (more tightly packed samples) and more distance between the cluster centers (more distance between tumor and normals).

$$DB = \frac{1}{N} {\sum }_{i = 1}^{N} {max}_{i \neq j} ( {R}_{i , j} ) ; {R}_{i , j} = \frac{{s}_{i} + {s}_{j}}{{M}_{i , j}} ; {S}_{i} = \frac{1}{{T}_{i}} {\sum }_{j = 1}^{{T}_{i}} \vert {X}_{j} - {A}_{i} \vert ; {M}_{i , j} = \vert {A}_{i} - {A}_{j} \vert$$

To give extra weight to distance the Manhattan distance between the normal and the tumor clusters was also calculated and this was used in the final clustering score. To compute a more interpretable clustering-based score to use throughout the search, log scaled DB and log scaled distance values across all tumor samples were calculated and the minimum of these two scores was taken with 1 representing the highest DB score and the largest scaled distance.

$${clustscore}_{t , {p}_{i , j}} = {min}_{i , j} ( \frac{{DB}_{t , {p}_{i , j}} - {min}_{x} ( {DB}_{t , x} )}{{max}_{x} ( {DB}_{t , x} ) - {min}_{x} ( {DB}_{t , x} )} , \frac{{d}_{t , {p}_{i , j}} - {min}_{x} ( {d}_{t , x} )}{{max}_{x} ( {d}_{t , x} ) - {min}_{x} ( {d}_{t , x} )} )$$

### Evaluation of Top Clustering-Based Scores

The top 10 antigen pairs per antigen class (C:C, C:N, and N:N) were chosen for each tumor based on their clustering scores for a total of ˜330 pairs per tumor type. Within the top 10 per class per tumor a particular gene in a pair was only allowed to appear a maximum of two times, preventing potential pairs from being dominated by a single gene with high separation. The analysis was further restricted to single antigens that are high, and pairs of antigens that have at least one antigen predicted to have high expression (high:high, high:low, and low:high) pairs.

To calculate the discriminatory ability of any particular antigen or antigen combination decision tree (DT) models on the 20% training partition were constructed using antigen expression as features and performance on the held out 80% of the data was evaluated. More explicitly, for antigen pairs the rpart R package was used to construct two single feature decision trees with c=−1 and a max depth=1 forcing each tree to have a single split. These splits were then used to draw a classification boundary and precision (the proportion of predicted positives that are correct), recall (the proportion of real positives that are predicted positive), and F₁ scores (the harmonic mean of precision and recall) were calculated, as shown in the following.

$${F}_{1} = \frac{precision \cdot recall}{precision + recall}$$

### Construct Design

All synNotch receptors used in this study were built using the mouse Notch1 (NM_008714) minimal regulatory region (Ile1427 to Arg 1752). The following binding domains were engineered into synNotch receptors: α-Axl scFv (as described in WO2012175691A1), α-Her2 scFv clone 4D5-8 (Liu et al., 2015), α-GFP nanobody clone LaG17 (Fridy et al., 2014), α-HLA-A2/AFP₁₅₈₋₁₆₆ scFv clone ET1402L1 (Liu et al., 2017), α-FAP scFv clone 28H1 (as described in WO2012020006A2) , α-MET nanobody clone 6C12 (as described in WO2012042026A1) , α-EGFR nanobody clone 9G8 (Roovers et al., 2011), α-CDH6 scFv clone V10 (as described WO2016024195A1) , and the CD27 extracellular domain. To build the tandem synNotch, the α-EGFR nanobody clone 9G8 was linked with a G4S linker to the N-terminus of an α-Her2 scFv clone 4D5-8 synNotch receptor. synNotch receptors were designed to include either Gal4 DNA-binding domain (DBD) VP64 or LexA DBD VP64 fusion proteins as a synthetic transcription factor. All synNotch receptors contain an N-terminal CD8α signal peptide for membrane targeting. Following the CD8α signal peptide, Gal4 synNotch receptors contain a myc tag and LexA synNotch receptors contain a FLAG tag for easy and orthogonal surface detection with α-myc AF647 (Cell Signaling #2233) and α-FLAG AF488 (R&D Systems #IC8529G), respectively.

All CARs used in this study were designed by fusing scFvs to the human CD8α chain hinge and transmembrane domains and the cytoplasmic regions of the human 4-1BB and CD3ζ signaling proteins. The following binding domains were engineered into CARs: α-Axl scFv (as described in WO2012175691A1), α-Her2 scFv clone 4D5-8 (Liu et al., 2015), α-Mesothelin nanobody clone G3A (as described in US20170267755A1) , α-MET nanobody clone 6C12 (as described in WO2012042026A1), and the CD27 extracellular domain. All CARs included an N-terminal myc tag or V5 tag for easy detection with α-myc AF647 (Cell Signaling #2233) or α-V5 PE (Thermo Fisher #12-6796-42). All CARs contain an N-terminal CD8α signal peptide.

Plasmids to express TCRs used in this study were constructed in a multicistronic configuration with T2A peptides enabling 1:1 stoichiometric expression of the TCR chains in a β-T2A-α configuration. The sequences of the following TCRs were used: α-HLA-A2/NY-ESO1 TCR 1G4 α95LY clone (Robbins et al., 2008) and α-HLA-A2/MART1 TCR DMF5 β54A clone (Robbins et al., 2008).

For experiments with T cells expressing a single synNotch receptor the Gal4 system was utilized, and the receptors were cloned into a modified pHR′SIN:CSW vector containing a constitutive SFFV or PGK promoter. For these experiments, the pHR′SIN:CSW vector was also modified to make the response element plasmids. Five copies of the Gal4 DNA binding domain target sequence were cloned 5′ to a minimal CMV promoter. Also included in the response element plasmids is a PGK promoter that constitutively drives mCherry or BFP expression to easily identify transduced T cells. For all synNotch response element vectors, the inducible transgene (e.x. CAR or TCR) was cloned via a BamHI site in the multiple cloning site 3′ to the Gal4 response elements. All constructs were cloned via InFusion Cloning (Takara Bio #638910).

For experiments with T cells expressing two synNotch receptors and some experiments with T cells expressing a single synNotch, single vector constructs were designed to contain a synNotch receptor and its corresponding response element. For these experiments, the pHR′SIN:CSW vector was modified to include both a constitutively expressed synNotch receptor as well as the corresponding response element. The Gal4 response element was designed as described above. For the LexA response element, 7 copies of the LexA DNA binding domain target sequence were cloned 5′ to a minimal CMV promoter. The sequence for the LexA repeat binding motif was obtained from (Ottoz et al., 2014) . The inducible transgene (e.x. CAR or TCR) was cloned via a BamHI site in the multiple cloning site 3′ to the Gal4 or LexA response elements. The constitutively expressed synNotch receptor was expressed from a PGK constitutive promoter 3′ to the inducible transgene. synNotch receptors were cloned via a NdeI site in the multiple cloning site 3′ PGK promoter. All constructs were cloned via InFusion Cloning (Takara Bio #638910).

### Primary Human T Cell Isolation and Culture

Primary CD4+ and CD8+ T cells were isolated from anonymous donor blood after apheresis by negative selection (STEMCELL Technologies #15062 and 15023). T cells were cryopreserved in RPMI-1640 (Corning #10-040-CV) with 20% human AB serum (Valley Biomedical, #HP1022) and 5% DMSO (Sigma-Aldrich #472301). After thawing, T cells were cultured in human T cell medium consisting of X-VIVO 15 (Lonza #04-418Q), 5% Human AB serum and 10 mM neutralized N-acetyl L-Cysteine (Sigma-Aldrich #A9165) supplemented with 30 units/mL IL-2 (NCI BRB Preclinical Repository) for all experiments.

### Lentiviral Transduction of Human T Cells and Target Cells

Lenti-X 293T packaging cells (Clontech #11131D) were cultured in medium consisting of Dulbecco's Modified Eagle Medium (DMEM) (Gibco #10569-010), 10% fetal bovine serum (FBS) (Univeristy of California, San Francisco [UCSF] Cell Culture Facility), and gentamicin (UCSF Cell Culture Facility). Fresh packaging cells were thawed after cultured cells reached passage 30.

Pantropic VSV-G pseudotyped lentivirus was produced via transfection of Lenti-X 293T cells with a pHR′SIN:CSW transgene expression vector and the viral packaging plasmids pCMVdR8.91 and pMD2.G using Fugene HD (Promega #E2312). Primary T cells were thawed the same day, and after 24 hr in culture, were stimulated with Dynabeads Human T-Activator CD3/CD28 (Thermo Scientific #11131D) at a 1:3 cell:bead ratio. At 48 hr, viral supernatant was harvested and the primary T cells were exposed to the virus for 24 hr. At day 5 post T cell stimulation, Dynabeads were removed and the T cells expanded until day 12 when they were rested and could be used in assays. T cells were sorted for assays with a FACs ARIA II on day 5 or 6 post T cell stimulation.

K562s and T2 target cell lines were transduced to expressed all external antigens using lentivirus. All external antigens were expressed as fusion proteins of their extracellular domains fused to the PDGFR transmembrane domain (for the case of GFP, the entire GFP was fusion to the PDGFR transmembrane domain). Within the exception of GFP whose fluorescence was directly observed, antigen levels were determined via flow cytometry after staining cells with the following antibodies: α-Her2 APC (Biolegend #324408), α-EGFR BV421 (BD Biosciences #566254), and α-MET AF488 (R&D Systems #FAB3582G). All K562 and T2 cell lines were sorted via FACS for expression of the antigen transgenes. Cells transduced with different combinations of antigens were sorted with a FACS ARIA II for consistent expression levels of each individual antigen across different target cell lines.

### Cancer and Target Cell Lines

The cancer cell lines used were Raji B cell Burkitt lymphoma cells (ATCC #CCL-86), 769-P renal cell carcinoma cells (ATCC #CRL-1933), PANC04.03 pancreatic cancer cells (ATCC #CRL-2555), M262 melanoma cells (Søndergaard et al., 2010), M202 melanoma cells (Søndergaard et al., 2010) , K562 myelogenous leukemia cells (ATCC #CCL-243), and T2 lymphoblastoid cells (ATCC #CCL-1992). Rajis and 769-Ps were cultured in RPMI 1640 with L-glutamine (Corning #10-040-CV) supplemented with 10% FBS. PANC04.03s were cultured in RPMI 1640 with L-gluatmine, 15% FBS, and 20 Units/mL human recombinant insulin. M262 and M202 melanoma cells were cultured in RPMI 1640 with L-glutamine, 10% FBS, and 1% penicillin/streptomycin. K562s were cultured in medium consisting of Iscove's DMEM (Corning #10-016-CV), 10% FBS, and gentamicin. T2s were cultured in medium consisting of Iscove's DMEM supplemented with 20% FBS.

The immortalized healthy tissue cell lines or primary human cells were Beas2B lung epithelial cells (ATCC #CRL-9609), immortalized human pancreatic stellate cells (Hwang et al., 2008), and primary human epidermal melanocytes from lightly pigmented adult skin (Cascade Biologics #C-024-5C). Beas2B cells were cultured in BEBM (Lonza #CC3171) supplemented with the BEGM kit (Lonza #CC-3170). The human pancreatic stellate cells were cultured in DMEM supplement with GlutaMAX (Thermo Fisher #10569-044), 15% FBS, and 1% penicillin/streptomycin. The human epidermal melanocytes were cultured in Medium 254 (Cascade Biologics #M-254-500) supplemented with PMA-Free Human Melanocyte Growth Supplement-2 (Cascade Biologics #S-016-5).

For all cells other than K562 and T2 cell lines, levels of various antigens were determined via flow cytometry after staining cells with the following antibodies: α-CD70 (Biolegend #355109), α-Axl APC (R&D systems #FAB154A), α-CDH6 AF647 (R&D systems #FAB2715R), α-FAP AF647 (R&D systems #FAB3715R), α-Mesothelin APC (R&D systems #FAB32652A), α-MET AF488 (R&D Systems #FAB3582G), α-MART1 AF647 (Novus Biologicals #NBP2-47803AF647), and α-HLA-A2 APC (Biolegand #343308).

### Melanoma Cell Line RNA-Sequencing

RNA extraction was performed using the AllPrep DNA/RNA Mini kit (Qiagen #80204) for 53 human melanoma cell lines. Libraries were prepared using the Illumina TruSeq RNA (Illumina #RS-122-2001) sample preparation kit per the manufacturer's instructions. RNA sequencing was performed using 50-bp paired-end sequencing on the Illumina HiSeq 2000 platform. Reads were mapped using HISAT2 to the Homo sapiens hg38 genome build and raw counts were quantified using HTSeq. Raw counts were normalized to TPM values by dividing the counts by the library size scaling factor (total reads divided by one million) and the length of each gene in kilobases as defined by the union of all exons.

### Peptides

All antigen peptides used in experiments with T2 cells this study are HLA-A2-restricted. Synthetic peptides corresponding to cancer antigens were obtained from one of two sources. Peptides for AFP₁₅₈₋₁₆₆ and WT1₁₂₆₋₁₃₄ were custom synthesized by GenScript. The NY-ESO1₁₅₇₋₁₆₅ peptide (AnaSpec #AS-63823) was ordered from AnaSpec. Purity of peptides was verified at >98% by mass spectrometry performed by peptide supplier. Upon delivery, peptides were dissolved in dimethylsulfoxide (DMSO) at 2 mM and stored at −20° C. until use. For assays, peptides were thawed and serially-diluted in T2 cell medium.

### T2 Cell Peptide Pulsing

One to two million T2 cells were pulsed at a concentration of 10 million cells/mL in T2 cell media. T2 cells were pulsed with each peptide for 2 hours at 37° C. in wells of a 96-well round bottom plate. T2 cells were then diluted in T cell medium to the desired cell concentration for use in co-culture T cell assays. Unless indicated otherwise, T2s were pulsed with inNotch target antigen peptides at 10 μM and TCR target antigen peptides at 10 nM.

### Antibody Staining and Flow Cytometry Analysis

All antibody staining for flow cytometry was carried out in wells of round-bottom 96-well tissue culture plates. For co-culture assays performed in flat-bottom 96-well plates, all cells were lifted with TrypLE Express (Thermo Fisher #12604013) and transferred to a round-bottom 96-well plate for staining. Cells were pelleted by centrifugation of plates for 4 min at 400×g. Supernatant was removed and cells were resuspended in 50 μL PBS containing the fluorescent antibody of interest. Cells stained 25 minutes at 4° C. in the dark. Stained cells were then washed two times with PBS and resuspended in fresh PBS supplemented with 1% FBS and EDTA for flow cytometry with a BD LSR II. All flow cytometry data analysis was performed in FlowJo software (TreeStar).

In Vitro Stimulation of synNotch T Cells

For all in vitro synNotch T cell assays (including both reporter and killing assays), T cells were co-cultured with target cells at a 1:1 ratio, with anywhere from 1e4-1e5 each/well. The Countess II Cell Counter (ThermoFisher) was used to determine cell counts for all assay set up. T cells and target cells were mixed in 96-well tissue culture plates in 200 μL T cell media, and then plates were centrifuged for 1 min at 400×g to initiate interaction of the cells. For assays with Raji, K562, or T2 target cells, round-bottom 96-well plates were used. For assays with all other target cells, flat-bottom 96-well plates were used. Cells were co-cultured for anywhere from 18 to 96 hours before analysis via flow cytometry with a BD LSR II.

### Flow Cytometry-Based T Cell Cytotoxicity Assay

For all cytotoxicity assays, synNotch T cells, constitutive CAR or TCR T cells, or untransduced T cells were co-cultured with target cells at a 1:1 ratio as described above. After intended periods of incubation, samples were centrifuged for 4 min at 400×g, after first being transferred to a round-bottom 96-well plate if necessary. Supernatant were then removed and cells were resuspended in PBS supplemented with 1% FBS and EDTA for flow cytometry with a BD LSR II. Control samples containing only the target cells were used to set up flow cytometry gates for live target cells based on forward and side scatter patterns. For assays with K562 target cells, target cells were further gated on high mCherry fluorescence, as all K562 lines used in assays were engineered to express a cytoplasmic mCherry marker. For assays with T2 target cells, targets cells were gated on low CellTrace Violet dye (ThermoFisher #C34557) fluorescence, as T cells used in these assays were labeled with CellTrace Violet to enable clear differentiation of populations. For assays with all other target cells, target cells were gated on low CellTrace Far Red dye (Thermo Fisher #C34564) or low CD3 staining, as T cells used in these assays were either labeled with CellTrace Far Red or the samples were stained with □-CD3 APC/Cy7 (Biolegend #317342) to specifically label T cells. The level of target cell survival was determined by comparing the fraction of target cells alive in the culture compared to treatment with untransduced T cell controls.

### Trans-Killing K562 Target Cell Killing Assay

5e4 T cells were co-cultured with 5e4 K562 cells in various ratios as described in the figure in complete human T cell media. After mixing the T cells and cancer cells in round bottom 96-well tissue culture plates, the cells were centrifuged for 1 min at 400×g to force interaction of the cells, and the cultures were analyzed at 24-72 hr for activation and specific lysis of target tumor cells. Control samples containing only the target cells were used to set up flow cytometry gates for live target cells based on forward and side scatter patterns. All K562 cells were stained with CellTrace Far Red. To distinguish the priming cells from target cells, the target cells were stained additionally with CellTrace CSFE (Thermo Fisher #C34554) . T cells used in the assay were labeled with CellTrace Violet. The level of target cell survival was determined by comparing the fraction of target cells alive in the culture compared to treatment with untransduced T cell controls. All flow cytometry was performed using Attune N×T Flow Cytometer and the analysis was performed in FlowJo software (TreeStar).

### IncuCyte-Based T Cell Trans-Killing Cytotoxicity Assay

2,5e4 GFP+ target cells (Panc04.03) and 2.5e4 mCherry+ priming cells (FAP+ pancreatic stellate cells or FAP− 3T3 cells) were plated in a 96 well flat bottom plate overnight. 5.0e4 T Cells were then added to each well and total GFP and mCherry signal from each well was measured every 2 hours using an Incucyte Live cell Imaging system (Essen Biosciences).

### IFNγ ELISA

1e4 T cells expressing an α-MART-1 TCR constitutively or under control of an α-MET synNotch were co-cultured at 1:1 with HLA-A2+ primary human epidermal melanocytes in a flat-bottom 96-well plate. After 24 hours, supernatant was harvested from T cells that had either been stimulated with the melanocytes or left unstimulated in a well alone. Harvested supernatants were stored at −80° C. until analysis. IFN-γ levels in the supernatant were determined via an IFN-γ ELISA (Thermo Fisher #EHIFNG2).

### Primary Human Melanocyte Microscopy Killing Assay

2e4 primary human epidermal melanocytes were labelled with CellTrace Far Red and plated in wells of a flat-bottom 96-well plate and allowed to adhere. After 24 hours, 2e4 primary human CD8+ UnT, constitutive α-MART-1 TCR, or α-MET synNotch/α-MART-1 TCR were added to the wells with melanocytes. Microscopy images were taken shortly after melanocyte plate and then 96 hours after T cell plating. All imaging was performed on an Opera Phenix High-Content Screening System (PerkinElmer) with a 20× water dipping lens. Briefly, images were taken in flat-bottom tissue culture plastic 96-well plates with 12 slices at 1 μm spacing and 3×3 tiling. For analysis, each image was produced with max z projections per well.

### Tracking of T Cell Proliferation

Primary human CD8+ synNotch or constitutive TCR T cells were labeled with CellTrace Far Red following manufacturer's instructions. T cells were co-cultured with target cells at a 1:1 ratio as described above. After 4 or 5 days, samples were centrifuged for 4 min at 400×g prior to resuspension in fresh PBS supplemented with 1% FBS and EDTA for flow cytometry with a BD LSR II. For flow cytometry analysis, T cells were specifically distinguished by FSC and SSC pattern and high levels of CellTrace Far Red fluorescence, as even highly proliferating T cells had stronger fluorescence than the target cells.

This analysis described above provides a roadmap for a precision medicine that more deeply integrates in silico data analysis with capabilities emerging from synthetic biology and cell design. In this case, large-scale genomic data may be used to stratify patients based on likelihood of response to a drug, but rather the data becomes the guide for how to best design a smart cellular drug. Here, opportunities to discover, within the multidimensional space of antigens, the signatures that can offer the optimal recognition discrimination can be identified.

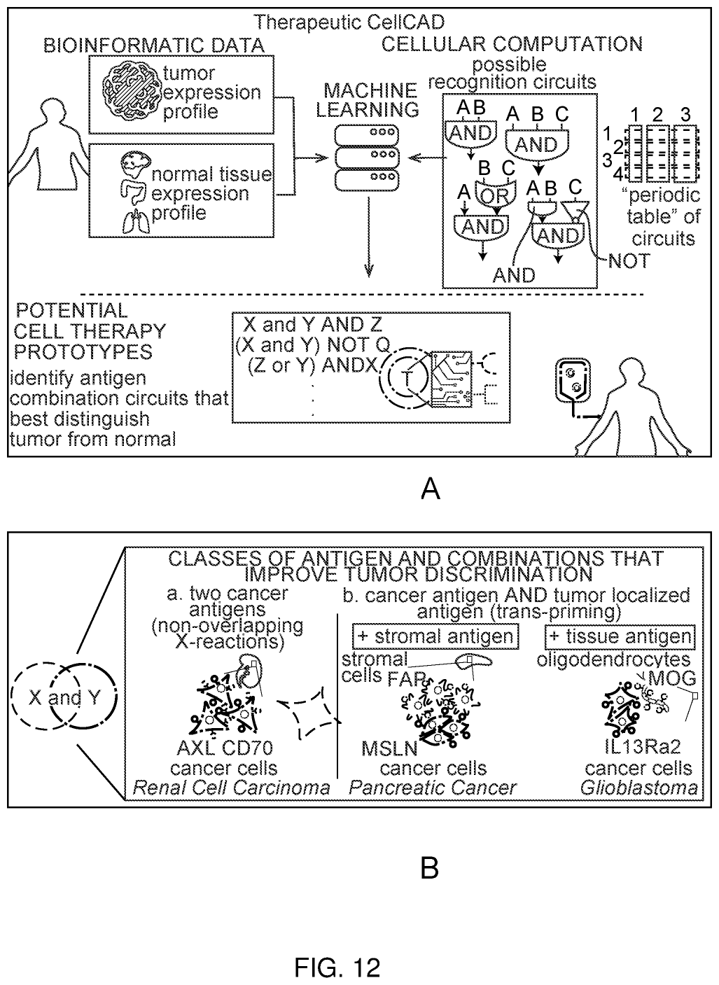

An exemplary scheme of how this combinatorial information might be used is shown in FIG. 12A. Computer aided cell design would use machine learning algorithms to match discriminatory antigen combinations identified in silico with the sets of validated recognition circuits available in the synthetic biology toolkit. Here, the filtering and validation of what types of recognition circuits and targets are ideal with respect to minimal cross-reaction and maximal capture of the cancer are shown, as well as which combinations are actionable (i.e. can be built from existing modular components).

### Targeting Antigen Combinations Can Significantly Improve Tumor Recognition

The in silico analysis, based on available RNAseq expression datasets, predicts that using Boolean antigen combinations can significantly improve the selectivity of tumor recognition and avoidance of normal tissue cross-reactivity. Thus, using Boolean multi-antigen detecting engineered T cells has the potential to have a major impact on cancer recognition and the development of next generation cellular therapies.

Most concretely, it has been found that adding new antigens to current clinical antigen targets (CAR or TCR) via AND or NOT Boolean recognition is predicted to significantly increase cancer versus normal tissue discrimination. Moreover, many novel:novel antigen pairs that show even stronger and closer to ideal discrimination can be found. Every cancer type examined here has more than 25 antigen pairs that are predicted to show discrimination, with clustering-based scores above 0.85 (out of ideal 1.0). Thus, there are likely to be many options of multi-antigen signatures that could be used to recognize any one type of tumor. Data for possible antigen pairs for all cancer types analyzed will be made accessible at a searchable interactive webserver.

### Information-Rich Antigen Pairs: Harnessing Complementary Cancer Antigens or Incorporating Microenvironmental Antigens

Several general patterns emerge from the analysis concerning what types of antigen combinations can provide significantly enhanced tumor discrimination (FIG. 12B). First, is a scenario in which two cancer antigens expressed in the same tumor show distinct and therefore complementary cross-reactions. An example of this scenario is the recognition of AXL and CD70 in renal cell carcinoma (RCC). The cross-reactions of CD70 are largely with hematopoetic lineages, while the cross-reactions of AXL are with epithelial tissues such as lung cells. Thus, requiring both antigens for therapeutic recognition (AND gate) eliminates both classes of cross-reaction.

A second general pattern of enhanced discrimination comes from utilizing an antigen on the cancer cells combined with an antigen from other distinct non-cancer cells in the tumor microenvironment. The second antigen could be on cancer-associated stromal cells, as is the case for the characteristic high expression of FAP on the stromal cells of pancreatic tumors (including metastases). Alternatively, the second antigen could be a tissue specific antigen that is ubiquitously expressed in the location of the tumor. An example of this is the enhanced recognition of glioblastoma by combining the cancer antigen IL13Ra2 with the brain specific antigen MOG (expressed on oligodendrocytes of the myelin sheath), shown in FIG. 7G. These examples that integrate recognition of antigens on two different cell populations within the tumor depend on the ability of synNotch→CAR prime-and-kill circuits to mediate trans-antigen recognition (priming off of one cell and killing of another neighboring cell).

### Enumerating a “Periodic Table” of Possible Cell-Based Recognition Circuits

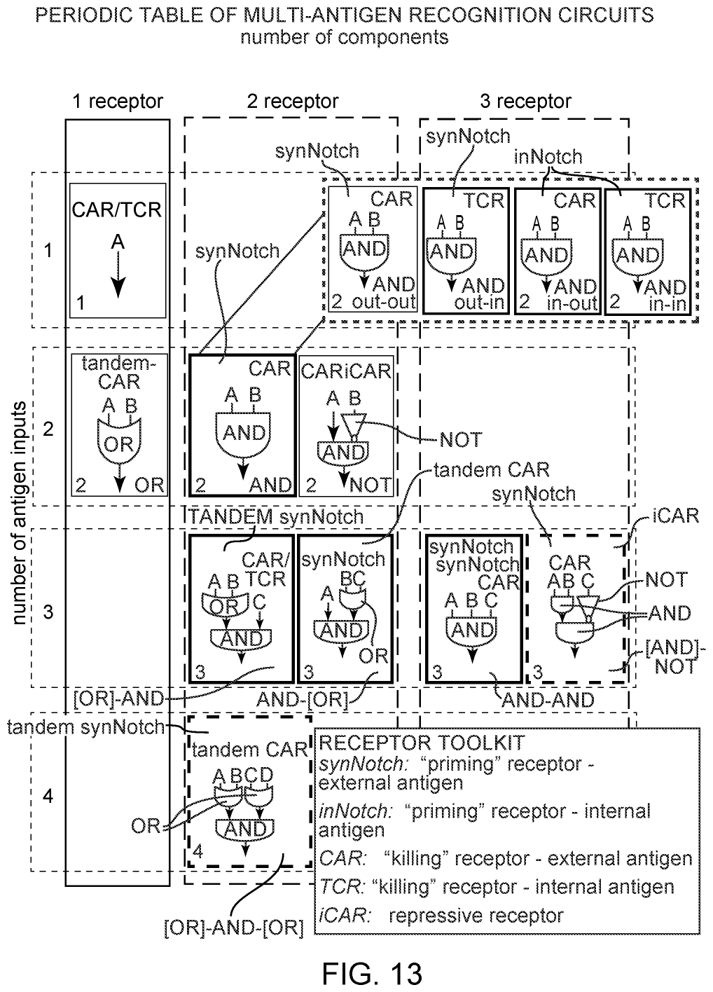

Another key demonstration in this manuscript is that the toolbox of modular receptors can be used to engineer a wide array of different multi-antigen recognition circuits. FIG. 13 shows a current state-of-the-art “periodic” table of the potential recognition circuits that can now be constructed using modular sensors such as CARs, TCRs, synNotch receptors, and inhibitory CARs (iCARs). The table is organized by both number of receptor components used to construct the circuit, as well as the number of antigen inputs the circuit integrates for recognition. The table enumerates the space of possible circuits that detect up to 3 antigens. Prototypes of the majority of these possible recognition circuits have been able to be constructed. Although a few circuits types have not been validated, their design is straightforward and their likelihood of functioning high, given other analogous related circuits that have been tested. As this field grows, there will likely emerge alternative mechanisms for building these recognition circuits, but the overall categories of recognition circuit functions is unlikely to change significantly.

The range of recognition functions that can be achieved will likely have a major impact on how engineered cell therapies can detect cancer and other diseases. The variety of recognition modalities means that there is great potential to sector multi-dimensional antigen space in a diversity of ways to find those ways that best segregate disease tissues from normal tissues. Thus, harnessing the computational capabilities of living cells, and using in silico analysis to guide their deployment, provides a broad new frontier for recognizing and attacking complex diseases such as cancer.

### REFERENCES

Aguet, F., Brown, A. A., Castel, S. E., Davis, J. R., He, Y., Jo, B., Mohammadi, P., Park, Y., Parsana, P., Segrè, A. V., et al. (2017). Genetic effects on gene expression across human tissues. Nature 550, 204-213.

Almén, M., Nordström, K. J., Fredriksson, R., and Schiöth, H. B. (2009). Mapping the human membrane proteome: a majority of the human membrane proteins can be classified according to function and evolutionary origin. Bmc Biol 7, 50.

Beatty, G. L., Haas, A. R., Maus, M. V., Torigian, D. A., Soulen, M. C., Plesa, G., Chew, A., Zhao, Y., Levine, B. L., Albelda, S. M., et al. (2014). Mesothelin-Specific Chimeric Antigen Receptor mRNA-Engineered T Cells Induce Antitumor Activity in Solid Malignancies. Cancer Immunol Res 2, 112-120.

Binder, J. X., Pletscher-Frankild, S., Tsafou, K., Stolte, C., O'Donoghue, S. I., Schneider, R., and Jensen, L. (2014). COMPARTMENTS: unification and visualization of protein subcellular localization evidence. Database 2014, bau012.

Chodon, T., Comin-Anduix, B., Chmielowski, B., Koya, R. C., Wu, Z., Auerbach, M., Ng, C., Avramis, E., Seja, E., Villanueva, A., et al. (2014). Adoptive Transfer of MART-1 T-Cell Receptor Transgenic Lymphocytes and Dendritic Cell Vaccination in Patients with Metastatic Melanoma. Am Assoc Cancer Res 20, 2457-2465.

Cohen, A. D., Garfall, A. L., Stadtmauer, E. A., Melenhorst, J. J., Lacey, S. F., Lancaster, E., Vogl, D. T., Weiss, B. M., Dengel, K., Nelson, A., et al. (2019). B cell maturation antigen-specific CAR T cells are clinically active in multiple myeloma. J Clin Invest.

de Cristofaro, T., Palma, T., Soriano, A., Monticelli, A., Affinito, O., Cocozza, S., and Zannini, M. (2016). Candidate genes and pathways downstream of PAX8 involved in ovarian high-grade serous carcinoma. Oncotarget 7, 41929-41947.

Fedorov, V. D., Themeli, M., and Sadelain, M. (2013). PD-1- and CTLA-4-Based Inhibitory Chimeric Antigen Receptors (iCARs) Divert Off-Target Immunotherapy Responses. Sci Transl Med 5, 215ra172-215ra172.

Fry, T. J., Shah, N. N., Orentas, R. J., Stetler-Stevenson, M., Yuan, C. M., Ramakrishna, S., Wolters, P., Martin, S., Delbrook, C., Yates, B., et al. (2017). CD22-targeted CAR T cells induce remission in B-ALL that is naive or resistant to CD19-targeted CAR immunotherapy. Nat Med 24, 20.

Grada, Z., Hegde, M., Byrd, T., Shaffer, D. R., Ghazi, A., Brawley, V. S., Corder, A., Schönfeld, K., Koch, J., Dotti, G., et al. (2013). TanCAR: A Novel Bispecific Chimeric Antigen Receptor for Cancer Immunotherapy. Mol Ther—Nucleic Acids 2, e105.

Hegde, M., Corder, A., Chow, K. K., Mukherjee, M., Ashoori, A., Kew, Y., Zhang, Y., Baskin, D. S., Merchant, F. A., Brawley, V. S., et al. (2013). Combinational Targeting Offsets Antigen Escape and Enhances Effector Functions of Adoptively Transferred T Cells in Glioblastoma. Mol Ther 21, 2087-2101.

Hegde, M., Mukherjee, M., Grada, Z., Pignata, A., Landi, D., Navai, S. A., Wakefield, A., Fousek, K., Bielamowicz, K., Chow, K., et al. (2016). Tandem CAR T cells targeting HER2 and IL13Rα2 mitigate tumor antigen escape. J Clin Invest 126, 3036-3052.

Hie, B., Cho, H., DeMeo, B., Bryson, B., and Berger, B. (2019). Geometric Sketching Compactly Summarizes the Single-Cell Transcriptomic Landscape. Cell Syst. 6, 483-493.

Hintzen, R. Q., Lens, S. M., Koopman, G., Pals, S. T., Spits, H., and van Lier, R. A. (1994). CD70 represents the human ligand for CD27. Int Immunol 6, 477-480.

Hwang, R. F., Moore, T., Arumugam, T., Ramachandran, V., Amos, K. D., Rivera, A., Ji, B., Evans, D. B., and Logsdon, C. D. (2008). Cancer-Associated Stromal Fibroblasts Promote Pancreatic Tumor Progression. Cancer Res 68, 918-926.

Jilaveanu, L. B., Sznol, J., Aziz, S. A., Duchen, D., Kluger, H. M., and Camp, R. L. (2012). CD70 expression patterns in renal cell carcinoma. Hum Pathol 43, 1394-1399.

Johnson, E. W., Li, C., and Rabinovic, A. (2007). Adjusting batch effects in microarray expression data using empirical Bayes methods. Biostatistics 8, 118-127.

Johnson, L. A., Heemskerk, B., Powell, D. J., Cohen, C. J., Morgan, R. A., Dudley, M. E., Robbins, P. F., and Rosenberg, S. A. (2006). Gene Transfer of Tumor-Reactive TCR Confers Both High Avidity and Tumor Reactivity to Nonreactive Peripheral Blood Mononuclear Cells and Tumor-Infiltrating Lymphocytes. J Immunol 177, 6548-6559.

Johnson, L. A., Morgan, R. A., Dudley, M. E., Cassard, L., Yang, J. C., Hughes, M. S., Kammula, U. S., Royal, R. E., Sherry, R. M., Wunderlich, J. R., et al. (2009). Gene therapy with human and mouse T-cell receptors mediates cancer regression and targets normal tissues expressing cognate antigen. Blood 114, 535-546.

Kloss, C. C., Condomines, M., Cartellieri, M., Bachmann, M., and Sadelain, M. (2013). Combinatorial antigen recognition with balanced signaling promotes selective tumor eradication by engineered T cells. Nat Biotechnol 31, 71.

Kraman, M., Bambrough, P., Arnold, J., Roberts, E., Magiera, L., Jones, J., Gopinathan, A., Tuveson, D., and Fearon, D. (2010). Suppression of Antitumor Immunity by Stromal Cells Expressing Fibroblast Activation Protein-. Science 330, 827-830.

Lamers, C. H., Sleijfer, S., van Steenbergen, S., van Elzakker, P., van Krimpen, B., Groot, C., Vulto, A., den Bakker, M., Oosterwijk, E., Debets, R., et al. (2013). Treatment of Metastatic Renal Cell Carcinoma With CAIX CAR-engineered T cells: Clinical Evaluation and Management of On-target Toxicity. Mol Ther 21, 904-912.

Liu, H., Xu, Y., Xiang, J., Long, L., Green, S., Yang, Z., Zimdahl, B., Lu, J., Cheng, N., Horan, L. H., et al. (2017). Targeting Alpha-Fetoprotein (AFP)—MHC Complex with CAR T-Cell Therapy for Liver Cancer. Clin Cancer Res 23, 478-488.

Liu, X., Jiang, S., Fang, C., Yang, S., Olalere, D., Pequignot, E. C., Cogdill, A. P., Li, N., Ramones, M., Granda, B., et al. (2015). Affinity-Tuned ErbB2 or EGFR Chimeric Antigen Receptor T Cells Exhibit an Increased Therapeutic Index against Tumors in Mice. Cancer Res 75, 3596-3607.

Maude, S. L., Laetsch, T. W., Buechner, J., Rives, S., Boyer, M., Bittencourt, H., Bader, P., Verneris, M. R., Stefanski, H. E., Myers, G. D., et al. (2018). Tisagenlecleucel in Children and Young Adults with B-Cell Lymphoblastic Leukemia. New Engl J Medicine 378, 439-448.

Mbalaviele, G., Nishimura, R., Myoi, A., Niewolna, M., Reddy, S. V., Chen, D., Feng, J., Roodman, D., Mundy, G. R., and Yoneda, T. (1998). Cadherin-6 Mediates the Heterotypic Interactions between the Hemopoietic Osteoclast Cell Lineage and Stromal Cells in a Murine Model of Osteoclast Differentiation. J Cell Biology 141, 1467-1476.

Morgan, R. A., Dudley, M. E., Wunderlich, J. R., Hughes, M. S., Yang, J. C., Sherry, R. M., Royal, R. E., Topalian, S. L., Kammula, U. S., Restifo, N. P., et al. (2006). Cancer Regression in Patients After Transfer of Genetically Engineered Lymphocytes. Science 314, 126-129.

Morgan, R. A., Yang, J. C., Kitano, M., Dudley, M. E., Laurencot, C. M., and Rosenberg, S. A. (2010). Case Report of a Serious Adverse Event Following the Administration of T Cells Transduced With a Chimeric Antigen Receptor Recognizing ERBB2. Mol Ther 18, 843-851.

Morsut, L., Roybal, K. T., Xiong, X., Gordley, R. M., Coyle, S. M., Thomson, M., and Lim, W. A. (2016). Engineering Customized Cell Sensing and Response Behaviors Using Synthetic Notch Receptors. Cell 164, 780-791.

Neelapu, S. S., Locke, F. L., Bartlett, N. L., Lekakis, L. J., Miklos, D. B., Jacobson, C. A., Braunschweig, I., Oluwole, O.O., Siddiqi, T., Lin, Y., et al. (2017). Axicabtagene Ciloleucel CAR T-Cell Therapy in Refractory Large B-Cell Lymphoma. New Engl J Medicine 377, 2531-2544.

Ottoz, D., Rudolf, F., and Stelling, J. (2014). Inducible, tightly regulated and growth condition-independent transcription factor in Saccharomyces cerevisiae. Nucleic Acids Res 42, e130-e130.

Parkhurst, M. R., Yang, J. C., Langan, R. C., Dudley, M. E., Nathan, D.-A. N., Feldman, S. A., Davis, J. L., Morgan, R. A., Merino, M. J., erry, R., et al. (2011). T Cells Targeting Carcinoembryonic Antigen Can Mediate Regression of Metastatic Colorectal Cancer but Induce Severe Transient Colitis. Mol Ther 19, 620-626.

Paul, R., Ewing, C., Robinson, J., Marshall, F., Johnson, K., Wheelock, M., and Isaacs, W. (1997). Cadherin-6, a cell adhesion molecule specifically expressed in the proximal renal tubule and renal cell carcinoma. Cancer Res 57, 2741-2748.

Puri, N., Ahmed, S., Janamanchi, V., Tretiakova, M., Zumba, O., Krausz, T., Jagadeeswaran, R., and Salgia, R. (2007). c-Met Is a Potentially New Therapeutic Target for Treatment of Human Melanoma. Clin Cancer Res 13, 2246-2253.

Qu, X., Liu, J., Zhong, X., Li, X., and Zhang, Q. (2016). Role of AXL expression in non-small cell lung cancer. Oncol Lett 12, 5085-5091.

Robbins, P. F., Li, Y. F., El-Gamil, M., Zhao, Y., Wargo, J. A., Zheng, Z., Xu, H., Morgan, R. A., Feldman, S. A., Johnson, L. A., et al. (2008). Single and Dual Amino Acid Substitutions in TCR CDRs Can Enhance Antigen-Specific T Cell Functions. J Immunol 180, 6116-6131.

Rodgers, D. T., Mazagova, M., Hampton, E. N., Cao, Y., Ramadoss, N. S., Hardy, I. R., Schulman, A., Du, J., Wang, F., Singer, O., et al. (2016). Switch-mediated activation and retargeting of CAR-T cells for B-cell malignancies. Proc National Acad Sci 113, E459-E468.

Roe, K., Gibot, S., and Verma, S. (2014). Triggering receptor expressed on myeloid cells-1 (TREM-1): a new player in antiviral immunity? Front Microbiol 5, 627.

Roovers, R. C., Vosjan, M., Laeremans, T., el Khoulati, R., de Bruin, R., Ferguson, K. M., Verkleij, A. J., van Dongen, G. A., and van en Henegouwen, P. (2011). A biparatopic anti-EGFR nanobody efficiently inhibits solid tumour growth. Int J Cancer 129, 2013-2024.

Rosenberg, S. A., and Restifo, N. P. (2015). Adoptive cell transfer as personalized immunotherapy for human cancer. Science 348, 62-68.

Roybal, K. T., Rupp, L. J., Morsut, L., Walker, W. J., McNally, K. A., Park, J. S., and Lim, W. A. (2016a). Precision Tumor Recognition by T Cells With Combinatorial Antigen-Sensing Circuits. Cell 164,770-779.

Roybal, K. T., Williams, J. Z., Morsut, L., Rupp, L. J., Kolinko, I., Choe, J. H., Walker, W. J., McNally, K. A., and Lim, W. A. (2016b). Engineering T Cells with Customized Therapeutic Response Programs Using Synthetic Notch Receptors. Cell 167, 419-432.e16.

Solly, S., Thomas, -L J, Monge, M., Demerens, C., Lubetzki, C., Gardinier, M., Matthieu, -M J, and Zalc, B. (1996). Myelin/oligodendrocyte glycoprotein (MOG) expression is associated with myelin deposition. Glia 18, 39-48.

Søndergaard, J. N., Nazarian, R., Wang, Q., Guo, D., Hsueh, T., Mok, S., Sazegar, H., MacConaill, L. E., Barretina, J. G., Kehoe, S. M., et al. (2010). Differential sensitivity of melanoma cell lines with BRAFV600E mutation to the specific Raf inhibitor PLX4032. J Transl Med 8, 39.

Srivastava, S., Salter, A. I., Liggitt, D., Yechan-Gunja, S., Sarvothama, M., Cooper, K., Smythe, K. S., Dudakov, J. A., Pierce, R. H., Rader, C., et al. (2019). Logic-Gated ROR1 Chimeric Antigen Receptor Expression Rescues T Cell-Mediated Toxicity to Normal Tissues and Enables Selective Tumor Targeting. Cancer Cell 35, 489-503.e8.

Tanyi, J., Haas, A. R., Beatty, G., Stashwick, C., O'Hara, M. H., Morgan, M., Porter, D. L., Melenhorst, J. J., Plesa, G., Lacey, S. F., et al. (2016). Anti-mesothelin chimeric antigen receptor T cells in patients with epithelial ovarian cancer. Journal of Clinical Oncology 15, 5511.

Tesselaar, K., Xiao, Y., Arens, R., van Schijndel, G. M., Schuurhuis, D. H., Mebius, R. E., Borst, J., and van Lier, R. A. (2003). Expression of the Murine CD27 Ligand CD70 In Vitro and In Vivo. J Immunol 170, 33-40.

Thistlethwaite, F. C., Gilham, D. E., Guest, R. D., Rothwell, D. G., Pillai, M., Burt, D. J., Byatte, A. J., Kirillova, N., Valle, J. W., Sharma, S. K., et al. (2017). The clinical efficacy of first-generation carcinoembryonic antigen (CEACAMS)-specific CAR T cells is limited by poor persistence and transient pre-conditioning-dependent respiratory toxicity. Cancer Immunol Immunother 66, 1425-1436.

Thul, P. J., Åkesson, L., Wiking, M., Mandessian, D., Geladaki, A., Blal, H., Alm, T., Asplund, A., Björk, L., Breckels, L. M., et al. (2017). A subcellular map of the human proteome. Science 356, eaal3321.

Uhlén, M., Fagerberg, L., Hallström, B.M., Lindskog, C., Oksvold, P., Mardinoglu, A., Sivertsson, Å., Kampf, C., Sjöstedt, E., Asplund, A., et al. (2015). Tissue-based map of the human proteome. Science 347, 1260419.

Vogelstein, B., Papadopoulos, N., Velculescu, V. E., Zhou, S., Diaz, L. A., and Kinzler, K. W. (2013). Cancer Genome Landscapes. Science 339, 1546-1558.

Wang, Q. J., Yu, Z., Hanada, K., Patel, K., Kleiner, D., Restifo, N. P., and Yang, J. C. (2017). Preclinical Evaluation of Chimeric Antigen Receptors Targeting CD70-Expressing Cancers. Clin Cancer Res 23, 2267-2276.

Wilkie, S., van Schalkwyk, M. C., Hobbs, S., Davies, D. M., van der Stegen, S. J., Pereira, A. C., Burbridge, S. E., Box, C., Eccles, S. A., and Maher, J. (2012). Dual Targeting of ErbB2 and MUC1 in Breast Cancer Using Chimeric Antigen Receptors Engineered to Provide Complementary Signaling. J Clin Immunol 32, 1059-1070.

Yu, H., Liu, R., Ma, B., Li, X., Yen, H., Zhou, Y., Krasnoperov, V., Xia, Z., Zhang, X., Bove, A., et al. (2015). Axl receptor tyrosine kinase is a potential therapeutic target in renal cell carcinoma. Brit J Cancer 113, 616-625.

Zah, E., Lin, M.-Y., Silva-Benedict, A., Jensen, M. C., and Chen, Y. Y. (2016). T Cells Expressing CD19/CD20 Bispecific Chimeric Antigen Receptors Prevent Antigen Escape by Malignant B Cells. Cancer Immunol Res 4, 498-508.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A host cell comprising:
(a) a first expression cassette comprising:
(i) a first promoter, and
(ii) a first coding sequence encoding a first binding-triggered transcriptional switch (BTTS), wherein the first BTTS comprises a first extracellular binding domain, a transmembrane domain, and a first transcription factor, and wherein binding of the first BTTS to a first antigen on a diseased cell releases the first transcription factor into the cytoplasm of the host cell;
(b) a second expression cassette, wherein transcription of the second expression cassette is induced by the released first transcription factor of (a) and comprises:
(i) a second promoter, wherein the second promoter comprises a binding site for the first transcription factor, and
(ii) a second coding sequence encoding a second BTTS, wherein the second BTTS comprises a second extracellular binding domain, a transmembrane domain, and a second transcription factor, wherein binding of the second BTTS to a second antigen on the diseased cell releases the second transcription factor into the cytoplasm of the host cell; and
(c) a third expression cassette, wherein transcription of the third expression cassette is induced by the released second transcription factor of (b) and comprises:
(i) a third promoter that comprise a binding site for the second transcription factor, and
(ii) a coding sequence encoding a therapeutic protein.

2. The host cell of claim 1, wherein the first transcription factor of (a)(ii) activates transcription of the second promoter more than the second transcription factor of (b)(ii) activates transcription of the third promoter.

3. The host cell of any prior claim, wherein the first and second transcription factors are independently selected from Gal4-, LexA-, Tet-, Lac-, dCas9-, zinc-finger- and TALE-based transcription factors.

4. The system of any prior claim, wherein the first and second BTTSs comprise one or more polypeptides that undergo proteolytic cleavage upon binding to an antigen to release the transcription factor.

5. The cell of any prior claim, wherein the binding-triggered transcriptional switch is a SynNotch receptor, an A2 receptor, a MESA, or another receptor that undergoes binding induced proteolytic cleavage.

6. The host cell of any prior claim, wherein the therapeutic protein of (i) is a protein that, when expressed, is on the surface of the cell.

7. The host cell of claim 6, wherein the therapeutic protein is a protein that, when expressed on the surface of an immune cell, activates the immune cell or inhibits activation of the immune cell when it binds to a third antigen on the diseased cell.

8. The host cell of claim 7, wherein the therapeutic protein is a chimeric antigen receptor (CAR) or a T cell receptor (TCR).

9. The host cell of claim 8, wherein the third expression cassette comprises:
(i) the third promoter, and
(ii) a coding sequence encoding a CAR or TCR, wherein the CAR or TCR comprises a third extracellular binding domain, a transmembrane domain, and an intracellular activation domain, wherein the CAR or TCR activates an immune cell or inhibits activation of the immune cell when it binds to the third antigen on the diseased cell.

10. The host cell of any of claims 1-6, wherein the therapeutic protein is an inhibitory chimeric antigen receptor (iCAR).

11. The host cell of any of claims 1-5, wherein the therapeutic protein is a protein that, when expressed, is secreted by the cell.

12. The host cell of claim 11, wherein the antigen-specific therapeutic protein is an antibody.

13. The host cell of claim 12, wherein the antibody is an immune checkpoint inhibitor.

14. The host cell of claim 11 or 12, wherein the antibody binds to PD1, PD-L1, PD-L2, CTLA4, TIM3 or LAG3.

15. The host cell of claim 11, wherein the therapeutic protein is a secreted peptide.

16. The host cell of claim 15, wherein the secreted peptide is a cytokine.

17. The host cell of claim 11, wherein the secreted peptide is an enzyme.

18. The host cell of claim 17, wherein the enzyme is a superoxide dismutase or protease.

19. The host cell of any of claims 1-5, wherein the therapeutic protein is a protein that, when expressed, is internal to the cell.

20. The host cell of any prior claim, wherein the first and second BTTSs are synNotch receptors that each comprise: (i) an extracellular domain comprising the antigen binding region of an antibody; (ii) a proteolytically cleavable Notch receptor polypeptide comprising one or more proteolytic cleavage sites; and (iii) an intracellular domain comprising the first or second transcription factor, wherein binding of the extracellular domains to the first and second synNotch receptors to the first and second antigens on the diseased cell releases the first and second transcription factors from the first and second BTTSs, respectively.

21. The cell of any prior claim, wherein the cell is an immune cell.

22. The cell of claim 21, wherein the cell is a myeloid or lymphoid cell.

23. The cell of claim 22, wherein the lymphoid cell is a T lymphocyte, a B lymphocyte, a macrophage, a dendritic cell, or a natural killer cell.

24. The cell of any of claims 1-20, wherein the cell is not an immune cell.

25. A method of treating a subject for a disease, the method comprising:
administering to the subject a cell of any of claims 1-24.

26. The method of claim 25, wherein the disease is cancer, an autoimmune disease, fibrosis, a neurodegenerative disease, diabetes, or an infectious disease.
